(19) United States
(12) Patent Application Publication
(10) Pub. No.: US 2024/0085387 A1
(43) Pub. Date: Mar. 14, 2024

(54) MULTI-STAGE TANDEM MASS SPECTROMETRY FOR PROTONATED GLYCAN ISOMER ASSIGNMENT

(71) Applicant: University of Washington, Seattle, WA (US)

(72) Inventors: Abhigya Mookherjee, Seattle, WA (US); Miklos Guttman, Seattle, WA (US)

(73) Assignee: University of Washington, Seattle, WA (US)

(21) Appl. No.: 18/329,495

(22) Filed: Jun. 5, 2023

Related U.S. Application Data
(60) Provisional application No. 63/349,851, filed on Jun. 7, 2022.

(51) Int. Cl. G01N 30/86; H01J 49/00; H01J 49/42

(52) U.S. Cl. CPC G01N 30/8631; G01N 30/8603; G01N 30/8679; H01J 49/0072; H01J 49/424; G01N 2030/027; G01N 2030/862

(57) ABSTRACT

The present disclosure provides a method of analyzing the structure of a glycan sample, the method including: receiving data indicative of one or more spectra of mass-to-charge ratio (m/z) versus relative abundance of the glycan sample from a mass spectrometer (MS) instrument; generating a ratio according to the following Equation:

$$\frac{a}{a + b}$$

wherein a is a magnitude of one or more first peaks in the one or more spectra and b is the magnitude of one or more second peaks in the one or more spectra; determining that the ratio is within a range of a predetermined ratio; based on determining that the ratio is within the range of the predetermined ratio, determining that a predetermined structural characteristic is present in the glycan sample; and outputting an indication of the predetermined structural characteristic in the glycan sample.

### CROSS-REFERENCE TO RELATED APPLICATION

This application claims the benefit of and priority to U.S. Provisional Application Ser. No. 63/349,851, filed on Jun. 7, 2022, which is incorporated by reference herein in its entirety.

### STATEMENT REGARDING FEDERALLY SPONSORED RESEARCH OR DEVELOPMENT

This invention was made with government support under grant Nos. GM127579 and GM 142363, awarded by the National Institutes of Health. The government has certain rights in the invention.

### BACKGROUND OF THE DISCLOSURE

Carbohydrates or “glycans” are abundant biological macro-molecules that make up the majority of the glycocalyx on cells in the form of glycoconjugates. Glycosylation is one of the most common and complex post-translational modifications (PTMs) of secreted and cell surface proteins, enabling diverse biological processes like cell adhesion, cell-cell communication, and immune responses (Varki, A. Biological roles of glycans. Glycobiology 2017, 27, 3-49; Reily, C.; Stewart, T. J.; Renfrow, M. B.; Novak, J. Glycosylation in health and disease. Nat. Rev. Nephrol. 2019, 15, 346-366; Gahmberg, C. G.; Tolvanen, M. Why mammalian cell surface proteins are glycoproteins. Trends Biochem. Sci. 1996, 21, 308-311). Unlike proteins and nucleic acids which are linear polymers built from a template, glycan structures are the sum result of many glycosidase and glycosyltransferase enzymes that can result in polymers with various branching patterns (Varki, A. Evolutionary forces shaping the Golgi glycosylation machinery: why cell surface glycans are universal to living cells. Cold Spring Harbor Perspect. Biol. 2011, 3, a005462). Additional structural complexity stems from glycans having various positional isomers, linkage stereochemistry (α/β anomers), and different compositions of the various monosaccharide building blocks.

Mass spectrometry (MS) is one of the primary techniques used to investigate the immense diversity of glycans, and a great deal of effort has been invested into understanding the gas-phase behavior of carbohydrates. However, MS-based glycan analysis is often confounded by the technique's inability to distinguish among the many potential isomeric structures from a mass measurement.

A thorough characterization of the tandem MS behavior of permethylated oligosaccharides can pave the way for effective discrimination among potential isomers (Sheeley, D. M.; Reinhold, V. N. Structural Characterization of Carbohydrate Sequence, Linkage, and Branching in a Quadrupole Ion Trap Mass Spectrometer: Neutral Oligosaccharides and N-Linked Glycans. Anal. Chem. 1998, 70, 3053-3059). Protonated carbohydrates present additional challenges, as they can undergo rearrangement reactions, most notably migration of fucose during collision-induced dissociation (CID) (Wuhrer, M.; Koeleman, C. A. M.; Hokke, C. H.; Deelder, A. M. Mass spectrometry of proton adducts of fucosylated N-glycans: fucose transfer between antennae gives rise to misleading fragments. Rapid Commun. Mass Spectrom. 2006, 20, 1747-1754). This migration process is still under investigation (Mucha, E.; Lettow, M.; Marianski, M.; Thomas, D. A.; Struwe, W. B.; Harvey, D. J.; Meijer, G.; Seeberger, P. H.; von Heiden, G.; Pagel, K. Fucose Migration in Intact Protonated Glycan Ions: A Universal Phenomenon in Mass Spectrometry. Angew. Chem., Int. Ed. 2018, 57, 7440-7443; Sastre Toraño, J.; Gagarinov, I. A.; Vos, G. M.; Broszeit, F.; Srivastava, A. D.; Palmer, M.; Langridge, J. I.; Aizpurua-Olaizola, O.; Somovilla, V. J.; Boons, G.-J. Ion-Mobility Spectrometry Can Assign Exact Fucosyl Positions in Glycans and Prevent Misinterpretation of Mass-Spectrometry Data After Gas-Phase Rearrangement. Angew. Chem., Int. Ed. 2019, 58, 17616-17620; Lettow, M.; Mucha, E.; Manz, C.; Thomas, D. A.; Marianski, M.; Meijer, G.; von Heiden, G.; Pagel, K. The role of the mobile proton in fucose migration. Anal. Bioanal. Chem. 2019, 411, 4637-4645), and a general structural understanding of the even simple protonated carbohydrate systems is only now starting to emerge (Bythell, B. J.; Abutokaikah, M. T.; Wagoner, A. R.; Guan, S.; Rabus, J. M. Cationized Carbohydrate Gas-Phase Fragmentation Chemistry. J. Am. Soc. Mass Spectrom. 2017, 28, 688-70).

For intact glycoconjugates (e.g., glycopeptides), which are typically analyzed as protonated ions, tandem MS is incapable of providing full information on the branching pattern, linkage information, or stereochemistry of the glycan (Pap, A.; Klement, E.; Hunyadi-Gulyas, E.; Darula, Z.; Medzihradszky, K. F. Status Report on the High-Throughput Characterization of Complex Intact O-Glycopeptide Mixtures. J. Am. Soc. Mass Spectrom. 2018, 29, 1210-1220). Understanding glycobiology at the highest level of detail, including site-specific localization of all glycan structural isomers within glycoproteins (Schumacher, K. N.; Dodds, E. D. A case for protein-level and site-level specificity in glycoproteomic studies of disease. Glycoconjugate J. 2016, 33, 377-385), can be achieved by a thorough understanding of the properties of carbohydrate fragment ions. Tools complementary to MS including ion mobility (IM) and spectroscopic techniques like cryogenic ion spectroscopy and ultraviolet photodissociation have emerged as powerful gas-phase techniques with potential applications in glycobiology (Clowers, B. H.; Dwivedi, P.; Steiner, W. E.; Hill, H. H.; Bendiak, B. Separation of Sodiated Isobaric Disaccharides and Trisaccharides Using Electrospray Ionization-Atmospheric Pressure Ion Mobility-Time of Flight Mass Spectrometry. J. Am. Soc. Mass Spectrom. 2005, 16, 660-669; Dwivedi, P.; Bendiak, B.; Clowers, B. H.; Hill, H. H. Rapid Resolution of Carbohydrate Isomers by Electrospray Ionization Ambient Pressure Ion Mobility Spectrometry-Time-of-Flight Mass Spectrometry (ESI-APIMS-TOFMS). J. Am. Soc. Mass Spectrom. 2007, 18, 1163-1175; Brodbelt, J. S. Photodissociation mass spectrometry: new tools for characterization of biological molecules. Chem. Soc. Rev. 2014, 43, 2757-2783; Tan, Y.; Polfer, N. C. Linkage and Anomeric Differentiation in Trisaccharides by Sequential Fragmentation and Variable-Wavelength Infrared Photodissociation. J. Am. Soc. Mass Spectrom. 2015, 26, 359-368; Gray, C. J.; Schindler, B.; Migas, L. G.; Picm̆ anova\acute{,} M.; Allouche, A. R.; Green, A. P.; Mandal, S.; Motawia, M. S.; Sańchez-Peŕez, R.; Bjarnholt, N.; Møller, B. L.; Rijs, A. M.; Barran, P. E.; Compagnon, I.; Eyers, C. E.; Flitsch, S. L. Bottom-Up Elucidation of Glycosidic Bond Stereochemistry. Anal. Chem. 2017, 89, 4540-4549; Mucha, E.; Gonzaĺez Floŕez, A. I.; Marianski, M.; Thomas, D. A.; Hoffmann, W.; Struwe, W. B.; Hahm, H. S.; Gewinner, S.; Schöllkopf, W.; Seeberger, P. H.; von Heiden, G.; Pagel, K. Glycan Fingerprinting via Cold-Ion Infrared Spectroscopy. Angew. Chem., Int. Ed. 2017, 56, 11248-11251; Riggs, D. L.; Hofmann, J.; Hahm, H. S.; Seeberger, P. H.; Pagel, K.; Julian, R. R. Glycan Isomer Identification Using Ultraviolet Photodissociation Initiated Radical Chemistry. Anal. Chem. 2018, 90, 11581-11588; Ben Faleh, A.; Warnke, S.; Rizzo, T. R. Combining Ultrahigh-Resolution Ion-Mobility Spectrometry with Cryogenic Infrared Spectroscopy for the Analysis of Glycan Mixtures. Anal. Chem. 2019, 91, 4876-4882; Gray, C. J.; Migas, L. G.; Barran, P. E.; Pagel, K.; Seeberger, P. H.; Eyers, C. E.; Boons, G.-J.; Pohl, N. L. B.; Compagnon, I.; Widmalm, G.; Flitsch, S. L. Advancing Solutions to the Carbohydrate Sequencing Challenge. J. Am. Chem. Soc. 2019, 141, 14463-14479).

Ion mobility with mass spectrometry (IM-MS) separates ions by their charge, size, and shape due to interactions with an inert gas. IM-MS has shed light on the structural details of carbohydrates in the gas phase (Gray, C. J et al., 2017; Mookherjee, A.; Uppal, S. S.; Guttman, M. Dissection of Fragmentation Pathways in Protonated N-Acetylhexosamines. Anal. Chem. 2018, 90, 11883-11891), and the emerging next-generation of high-resolution IM-MS shows much promise at resolving even subtle isomeric variants within larger glycans (Ben Faleh, A et al., 2019; Ujma, J.; Ropartz, D.; Giles, K.; Richardson, K.; Langridge, D.; Wildgoose, J.; Green, M.; Pringle, S. Cyclic Ion Mobility Mass Spectrometry Distinguishes Anomers and Open-Ring Forms of Pentasaccharides. J. Am. Soc. Mass Spectrom. 2019, 30, 1028-1037; Wojcik, R.; Webb, I. K.; Deng, L.; Garimella, S. V.; Prost, S. A.; Ibrahim, Y. M.; Baker, E. S.; Smith, R. D. Lipid and Glycolipid Isomer Analyses Using Ultra-High Resolution Ion Mobility Spectrometry Separations. Int. J. Mol. Sci. 2017, 18, 183). Gas-phase spectroscopy is emerging to define structural variations in carbohydrates and their structures on its own or in combination with IM-MS (Mucha, E et al., 2017; Ben Faleh, A et al., 2019; Scutelnic, V.; Rizzo, T. R. Cryogenic Ion Spectroscopy for Identification of Monosaccharide Anomers. J. Phys. Chem. A 2019, 123, 2815-2819; Khanal, N.; Masellis, C.; Kamrath, M. Z.; Clemmer, D. E.; Rizzo, T. R. Cryogenic IR spectroscopy combined with ion mobility spectrometry for the analysis of human milk oligosaccharides. Analyst 2018, 143, 1846-1852; Warnke, S.; Ben Faleh, A.; Pellegrinelli, R. P.; Yalovenko, N.; Rizzo, T. R. Combining ultra-high resolution ion mobility spectrometry with cryogenic IR spectroscopy for the study of biomolecular ions. Faraday Discuss. 2019, 217, 114-125).

Gas-phase hydrogen/deuterium exchange (gHDX), which tracks the exchangeability of various labile protons in an ion, has also been shown effective at distinguishing isomers including carbohydrates (Ausloos, P.; Lias, S. G. Thermoneutral isotope-exchange reactions of cations in the gas phase. J. Am. Chem. Soc. 1981, 103, 3641-3647; Cheng, X.; Fenselau, C. Hydrogen/deuterium exchange of mass-selected peptide ions with ND3 in a tandem sector mass spectrometer. Int. J. Mass Spectrom. Ion Processes 1992, 122, 109-119; Gard, E.; Green, M. K.; Bregar, J.; Lebrilla, C. B. Gas-phase hydrogen/deuterium exchange as a molecular probe for the interaction of methanol and protonated peptides. J. Am. Soc. Mass Spectrom. 1994, 5, 623-631; Zhang, J.; Brodbelt, J. S. Gas-Phase Hydrogen/Deuterium Exchange and Conformations of Deprotonated Flavonoids and Gas-Phase Acidities of Flavonoids. J. Am. Chem. Soc. 2004, 126, 5906-5919; Uppal, S. S.; Beasley, S. E.; Scian, M.; Guttman, M. Gas-Phase Hydrogen/Deuterium Exchange for Distinguishing Isomeric Carbo-hydrate Ions. Anal. Chem. 2017, 89, 4737-4742; Majuta, S. N.; Li, C.; Jayasundara, K.; Kiani Karanji, A.; Attanayake, K.; Ranganathan, N.; Li, P.; Valentine, S. J. Rapid Solution-Phase Hydrogen/Deuterium Exchange for Metabolite Compound Identification. J. Am. Soc. Mass Spectrom. 2019, 30, 1102-1114; Liyanage, O. T.; Quintero, A. V.; Hatvany, J. B.; Gallagher, E. S. Distinguishing Carbohydrate Isomers with Rapid Hydrogen/Deuterium Exchange-Mass Spectrometry. J. Am. Soc. Mass Spectrom. 2020, DOI: 10.1021/jasms.0c00314).

Thanks to their ability to resolve structural isomers, these new techniques have also shown examples of multiple distinct gas-phase chemical structures obtained for a single distinct carbohydrate, often attributed to the site of protonation or deprotonation (Struwe, W. B.; Baldauf, C.; Hofmann, J.; Rudd, P. M.; Pagel, K. Ion mobility separation of deprotonated oligosaccharide isomers—evidence for gas-phase charge migration. Chem. Commun. 2016, 52, 12353-12356). Recent studies have further illustrated the complexity of fragmentation of protonated carbohydrates. Gray et al. using IM-MS found that B-ion fragments of carbohydrates retain information on the stereochemistry at their reducing end even after losing their reducing end (“anomeric memory”) (Gray, C. J. et al., 2017). This is thought to arise from the generation of different isomeric fragment ion structures, whose ensemble is affected by the structure of the precursor. Similar “memory effects” were also observed by both IM-MS and MSⁿ relative fragmentation propensities even after multistage MS (Mookherjee, A. et al., 2018).

### BRIEF DESCRIPTION OF THE SEVERAL VIEWS OF THE DRAWINGS

Many of the drawings submitted herein are better understood in color. Applicant considers the color versions of the drawings as part of the original submission and reserves the right to present color images of the drawings in later proceedings.

FIG. 1 illustrates an example environment for analyzing the structure of a glycan sample.

FIG. 2 illustrates an example of a device.

FIG. 3 illustrates via a flow chart the actions that are performed in the presently disclosed method.

FIG. 4 is a schematic showing the inherent structural heterogeneity of glycans, showing structural diversity in terms of composition, the connectivity of O-glycosidic linkages and the configuration of anomeric carbons (α vs β) in an oligosaccharide of the glycan.

FIG. 5 illustrates an estimation of the error associated with ratios from the total ion counts.

FIG. 6 shows how the ratios of 0.37, 0.81, or 0.73 for (m/z 138)/(m/z 138+m/z 144) point to different structural characteristics of the oligosaccharide portion of a glycan.

FIG. 7 shows a plot of ratio of MS³ ratio of (m/z 84+m/z 126)/(sum of all m/z 204 fragments) vs collision energy.

FIGS. 8A-I Illustrate examples of MSn spectra of various Gal-GlcNAc and Fuc-GlcNAc disaccharides.

FIG. 8A shows the MSn spectra of Gal(β1-4)GlcNAc (top), Gal(β1-6)GlcNAc (middle), and Gal(β1-3)GlcNAc (bottom) from the MSn−1 peak of m/z 384.

FIG. 8B shows the MSn spectra of Fuc(α1-4)GlcNAc (top), Fuc(α1-6)GlcNAc (middle), and Fuc(α1-3)GlcNAc (bottom) from the MSn−1 peak of m/z 368.

FIG. 8C shows the MSn spectra of Gal(β1-4)GlcNAc (top), Gal(β1-6)GlcNAc (middle), and Gal(β1-3)GlcNAc (bottom) from the MSn−1 peak of m/z 38443664.

FIG. 8D shows the MSn spectra of Fuc(α1-4)GlcNAc (top), Fuc(α1-6)GlcNAc (middle), and Fuc(α1-3)GlcNAc (bottom) from the MSn−1 peak of 36843504.

FIG. 8E shows the MSn spectra of Gal(β1-4)GlcNAc (top), Gal(β1-6)GlcNAc (middle), and Gal(β1-3)GlcNAc (bottom) from the MSn−1 peak of 384436642044.

FIG. 8F shows the MSn spectra of Fuc(α1-4)GlcNAc (top), Fuc(α1-6)GlcNAc (middle), and Fuc(α1-3)GlcNAc (bottom) from the MSn−1 peak of 368→350→204→.

FIG. 8G shows the anomers of the disaccharides Gal(β1-4)GlcNAc (top), Gal(β1-6)GlcNAc (middle), and Gal(β1-3)GlcNAc (bottom) from the MSn spectra derived from MSn−1 peak of 384→366→204→ were resolved by HILIC and analyzed online by MS4.

FIG. 8H shows the anomers of the disaccharides Fuc(α1-4)GlcNAc (top), Fuc(α1-6)GlcNAc (middle), and Fuc(α1-3)GlcNAc (bottom) from the MSn spectra derived from MSn−1 peak of 368→350→204→ were resolved by HILIC and analyzed online by MS4.

FIG. 8I shows a legend for FIGS. 8A-H. The symbols show the three monosaccharides GlcNAc (in blue), galactose (in yellow), and fucose (in red) that form the six disaccharides, while the solid/dashed lines denote the glycosidic bond between the monosaccharides with the angle of the line specifying a linkage configuration. Solid lines denote a beta (β) linkage while the dashed lines denote the alpha (α) linkage along with the originating carbon atom and ending carbon atom numbered involved in the linkage.

FIGS. 9A-H illustrate arrival time distributions (ATDs) in N₂ of various m/z fractions and the IMMSn analysis of LC resolved anomers of Gal(β1-x)GlcNAc and Fuc(α1-x)GlcNAc.

FIG. 9A shows ATD in N₂ of m/z 366 for Gal(β1-x)GlcNAc.

FIG. 9B shows ATD in N₂ of m/z 350 for Fuc(α1-x)GlcNAc.

FIG. 9C shows ATD in N₂ of m/z 204 from 366 for Gal(β1-x)GlcNAc.

FIG. 9D shows ATD in N₂ of m/z 204 from 350 for Fuc(α1-x)GlcNAc.

FIG. 9E shows the IMMSn analysis of LC resolved anomers of Gal-GlcNAc at ATD of m/z 366.

FIG. 9F shows the IMMSn analysis of LC resolved anomers of Gal(β1-x)GlcNAc at ATD of m/z 204.

FIG. 9G shows the IMMSn analysis of LC resolved anomers of Fuc-GlcNAc at ATD of m/z 350.

FIG. 9H shows the IMMSn analysis of LC resolved anomers of Fuc-GlcNAc at ATD of m/z 204.

FIGS. 10A-F Illustrate examples gHDX analysis of various m/z fractions for Gal(β1-x)GlcNAc and Fuc(α1-x)GlcNAc disaccharides and examples of mass gHDX spectra as a function of collision energy (CE).

FIG. 10A shows gHDX analysis of m/z 366 of Gal(β1-x)GlcNAc disaccharide.

FIG. 10B shows gHDX analysis of m/z 350 of Fuc(α1-x)GlcNAc disaccharide.

FIG. 10C shows gHDX analysis of m/z 204 of Gal(β1-x)GlcNAc disaccharide.

FIG. 10D shows gHDX analysis of m/z 204 of Fuc(α1-x)GlcNAc disaccharide.

FIG. 10E shows an example mass gHDX spectrum from the m/z 366 peak of Gal(β1-6)GlcNAc generated from the precursor ion (m/z 386) using a CE of 5.

FIG. 10F shows an example mass gHDX spectrum from the m/z 366 peak of Gal(β1-6)GlcNAc generated from the precursor ion (m/z 386) using a CE of 10.

FIG. 11 illustrates a proposed structural rationalization of linkage memory. GlcNAc linked to another monosaccharide at either the 6 (magenta), 4 (blue), or 3 (green) position undergoes a loss of the reducing end hydroxyl upon fragmentation.

FIGS. 12A-C illustrate structures of linkage isomers and fragmentation spectrum of ¹⁸O labeled Fuc-GlcNAc.

FIG. 12A illustrates the chemical structures of the 3 linkage isomers of Gal-GlcNAc.

FIG. 12B illustrates the structure of Fuc(α1-6)GlcNAc showing the primary fragmentations in dashed lines.

FIG. 12C illustrates the CID fragmentation spectrum of Fuc(α1-6)GlcNAc with or without ¹⁸O labeling at the reducing end oxygen (m/z 370 top panel, and 368 bottom panel). Red labels are used for ions containing the ¹⁸O label.

FIG. 13 illustrates separation of α/β anomers of Gal-GlcNAc and Fuc-GlcNAc by HILIC.

FIG. 14 illustrates LC-MS3 of the α/β anomers of GalGlcNAc m/z 366 (top panel) and FucGlcNAc m/z 350 (bottom panel) ions.

FIGS. 15A-B illustrate collision energy dependence for the ATDs of Gal(β1-6)GlcNAc m/z 366 and Fuc(α1-6)GlcNAc m/z 350.

FIG. 15A illustrates the ATDs of the Gal(β1-6)GlcNAc m/z 366 at various collision energies from 16 to (top to bottom traces).

FIG. 15B illustrates the ATDs of the Fuc(α1-6)GlcNAc m/z 350 at various collision energies from 16 to (top to bottom traces).

FIGS. 16A-B illustrate deconvolution of the multiple arrival time distributions of Gal(β1-6)GlcNAc m/z 366, and Fuc(α1-6)GlcNAc m/z 350.

FIG. 16A illustrates ATD of Gal(β1-6)GlcNAc m/z 366 in N₂, He, and CO₂.

FIG. 16B illustrates ATD of Fuc(α1-6)GlcNAc m/z 350, in N₂, He, and CO₂.

FIG. 17 illustrates ATDs of the m/z 138 ion from each isomer of GalGlcNAc.

FIGS. 18A-B illustrate gHDX spectra of Gal(β1-x)GlcNAc m/z 366 and 204.

FIG. 18A illustrates gHDX spectra of m/z 366 for each of the linkages examined of Gal-GlcNAc.

FIG. 18B illustrates gHDX spectra of m/z 204 for each of the linkages examined of Gal-GlcNAc.

FIGS. 19A-B illustrate gHDX spectra of Fuc(α1-x)GlcNAc m/z 350 and 204.

FIG. 19A illustrates gHDX spectra of m/z 350 for each of the three linkages examined of Fuc-GlcNAc.

FIG. 19B illustrates gHDX spectra of m/z 204 for each of the three linkages examined of Fuc-GlcNAc.

FIGS. 20A-E illustrate collision energy effects on the gHDX kinetics of the m/z 366, 350 and 204 fragments.

FIG. 20A illustrates a deuterium uptake plot of m/z 366 of Gal-GlcNAc.

FIG. 20B illustrates a deuterium uptake plot of m/z 350 of Fuc-GlcNAc.

FIG. 20C illustrates a deuterium uptake plot of subsequent m/z 204 ions from GalGlcNAc.

FIG. 20D illustrates a deuterium uptake plot of subsequent m/z 204 ions from FucGlcNAc.

FIG. 20E illustrates an overlay of the deuterium uptake for the m/z 204 ion from the six different disaccharides at the high CE energies.

FIG. 21 illustrates NMR spectra of Fuc-GlcNAc and Gal-GlcNAc disaccharides. The peak from the residual formic acid is depicted with a red asterisk.

FIG. 22 shows an overview of an example of the LC-MSn approach applied to an actual glycopeptide from a glycoprotein digest (indicated in the top inset with glycosylated serine in purple).

FIG. 23 illustrates the formation of ethyl glucoside by the combination of glucose and ethanol (with water as a byproduct).

FIG. 24 illustrates the overview MSⁿ for obtaining maximum structural information on glycopeptides.

FIG. 25 illustrates example spectra of an O-linked glycopeptide after fragmentation.

FIG. 26 illustrates that Protein Metrics' Byos platform that offers about 25 predefined workflows, which prompt the users for required inputs and parameter settings, and then launch the software modules to run the data on the desktop or in the Cloud.

FIG. 27 illustrates that Byonic annotates glycopeptides MS/MS spectra including diagnostic carbohydrate peaks.

FIGS. 28A-C illustrate the benefits of ion trap wherein MSⁿ is used for monosaccharide differentiation.

FIG. 28A illustrates ion trap CID MS/MS spectra of the m/z 204 ion from GlcNAc and GalNAc.

FIG. 28B illustrates the intensity ratio of m/z 138 compared to the sum of 138 and 144 collected at different collision energies (CE).

FIG. 28C illustrates the intensity ratio of m/z 84 and 126 compared to the sum of all fragments of m/z 204 collected at different collision energy (CE). Data for GlcNac and GalNAc are shown collected using different types of collision cells (Q-TOF, HCD, and CID on both a Thermo Fusion and LTQ instrument).

FIGS. 29A-F illustrate ion trap CID fragment propensity examples from various di- and trisaccharides.

FIG. 29A illustrates MS³ fragment spectra of the m/z 657 ion from three isomers of Neu5Ac-Gal-GlcNAc.

FIG. 29B illustrates the ratio of the intensity of m/z 204: (204+274+292+454) that is plotted at different CE.

FIG. 29C illustrates MS³ fragment spectra of the m/z 366 ion from the isomers of Gal-GlcNAc.

FIG. 29D illustrates that the ratio of the intensity of m/z (138+168): (126+138+168+186) reveals a characteristic ratio for each linkage isomer that is not significantly influenced by CE.

FIG. 29E illustrates MS⁴ fragment spectra of m/z 204 that is isolated from the three linkage isomers in FIG. 29C.

FIG. 29F illustrates that the intensity ratios of the m/z (84+126): (all 204 fragment ions) is characteristically different from the (α1-6) linkage isomer.

FIG. 30 provides an overview of the ions of interest and information content obtained from each stage of MS.

### DETAILED DESCRIPTION

As used herein, “glycan” (or “polysaccharide”) may refer to synonyms meaning “compounds consisting of a large number of monosaccharides linked glycosidically. Furthermore, in practice “glycan” can refer to the carbohydrate portion of a glycoconjugate, such as a glycoprotein, glycolipid, or a proteoglycan, even if the carbohydrate is only an oligosaccharide. Glycans can consist solely of O-glycosidic linkages of monosaccharides. Glycans can be homo-or heteropolymers of monosaccharide residues, and can be linear or branched.

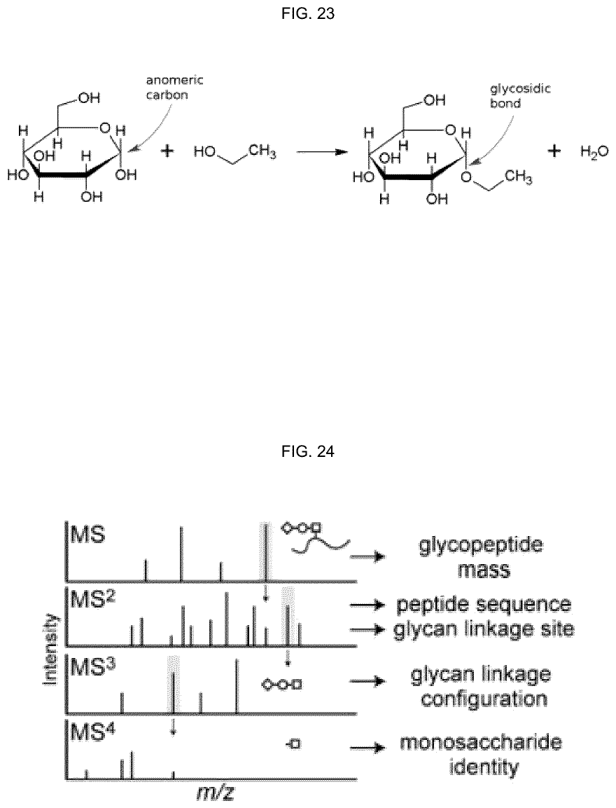

As used herein, an “O-glycosidic bond” or “O-glycosidic linkage” (or “glycosidic bond” or “glycosidic linkage”) may refer to a type of ether bond that joins a carbohydrate (sugar) molecule to another group, which may or may not be another carbohydrate. A glycosidic bond is formed between the hemiacetal or hemiketal group of a saccharide (or a molecule derived from a saccharide) and the hydroxyl group of some compound such as an alcohol. A substance containing a glycosidic bond is a glycoside. For example, the formation of ethyl glucoside by the combination of glucose and ethanol (with water as a byproduct) is shown in FIG. 23. The reaction often favors formation of the α-glycosidic bond as shown due to the anomeric effect.

As used herein, “anomer” or “anomers” may refer to a pair of near-identical stereoisomers that differ at only the anomeric carbon, the carbon that bears the aldehyde or ketone functional group in the sugar's open-chain form. “Anomerization” is the process of conversion of one anomer to the other. In various examples, different anomers have different physical properties, melting points and specific rotations.

As used herein, “isomer” or “isomers” may refer to molecules or polyatomic ions with identical molecular formulae—that is, same number of atoms of each element—but distinct arrangements of atoms in space. “Isomerism” is existence or possibility of isomers.

As used herein, “sialylation” may refer to the covalent addition of sialic acid to the terminal end of glycoproteins. “Sialylated glycan” refers to a glycan that has undergone sialylation.

As used herein, “sialic acid” or “sialic acids” may refer to a class of alpha-keto acid sugars with a nine-carbon backbone. The sialic acid family includes derivatives of the nine-carbon sugar neuraminic acid (Neu), having the structure:

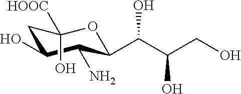

The N- or O-substituted derivatives of Neu form many examples of sialic acids. Examples include N-Acetylneuraminic acid (Neu5Ac; or NeuAc) and 2-Keto-3-deoxyunonic acid (Kdn) having the structures:

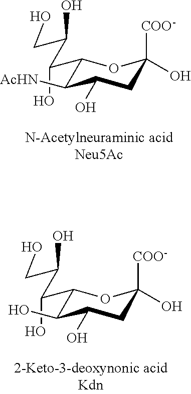

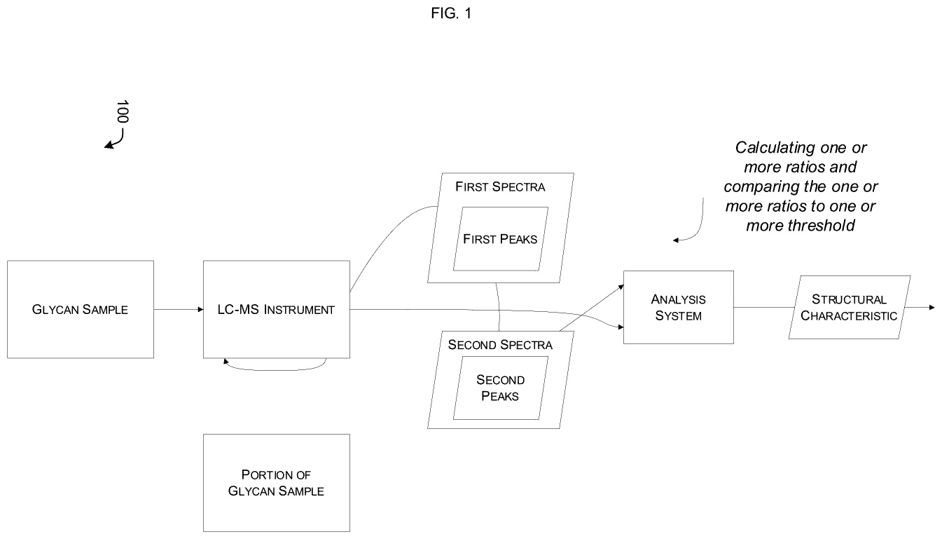

FIG. 1 illustrates an example environment 100 for analyzing the structure of a glycan sample. As shown in FIG. 1, in various implementations, an MS or an LC-MS instrument receives a carbohydrate sample. The carbohydrate sample, for instance, is a glycan sample. The LC-MS instrument may include an LC component and an MS component. In the case of an LC-MS instrument, the LC component is coupled to the MS component. The MS component is configured to generate mass-to-charge ratio (m/z) versus relative abundance spectra based on the sample that as provided as input to the MS instrument.

In particular cases, the LC-MS instrument generates a first spectrum that includes first peaks. The first spectrum may be represented as data. In various implementations, the first peaks correspond to specific abundance values in the first spectrum that correspond to predetermined m/z values. The predetermined m/z values correspond to specific known structural characteristics.

In various implementations, the MS and/or the LC-MS instrument is configured to perform multiple MS rounds on the carbohydrate sample. For instance, the MS, or LC-MS instrument performs an MS1 round to generate the first spectrum. Further, the MS, or LC-MS instrument performs an MS2 round on at least a portion of the carbohydrate sample to generate a second spectrum including second peaks. The portion of the carbohydrate sample utilized by the MS or LC-MS instrument in the MS2 round, for instance, is defined according to one or more of the first peaks in the first spectrum. Although FIG. 1 illustrates the results of an MS1 round and an MS2 round, in various implementations, the MS or LC-MS instrument performs MSn rounds on the carbohydrate sample, to obtain n spectra, wherein n is a positive integer that is greater than 1. In some cases, n is four or less.

The first spectrum and the second spectrum are input into an analysis system for further processing. In various implementations, the analysis system is embodied in at least one computing device. For instance, one or more processors are configured to execute the functions of the analysis system. In some cases, the processor(s) execute instructions stored in memory, a non-transitory computer readable medium, or both. Although the analysis system is illustrated, in FIG. 1, as being separate from the MS or LC-MS instrument, implementations are not so limited. For example, in some implementations, a single apparatus includes the MS or LC-MS instrument and the analysis system.

The analysis system is configured to identify a structural characteristic of the carbohydrate sample by analyzing the first spectra and the second spectra. In various implementations, the analysis system identifies relative abundance values represented by at least one of the first peaks and/or at least one of the second peaks. In particular cases, the analysis system identifies the relative abundance values in the first peaks and/or second peaks that correspond to two or more predetermined m/z values. Further, the analysis system may calculate ratios of two or more of the relative abundance values. In some cases, the ratios are between a first relative abundance value and a sum of two or more second relative abundance values among the relative abundance values. Thus, the analysis system generates one or more ratios corresponding to the original carbohydrate sample.

In various implementations of the present disclosure, the analysis system identifies the structural characteristic based on the one or more ratios. For example, the analysis system may determine that an example ratio of the carbohydrate sample is within a predetermined range of a predetermined ratio corresponding to a predetermined structural characteristic. In some cases, the analysis system compares multiple ratios of the carbohydrate sample to multiple predetermined ratios in order to determine whether the carbohydrate sample has one or more of multiple predetermined characteristics. Specific examples of the predetermined m/z values, the predetermined ratios, the predetermined ranges, and the predetermined characteristics are described below. In some cases, the predetermined range associated with a predetermined ratio is adjusted based on the variability or uncertainty in the measurement of the ratio. For instance, the predetermined range may be adjusted based the total ion counts of a given MSⁿ spectrum performed by the LC-MS instrument.

In various implementations, the analysis system outputs the structural characteristic, the first spectra, the second spectra, or any combination thereof, to a user. For example, the analysis system may include a display that visually presents information to the user.

According to some cases, the analysis system outputs the structural characteristic, the first spectra, the second spectra, or any combination thereof, to an external device, such as an external computing device. For instance, the analysis system includes one or more transceivers that encode data representing information into one or more communication signals that the transceiver(s) transmit to the external device. In some examples, the external device performs additional processing on the data, or outputs the data to a user.

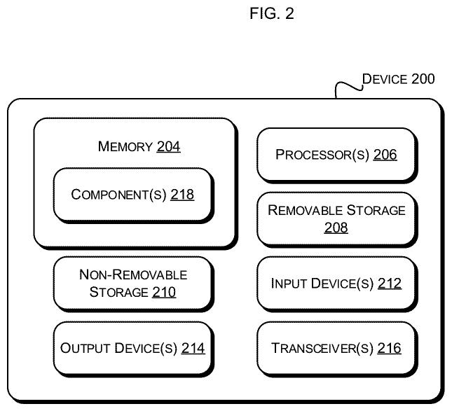

FIG. 2 illustrates an example of a device 200. The device 200, for instance, may be part of, or otherwise embody, the MS or LC-MS instrument and/or the analysis system described above with reference to FIG. 1. The device 200 includes any of memory 204, processor(s) 206, removable storage 208, non-removable storage 210, input device(s) 212, output device(s) 214, and transceiver(s) 216. The device 200 may be configured to perform various methods and functions disclosed herein.

The memory 204 may include component(s) 218. The component(s) 218 may include at least one of instruction(s), program(s), database(s), software, operating system(s), etc. In some implementations, the component(s) 218 include instructions that are executed by processor(s) 206 and/or other components of the device 200. For example, the component(s) 218 may include instructions for generating spectra, generating ratios based on predetermined m/z values, comparing ratios to one or more predetermined ratios, and identifying structural characteristics.

In some embodiments, the processor(s) 206 include a central processing unit (CPU), a graphics processing unit (GPU), or both CPU and GPU, or other processing unit or component known in the art.

The device 200 may also include additional data storage devices (removable and/or non-removable) such as, for example, magnetic disks, optical disks, or tape. Such additional storage is illustrated in FIG. 2 by removable storage 208 and non-removable storage 210. Tangible computer-readable media can include volatile and non-volatile, removable and non-removable media implemented in any method or technology for storage of information, such as computer readable instructions, data structures, program modules, or other data. The memory 204, the removable storage 208, and the non-removable storage 210 are all examples of computer-readable storage media. Computer-readable storage media include, but are not limited to, Random Access Memory (RAM), Read-Only Memory (ROM), Electrically Erasable Programmable Read-Only Memory (EEPROM), flash memory, or other memory technology, Compact Disk Read-Only Memory (CD-ROM), Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device 200. Any such tangible computer-readable media can be part of the device 200.

The device 200 may be configured to communicate over a telecommunications network using any common wireless and/or wired network access technology. Moreover, the device 200 may be configured to run any compatible device Operating System (OS), including but not limited to, Microsoft Windows Mobile, Google Android, Apple iOS, Linux Mobile, as well as any other common mobile device OS.

The device 200 also can include input device(s) 212, such as an LC component, an MS component, an LC-MS instrument, one or more analog-to-digital converters (ADCs), keypad, a cursor control, a touch-sensitive display, voice input device, etc. The device 200 further includes output device(s) 214 such as a display, speakers, printers, etc.

As illustrated in FIG. 2, the device 200 also includes one or more wired or wireless transceiver(s) 216. For example, the transceiver(s) 216 can include a network interface card (NIC), a network adapter, a Local Area Network (LAN) adapter, or a physical, virtual, or logical address to connect to various network components, for example. To increase throughput when exchanging wireless data, the transceiver(s) 216 can utilize multiple-input/multiple-output (MIMO) technology. The transceiver(s) 216 can include any sort of wireless transceivers capable of engaging in wireless, radio frequency (RF) communication. The transceiver(s) 216 can also include other wireless modems, such as a modem for engaging in Wi-Fi, WiMAX, Bluetooth, infrared communication, and the like. The transceiver(s) 216 may include transmitter(s), receiver(s), or both.

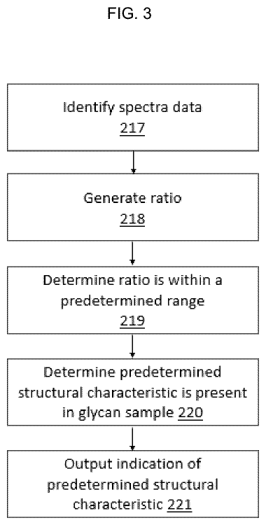

FIG. 3 illustrates via a flow chart example actions for analyzing a carbohydrate sample. For instance, the actions illustrated in FIG. 3 could be performed by an entity including the LC-MS instrument, the LC component, the MS component, the analysis system, a computing device, at least one processor, or any combination thereof.

At 217, the entity identifies spectra data. In various implementations, the spectra data is indicative of one or more spectra of m/z versus relative abundance of a glycan sample. The spectra, for instance, are generated by an MS instrument, such as an LC-MS instrument. In some cases, the instrument includes an ion trap MS instrument, a collision-induced dissociation (CID) MS instrument, or the like. The glycan sample, in various cases, can include a protonated glycan including an oligosaccharide.

According to various implementations, the spectra are generated using multiple MS rounds. For example, n spectra are generated based on respective MSn rounds, wherein n is an integer that is greater than 1 or 2. In some cases, n is in a range of 2 to 4.

At 218, the entity generates a ratio. For instance, the entity calculates the ratio using the following equation:

$$\frac{a}{a + b} \qquad ( 1 )$$

Wherein a is a magnitude of one or more first peaks in the spectra and b is the magnitude of one or more second peaks in the spectra. The magnitudes of the peaks, for instance, are magnitudes of relative abundance at predetermined m/z ratios.

At 219, the entity determines that the ratio is within a predetermined range. The predetermined range, in some implementations, is determined based on a number of ion counts identified by the instrument generating the spectra. For instance, the predetermined range is dependent on the amount of the glycan sample analyzed by the instrument.

At 220, the entity determines that a predetermined structural characteristic is present in the glycan sample. For instance, the predetermined structural characteristic is an identity of the glycan, a connectivity of linkages within a polysaccharide of the glycan, a configuration of one or more monosaccharides in the glycan, or any combination thereof.

At 221, the entity outputs an indication of the predetermined structural characteristic. In some implementations, the entity visually presents the indication on a display. In some cases, the entity outputs the indication by a speaker as an audio signal. According to various cases, the entity transmits a communication signal encoding the indication to an external device.

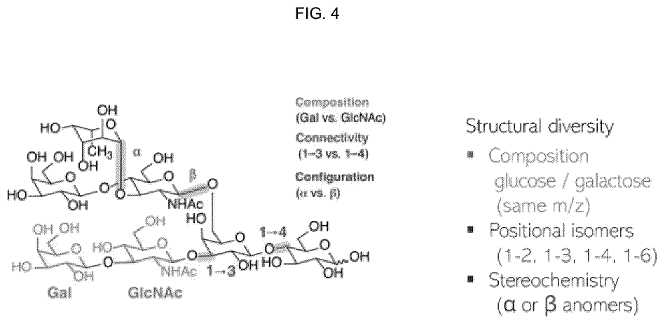

FIG. 4 is a schematic showing the inherent structural heterogeneity of glycans, showing structural diversity in terms of composition, the connectivity of O-glycosidic linkages and the configuration of anomeric carbons (α vs β) in an oligosaccharide of the glycan.

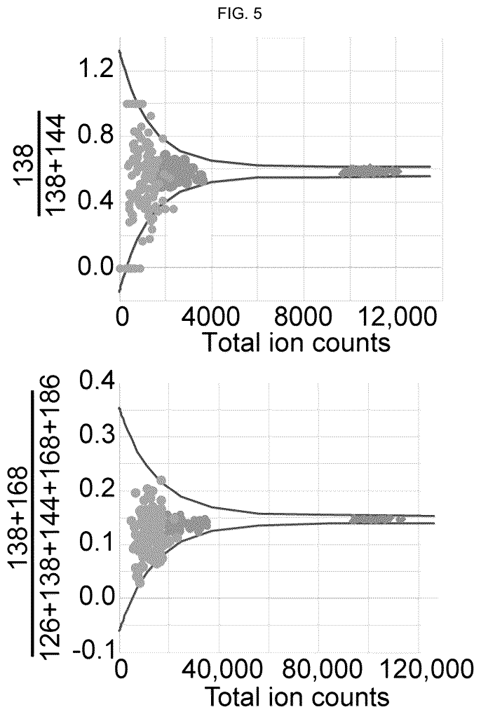

FIG. 5 illustrates an estimation of the error associated with ratios from the total ion counts. A subset of glycans were collected using a range of signal strength to assess the effect of signal quality on the resulting ratios. Two examples are shown for Gal(b1-3)GalNAc. Red lines indicate the exponential functions used to approximate the error as a function of total ion counts. The more the counts of the ions, the lower the error associated with each ratio.

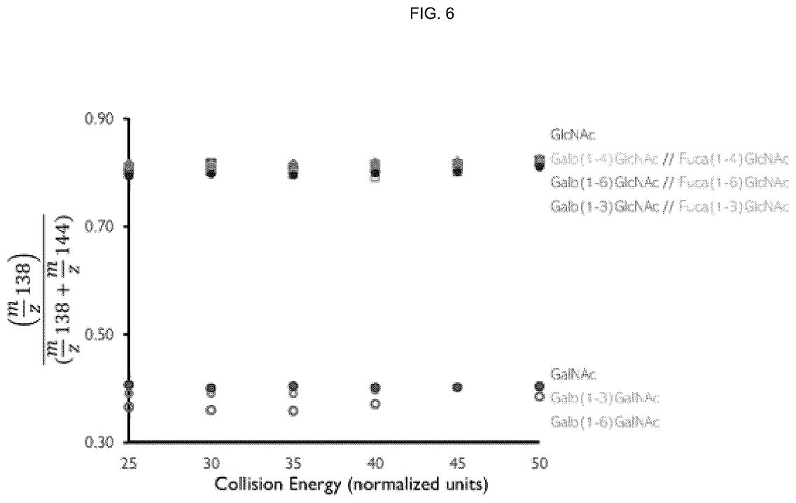

FIG. 6 shows how the ratios of 0.37, 0.81, or 0.73 for (m/z 138)/(m/z 138+m/z 144) point to different structural characteristics of the oligosaccharide portion of a glycan.

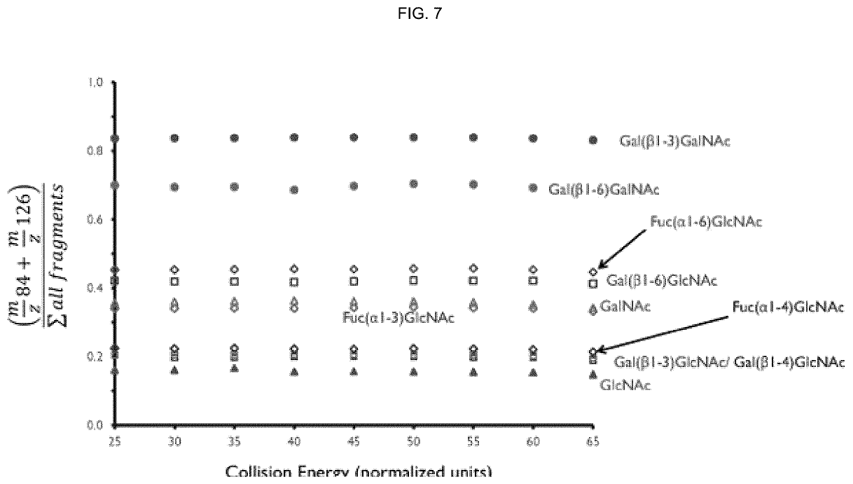

FIG. 7 shows a plot of ratio of MS³ ratio of (m/z 84+m/z 126)/(sum of all m/z 204 fragments) vs collision energy. The plot show that the ratios can differentiate linkage isomers of Gal(β1-x)GalNAc & Fuc(α1-x)GlcNAc.

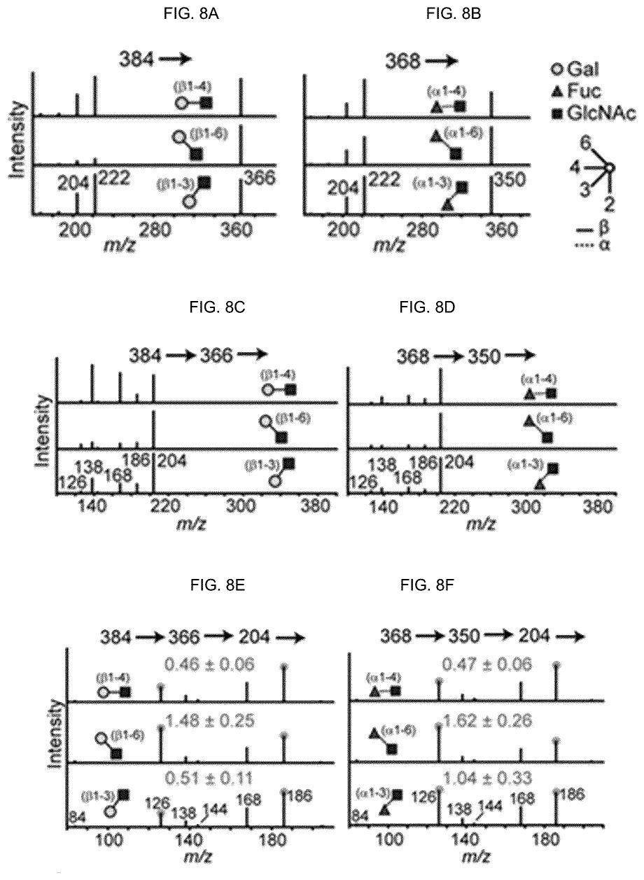

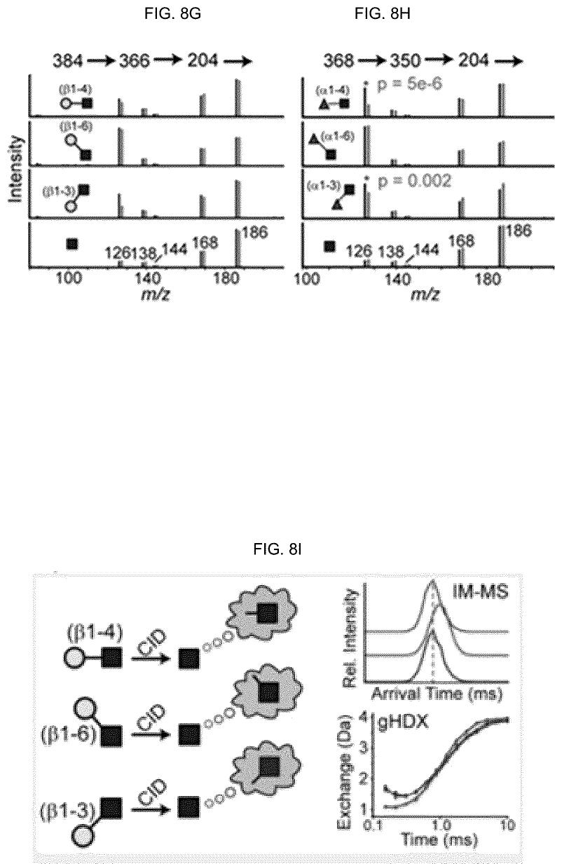

FIGS. 8A to 8I Illustrate examples of MSn spectra of various Gal-GlcNAc and Fuc-GlcNAc disaccharides.

FIG. 8A shows the MSn spectra of Gal(β1-4)GlcNAc (top), Gal(β1-6)GlcNAc (middle), and Gal(β1-3)GlcNAc (bottom) from the MSn−1 peak of m/z 384.

FIG. 8B shows the MSn spectra of Fuc(α1-4)GlcNAc (top), Fuc(α1-6)GlcNAc (middle), and Fuc(α1-3)GlcNAc (bottom) from the MSn−1 peak of m/z 368.

FIG. 8C shows the MSn spectra of Gal(β1-4)GlcNAc (top), Gal(β1-6)GlcNAc (middle), and Gal(β1-3)GlcNAc (bottom) from the MSn−1 peak of m/z 384→366→.

FIG. 8D shows the MSn spectra of Fuc(α1-4)GlcNAc (top), Fuc(α1-6)GlcNAc (middle), and Fuc(α1-3)GlcNAc (bottom) from the MSn−1 peak of 368→350→.

FIG. 8E shows the MSn spectra of Gal(β1-4)GlcNAc (top), Gal(β1-6)GlcNAc (middle), and Gal(β1-3)GlcNAc (bottom) from the MSn−1 peak of 384→366→204→.

FIG. 8F shows the MSn spectra of Fuc(α1-4)GlcNAc (top), Fuc(α1-6)GlcNAc (middle), and Fuc(α1-3)GlcNAc (bottom) from the MSn−1 peak of 368→350→204→.

FIG. 8G shows the anomers of the disaccharides Gal(β1-4)GlcNAc (top), Gal(β1-6)GlcNAc (middle), and Gal(β1-3)GlcNAc (bottom) from the MSn spectra derived from MSn−1 peak of 384→366→204→ were resolved by HILIC and analyzed online by MS4.

FIG. 8H shows the anomers of the disaccharides Fuc(α1-4)GlcNAc (top), Fuc(α1-6)GlcNAc (middle), and Fuc(α1-3)GlcNAc (bottom) from the MSn spectra derived from MSn−1 peak of 368→350→204→ were resolved by HILIC and analyzed online by MS4.

FIG. 8I shows a legend for FIGS. 8A-H. The symbols show the three monosaccharides GlcNAc (in blue), galactose (in yellow), and fucose (in red) that form the six disaccharides, while the solid/dashed lines denote the glycosidic bond between the monosaccharides with the angle of the line specifying a linkage configuration. Solid lines denote a beta (β) linkage while the dashed lines denote the alpha (α) linkage along with the originating carbon atom and ending carbon atom numbered involved in the linkage.

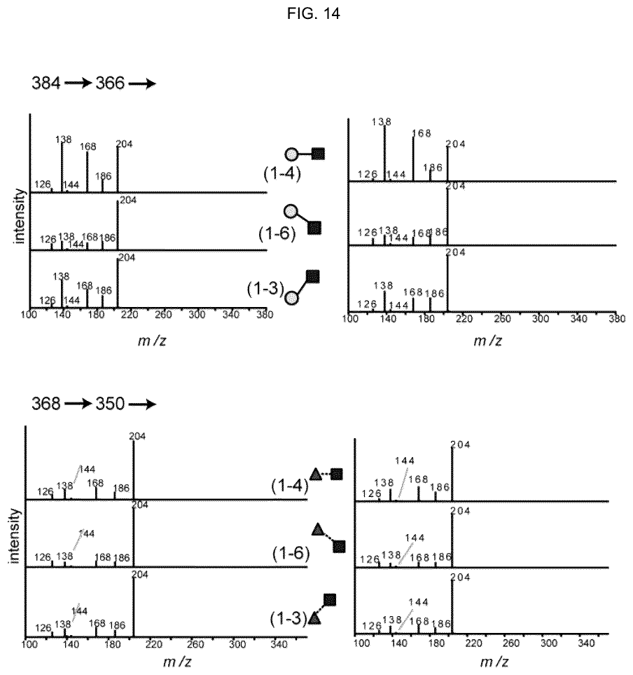

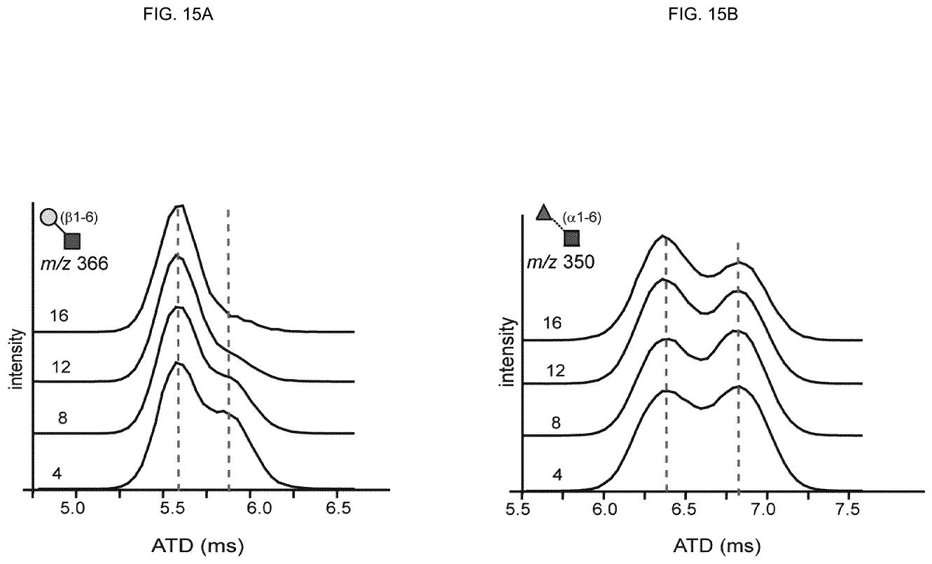

The masses in each panel: m/z 384, 368, 366, 350, and 204 have been selected for each stage of MSⁿ shown by the arrows. Structures of each disaccharide are shown in the panel. The symbols show the three monosaccharides GlcNAc (in blue), galactose (in yellow), and fucose (in red) that form the six disaccharides, while the solid/dashed lines denote the glycosidic bond between the monosaccharides with the angle of the line specifying a linkage configuration (see FIG. 8I). Solid lines denote a beta (β) linkage while the dashed lines denote the alpha (α) linkage along with the originating carbon atom and ending carbon atom numbered involved in the linkage. FIGS. 8G and 8H show that anomers of the disaccharides were resolved by HILIC and analyzed online by MS⁴. The corresponding MS³ data are shown in FIG. 14. The spectra correspond to the intensities of the same ions from the early (black) and late (red) eluting LC peaks. p-values for all comparisons are shown in the inset for FucGlcNAc anomers that produce statistically significant differences in the ratio of the m/z 126/186 ions (all data and comparison matrixes are included in Tables 1 and 2). The GlcNAc monomer analyzed by the same HILIC-MS method is shown on the bottom spectra (222→204→).

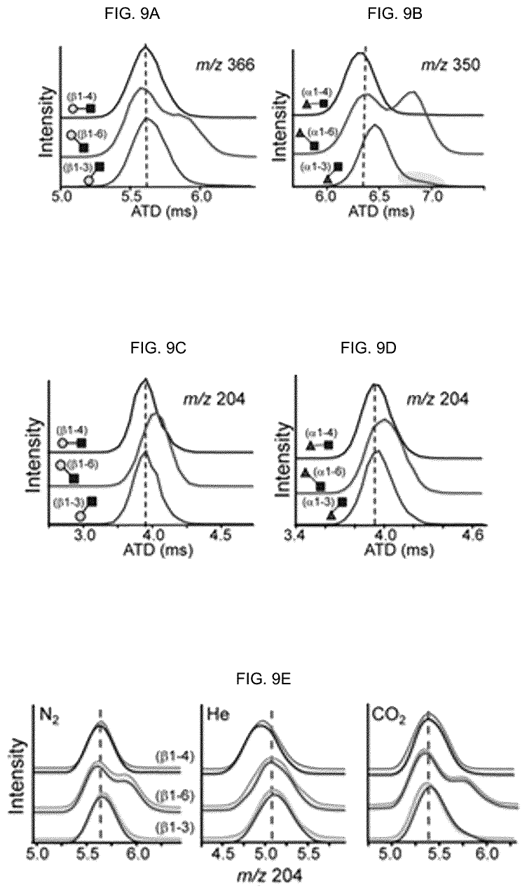

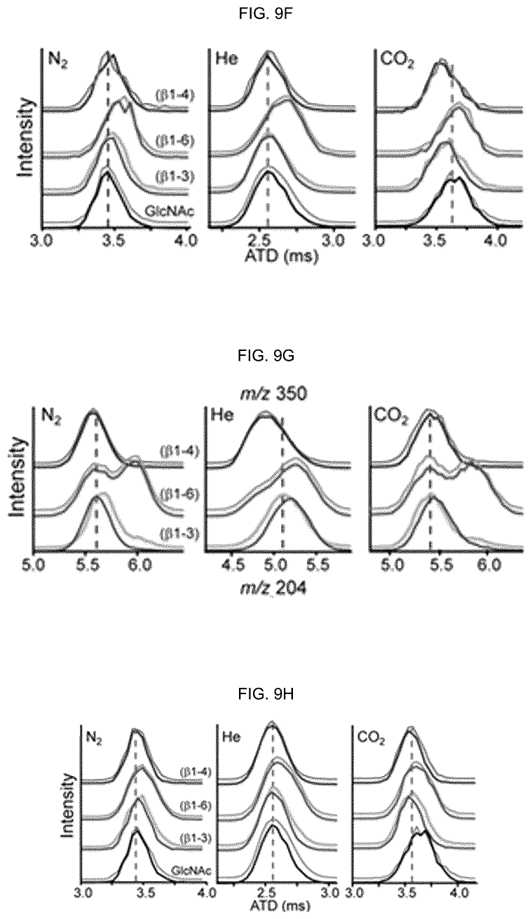

FIGS. 9A to 9H illustrate example arrival time distributions (ATDs) in N₂ of various m/z fractions and the IMMSn analysis of LC resolved anomers of Gal(β1-x)GlcNAc and Fuc(α1-x)GlcNAc.

FIG. 9A shows ATD in N₂ of m/z 366 for Gal(β1-x)GlcNAc.

FIG. 9B shows ATD in N₂ of m/z 350 for Fuc(α1-x)GlcNAc.

FIG. 9C shows ATD in N₂ of m/z 204 from 366 for Gal(β1-x)GlcNAc.

FIG. 9D shows ATD in N₂ of m/z 204 from 350 for Fuc(α1-x)GlcNAc.

FIG. 9E shows the IMMSn analysis of LC resolved anomers of Gal-GlcNAc at ATD of m/z 366.

FIG. 9F shows the IMMSn analysis of LC resolved anomers of Gal(β1-x)GlcNAc at ATD of m/z 204.

FIG. 9G shows the IMMSn analysis of LC resolved anomers of Fuc-GlcNAc at ATD of m/z 350.

FIG. 9H shows the IMMSn analysis of LC resolved anomers of Fuc-GlcNAc at ATD of m/z 204.

N₂, He, and CO₂ were used as the drift gas for FIGS. 9E-9G. The early eluting peak is shown in light blue (1-3), orange (1-6), light green (1-3), and gray (GlcNAc), while the later eluting peak is dark blue, red, dark green, and black. Dashed lines are included to aid in visualizing minor differences in peak positions.

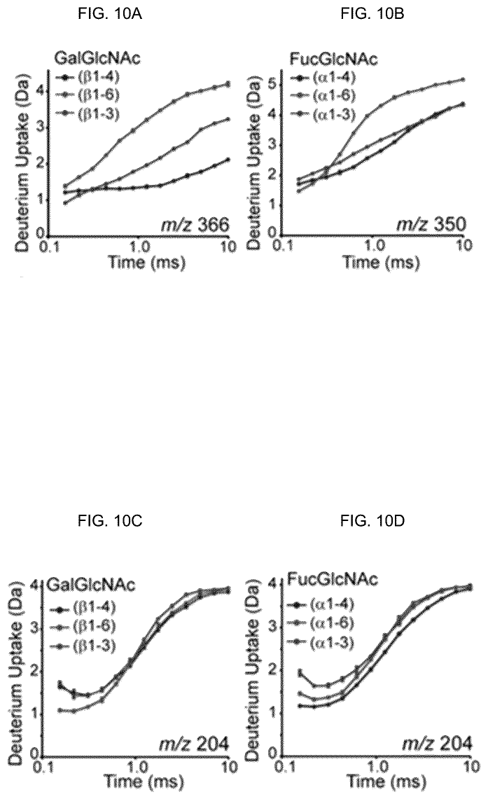

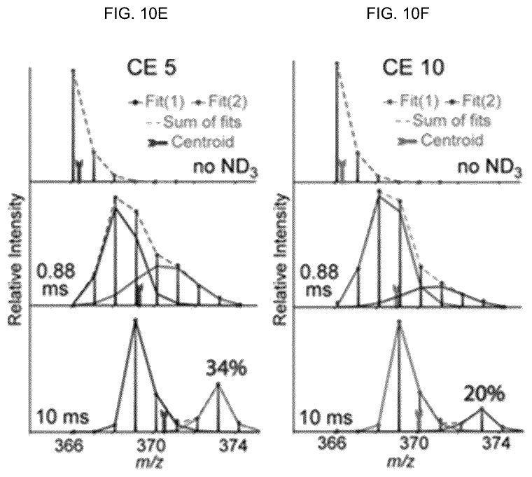

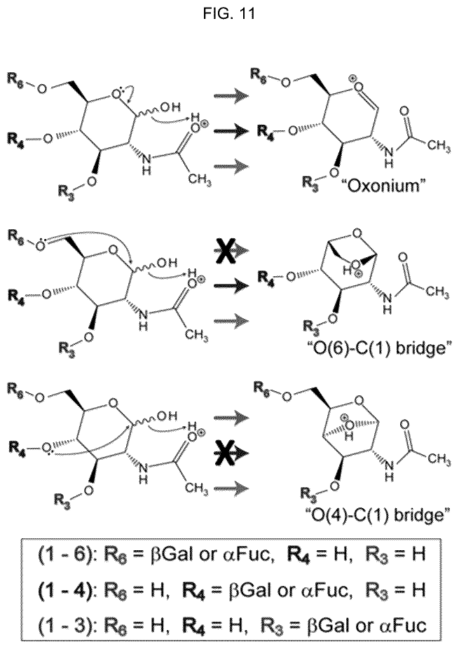

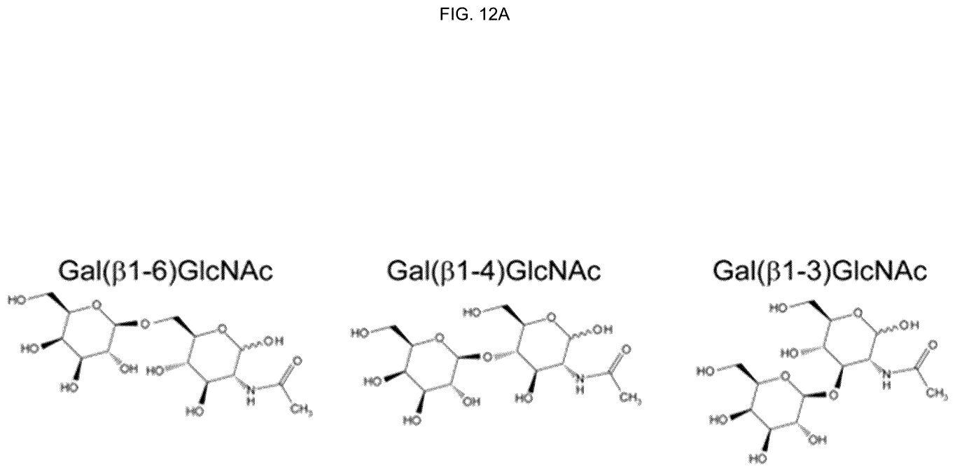

FIGS. 10A-F Illustrate example gHDX analyses of various m/z fractions for Gal(β1-x)GlcNAc and Fuc(α1-x)GlcNAc disaccharides and examples of mass gHDX spectra as a function of collision energy (CE).

FIG. 10A shows gHDX analysis of m/z 366 of Gal(β1-x)GlcNAc disaccharide.

FIG. 10B shows gHDX analysis of m/z 350 of Fuc(α1-x)GlcNAc disaccharide.

FIG. 10C shows gHDX analysis of m/z 204 of Gal(β1-x)GlcNAc disaccharide.

FIG. 10D shows gHDX analysis of m/z 204 of Fuc(α1-x)GlcNAc disaccharide.

FIG. 10E shows an example mass gHDX spectrum from the m/z 366 peak of Gal(β1-6)GlcNAc generated from the precursor ion (m/z 386) using a CE of 5.

FIG. 10F shows an example mass gHDX spectrum from the m/z 366 peak of Gal(β1-6)GlcNAc generated from the precursor ion (m/z 386) using a CE of 10.

Deuterium uptake for the various fragment ions is shown as a function of incubation time with ND3. The points and error bars represent the average and standard deviations from triplicate measurements. The blue and red fits indicate the two populations that are resolved, and the relative abundance of the red population is indicated in the inset.

FIG. 11 illustrates a proposed structural rationalization of linkage memory. GlcNAc linked to another monosaccharide at either the 6 (magenta), 4 (blue), or 3 (green) position undergoes a loss of the reducing end hydroxyl upon fragmentation. The legend at the bottom indicates the positions of the β-Gal or α-Fuc in the disaccharides used in this study. Red arrows indicate potential mechanisms that drive formation of either the classic oxonium structure (top), a O6-C1 linked bridge structure (middle), or a O4-C1 linked bridge structure (bottom). Due to the linkage configuration, the (1-6) linked disaccharides cannot form the O6-C1 bridge structure, and likewise the (1-4) linked disaccharide cannot form the O4-C1 bridge structure (pathways denoted by Xs).

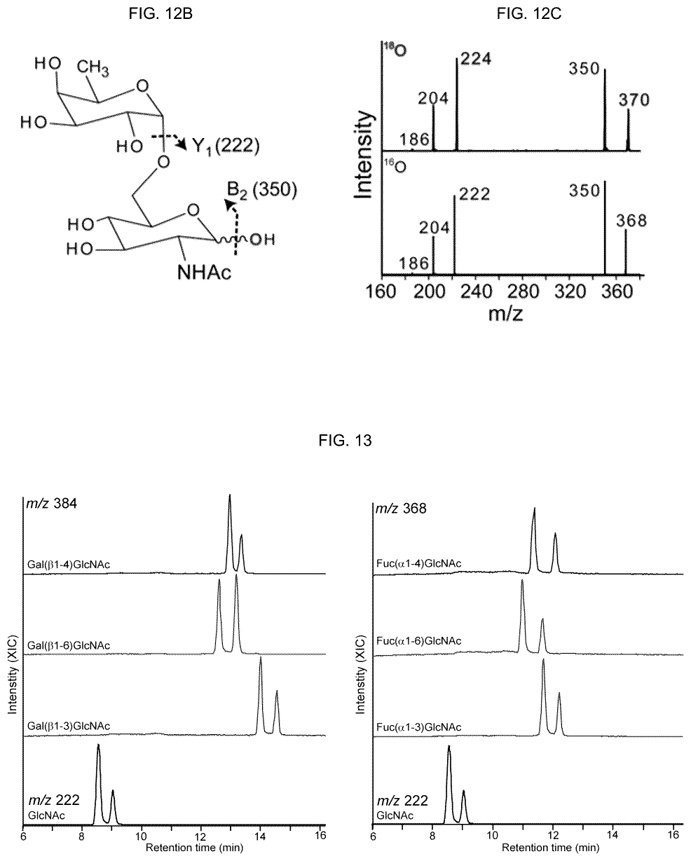

FIGS. 12A to 12C illustrate structures of linkage isomers and fragmentation spectrum of ¹⁸O labeled Fuc-GlcNAc.

FIG. 12A illustrates the chemical structures of the 3 linkage isomers of Gal-GlcNAc.

FIG. 12B illustrates the structure of Fuc(α1-6)GlcNAc showing the primary fragmentations in dashed lines.

FIG. 12C illustrates the CID fragmentation spectrum of Fuc(α1-6)GlcNAc with or without ¹⁸O labeling at the reducing end oxygen (m/z 370 top panel, and 368 bottom panel). Red labels are used for ions containing the ¹⁸O label.

FIG. 13 illustrates separation of α/β anomers of Gal-GlcNAc and Fuc-GlcNAc by HILIC. The two anomers of Gal(β1-x)GlcNAc m/z 384, and Fuc(α1-x)GlcNAc m/z 368 were resolved and analyzed by MS online with HILIC. The extracted ion chromatogram (XIC) traces of each Gal(β1-x)GlcNAc m/z 384, and Fuc(α1-x)GlcNAc m/z 368, where x=3,4,6, are shown along with m/z 222 of GlcNAc monomer.

FIG. 14 illustrates LC-MS³ of the α/β anomers of GalGlcNAc m/z 366 (top panel) and FucGlcNAc m/z 350 (bottom panel) ions. The spectra are tandem mass spectra of the anomers of the Gal(β1-x)GlcNAc m/z 366, and Fuc(α1-x)GlcNAc m/z 350 (where x=3,4, and 6) resolved by HILIC.

FIGS. 15A and 15B illustrate collision energy dependence for the ATDs of Gal(β1-6)GlcNAc m/z 366 and Fuc(α1-6)GlcNAc m/z 350.

FIG. 15A illustrates the ATDs of the Gal(β1-6)GlcNAc m/z 366 at various collision energies from 16 to (top to bottom traces).

FIG. 15B illustrates the ATDs of the Fuc(α1-6)GlcNAc m/z 350 at various collision energies from 16 to (top to bottom traces). With increasing collision energy, there is a decrease in the relative intensity of the species with the larger ATD.

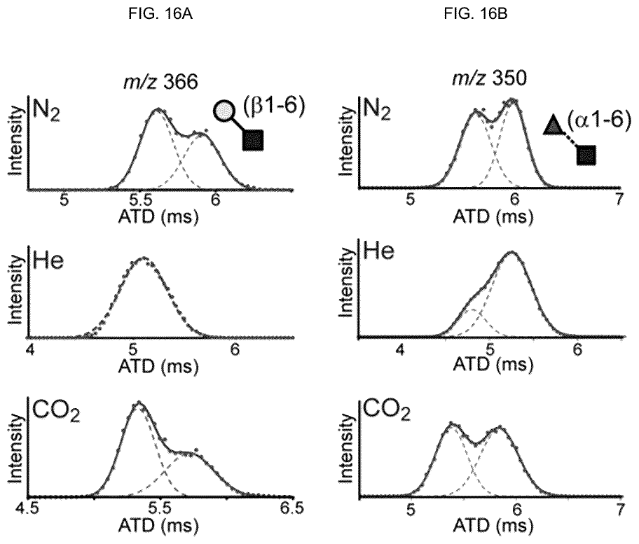

FIGS. 16A and 16B illustrate deconvolution of the multiple arrival time distributions (ATDs) of Gal(β1-6)GlcNAc m/z 366, and Fuc(α1-6)GlcNAc m/z 350.

FIG. 16A illustrates ATD of Gal(β1-6)GlcNAc m/z 366 in N₂, He, and CO₂.

FIG. 16B illustrates ATD of Fuc(α1-6)GlcNAc m/z 350, in N₂, He, and CO₂.

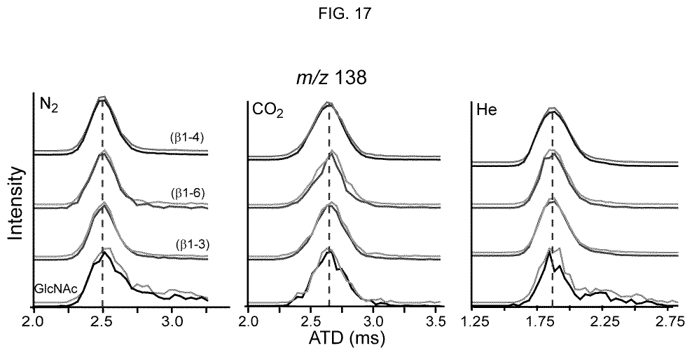

FIG. 17 illustrates ATDs of the m/z 138 ion from each isomer of GalGlcNAc. ATDs of the m/z 138 peak from each dataset of the Gal-GlcNAc and GlcNAc series are shown with the same coloring as used in FIGS. 9E-H. The fact that the m/z 138s are invariant ensures that each dataset was collected under identical conditions.

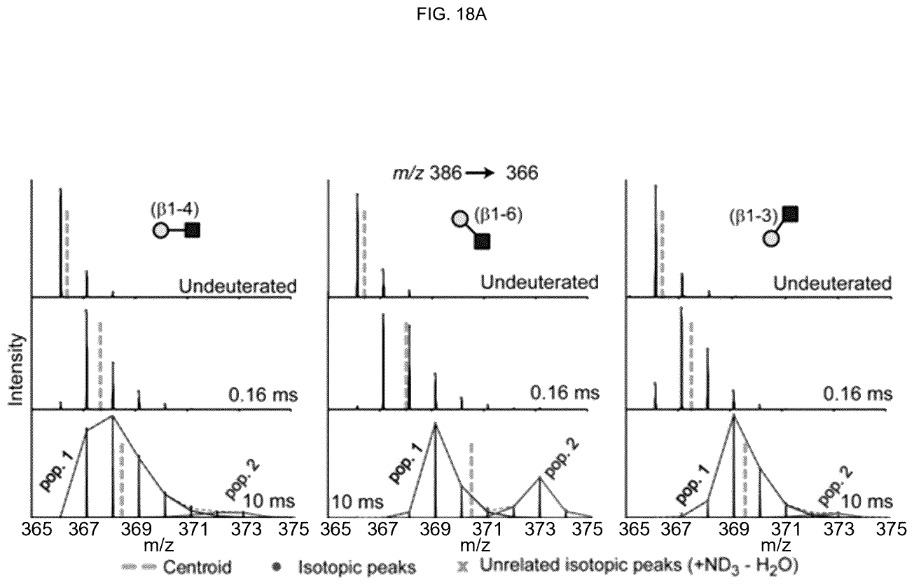

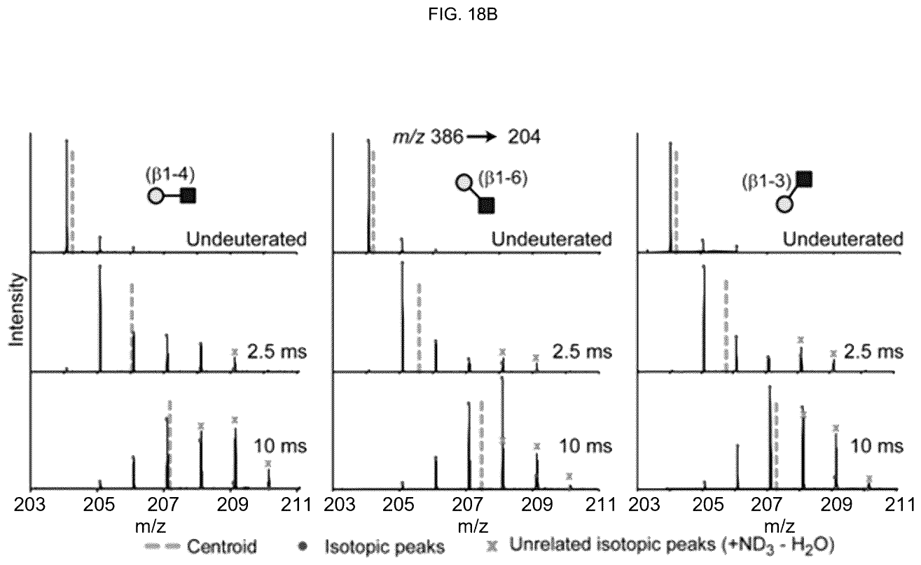

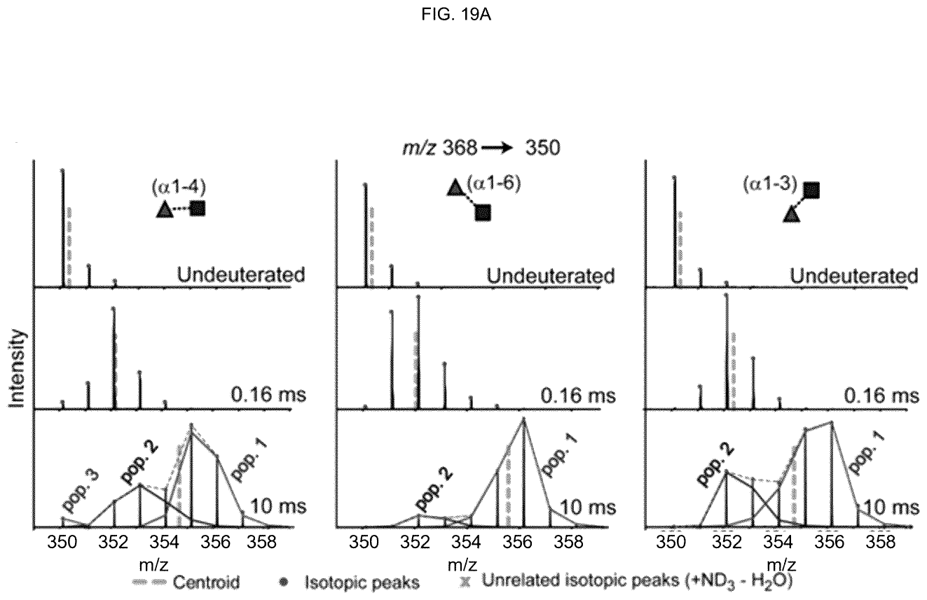

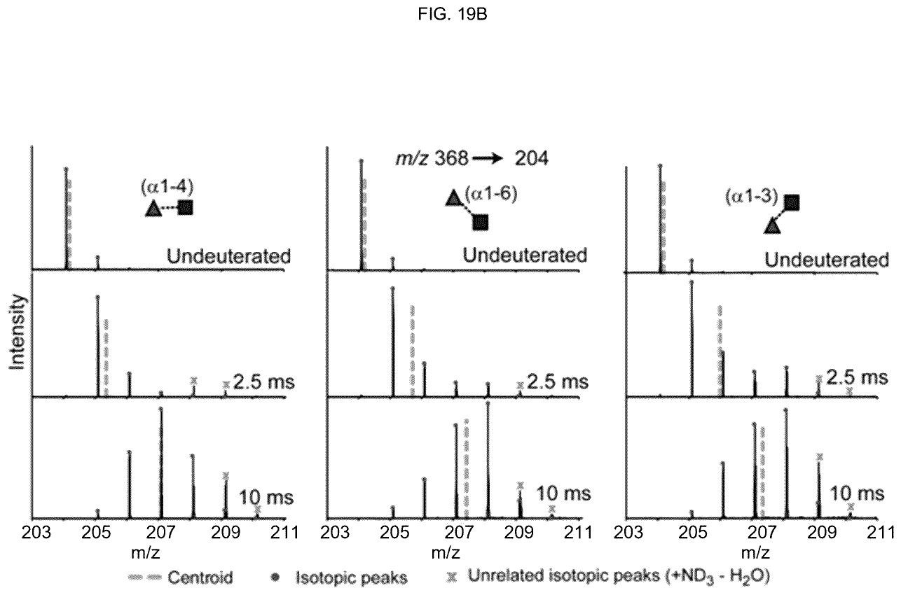

FIGS. 18A and 18B illustrate gHDX spectra of Gal(β1-x)GlcNAc m/z 366 and 204.

FIG. 18A illustrates gHDX spectra of m/z 366 for each of the linkages examined of Gal-GlcNAc.

FIG. 18B illustrates gHDX spectra of m/z 204 for each of the linkages examined of Gal-GlcNAc.

The deuterium exchange time points are indicated in the inset with the undeuterated shown on top. Purple dashed lines indicate positions of centroid of all isotopic peaks (red circles). For the 10 ms time points of the 366 ions the multiple populations (pop.) were fit to a combination of binomial distributions (blue and red fits). Orange x's indicate overlapping unrelated peaks from M+ND₃-H₂O species.

FIGS. 19A to 19B illustrate example gHDX spectra of Fuc(α1-x)GlcNAc m/z 350 and 204.

FIG. 19A illustrates gHDX spectra of m/z 350 for each of the three linkages examined of Fuc-GlcNAc.

FIG. 19B illustrates gHDX spectra of m/z 204 for each of the three linkages examined of Fuc-GlcNAc.

The deuterium exchange time points are indicated in the inset with the undeuterated shown on top. Purple dashed lines indicate positions of centroid of all isotopic peaks (red circles). For the 10 ms time points of the 350 ions the multiple populations (pop.) were fit to a combination of binomial distributions (blue and red fits). In the case of Fuc(α1-4)GlcNAc there was a third population (purple) that did not undergo any gHDX. Orange x's indicate overlapping unrelated peaks from M+ND₃-H₂O species.

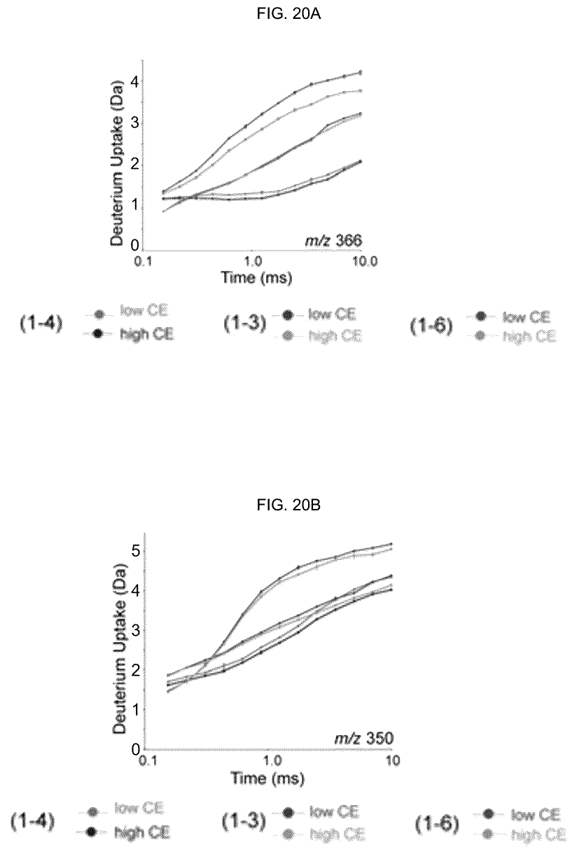

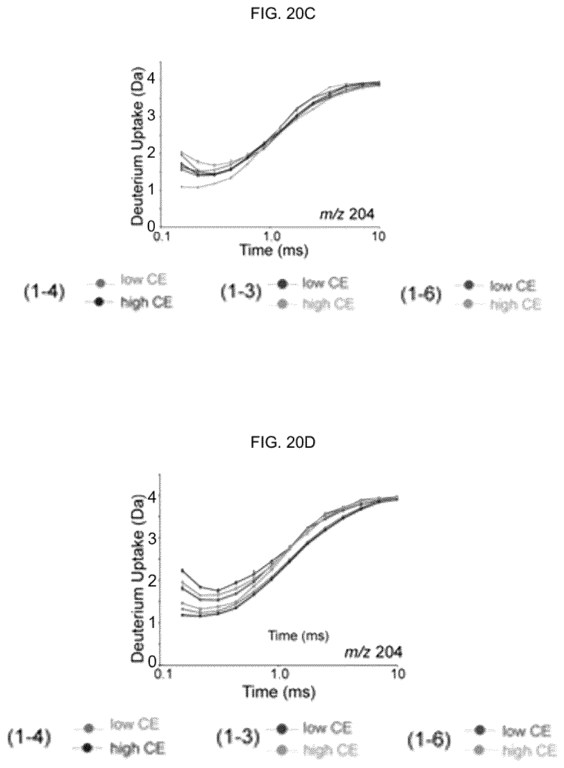

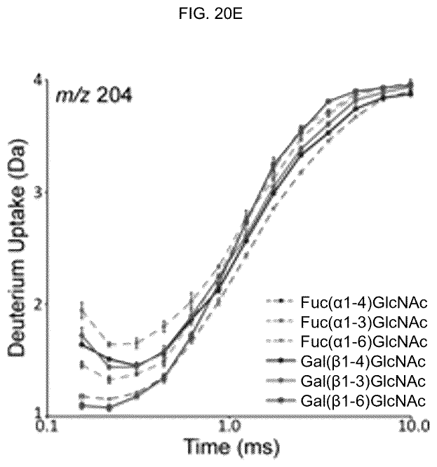

FIGS. 20A to 20E illustrate example collision energy effects on the gHDX kinetics of the m/z 366, 350 and 204 fragments.

FIG. 20A illustrates a deuterium uptake plot of m/z 366 of Gal-GlcNAc.

FIG. 20B illustrates a deuterium uptake plot of m/z 350 of Fuc-GlcNAc.

FIG. 20C illustrates a deuterium uptake plot of subsequent m/z 204 ions from GalGlcNAc.

FIG. 20D illustrates a deuterium uptake plot of subsequent m/z 204 ions from FucGlcNAc.

FIG. 20E illustrates an overlay of the deuterium uptake for the m/z 204 ion from the six different disaccharides at the high CE energies.

Experiments are performed under different collision energies (CEs). For m/z 366 and 350, low CE=5 eV and high CE=10 eV, for m/z 204 from m/z 366 low CE=10 and high CE=17, and for m/z 204 from m/z 350 low CE=10 eV and high CE=22 eV. Error bars represent standard deviation from triplicate measurements.

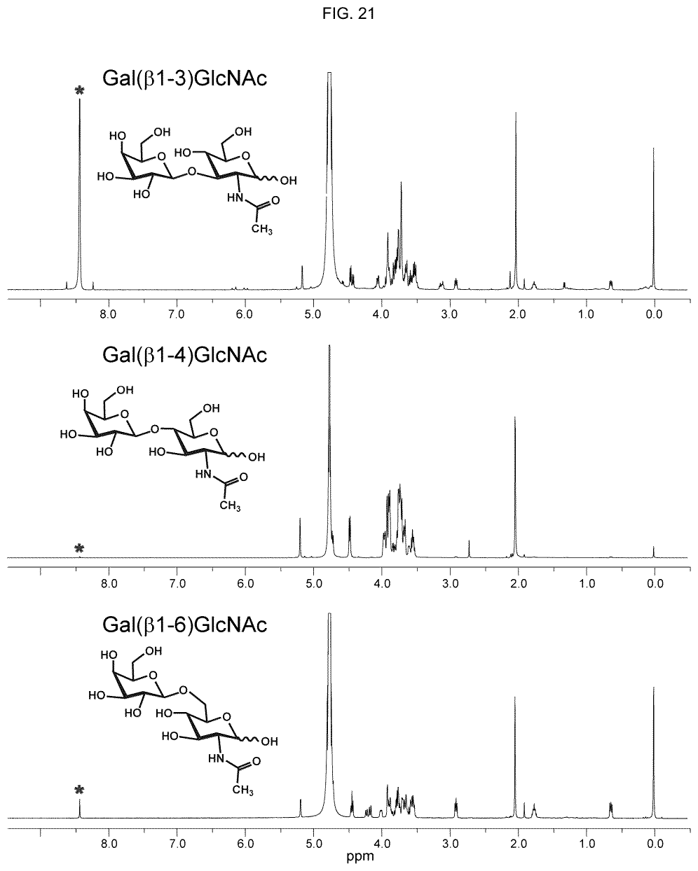

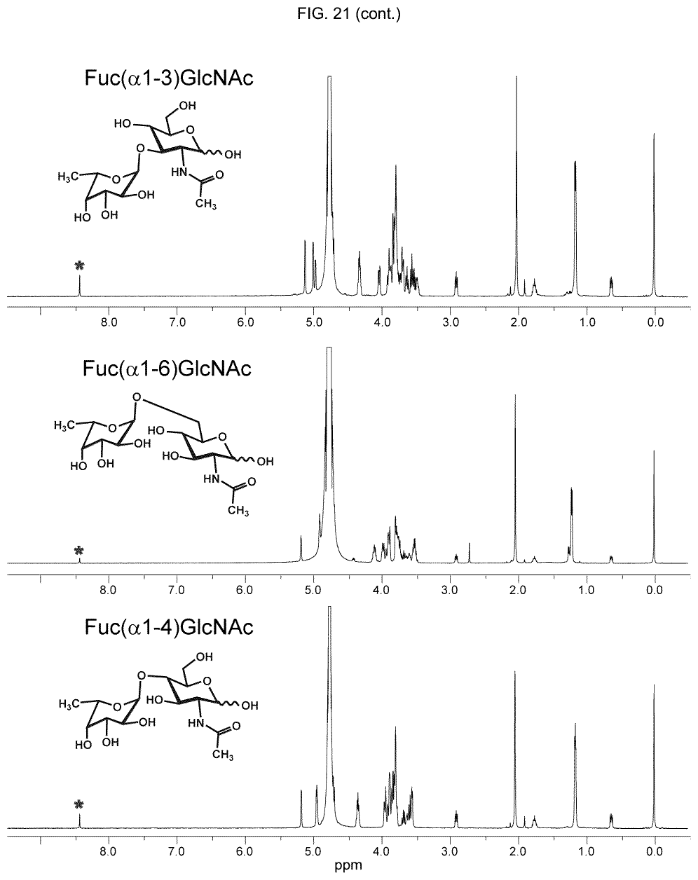

FIG. 21 illustrates NMR spectra of Fuc-GlcNAc and Gal-GlcNAc disaccharides. The peak from the residual formic acid is depicted with a red asterisk.

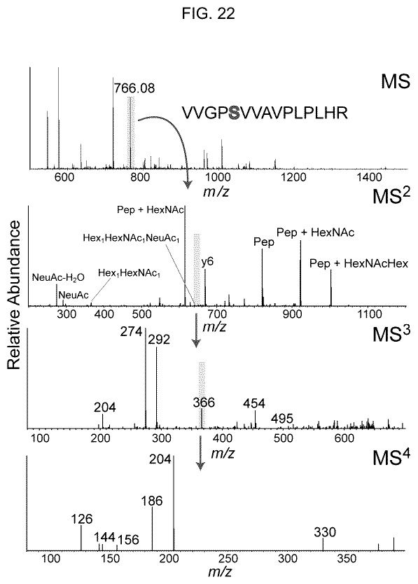

FIG. 22 shows an overview of an example of the LC-MSⁿ approach applied to an actual glycopeptide from a glycoprotein digest (indicated in the top inset with glycosylated serine in purple). MS2 using CID produces several neutral losses and a 657 and 366 ion corresponding to di and trisaccharide ions. MS³ of the 657 produces sufficient signal of the fragments to calculate the ion ratios. MS⁴ of the 366 (from the 657) is still able to detect the fragmentation pattern albeit with weaker overall counts.

FIG. 23 illustrates the formation of ethyl glucoside by the combination of glucose and ethanol (with water as a byproduct).

FIG. 24 illustrates the overview MSⁿ for obtaining maximum structural information on glycopeptides. Peptide sequence and glycan linkage is obtained from MS² spectra. Further details about the glycan structure including specific linkage configurations and monosaccharide identity are obtained from MS³ and MS⁴ spectra.

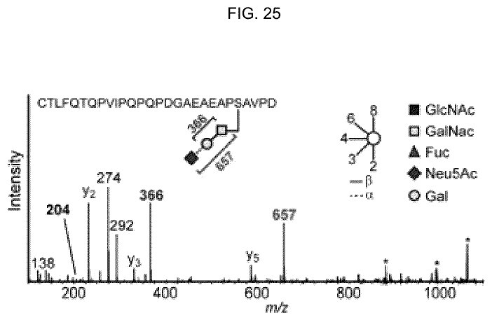

FIG. 25 illustrates example spectra of an O-linked glycopeptide after fragmentation. The 138, 204, 274, 292, 366 and 657 ions are carbohydrates B-type ions that are often abundant. The peptide sequence and structure is shown on the top using the carbohydrate symbol nomenclature depicted on the top right. * indicate neutral loss fragments that are used to decipher glycan composition.

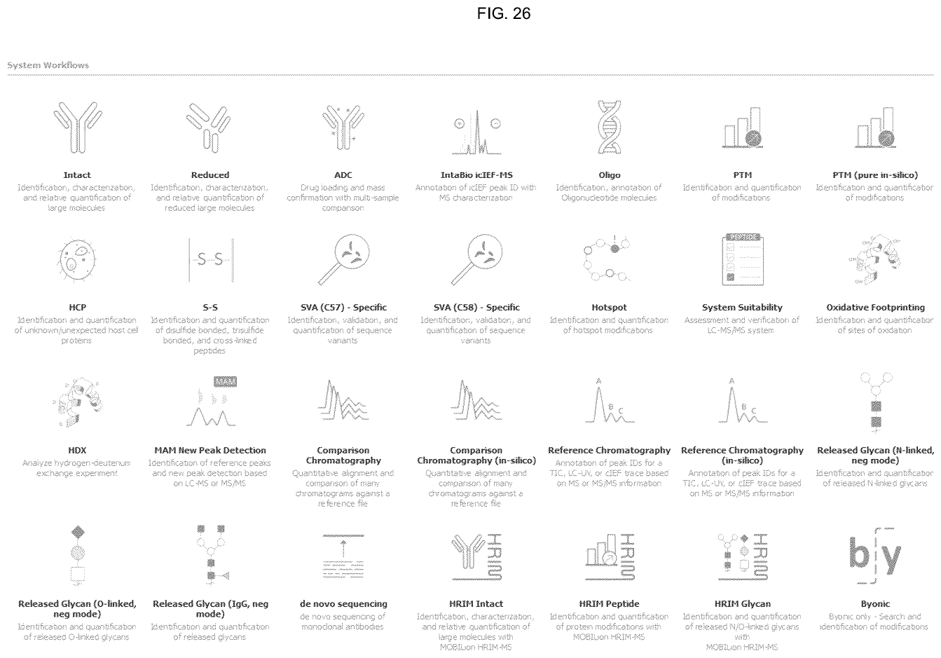

FIG. 26 illustrates that Protein Metrics' Byos platform that offers about 25 predefined workflows, which prompt the users for required inputs and parameter settings, and then launch the software modules to run the data on the desktop or in the Cloud.

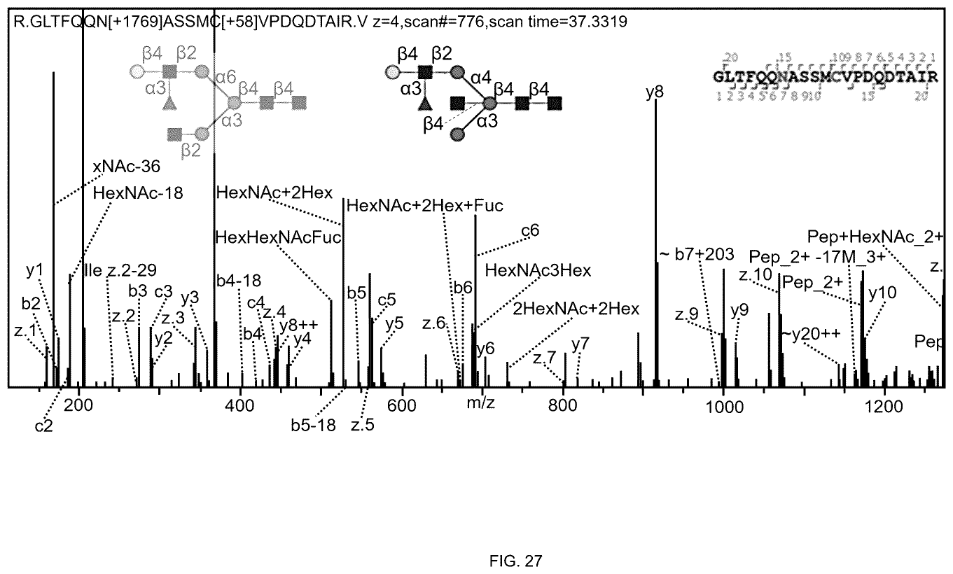

FIG. 27 illustrates that Byonic annotates glycopeptides MS/MS spectra including diagnostic carbohydrate peaks. The fragment ion at 690 indicates the presence of a bisecting GlcNAc (dark inset structure on the right) rather than G2F (shown with the faint inset structure on the left), a common glycan on IgG antibodies. Hex₁HexNAc₁Fuc₁ at 512 indicates the presence of antennal fucose. The solid structure in the inset shown is consistent with the spectrum, but cannot resolve potential linkage configurations. MSⁿ on peaks at 366 and 512 can resolve these linkage possibilities.

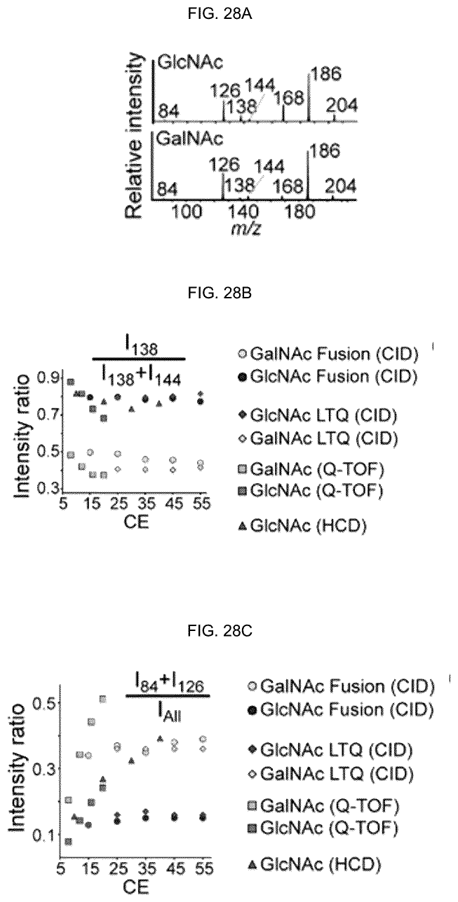

FIGS. 28A to 28C illustrate the benefits of ion trap wherein MSⁿ is used for monosaccharide differentiation.

FIG. 28A illustrates ion trap CID MS/MS spectra of the m/z 204 ion from GlcNAc and GalNAc.

FIG. 28B illustrates the intensity ratio of m/z 138 compared to the sum of 138 and 144 collected at different collision energies (CE).

FIG. 28C illustrates the intensity ratio of m/z 84 and 126 compared to the sum of all fragments of m/z 204 collected at different collision energy (CE). Data for GlcNac and GalNAc are shown collected using different types of collision cells (Q-TOF, HCD, and CID on both a Thermo Fusion and LTQ instrument).

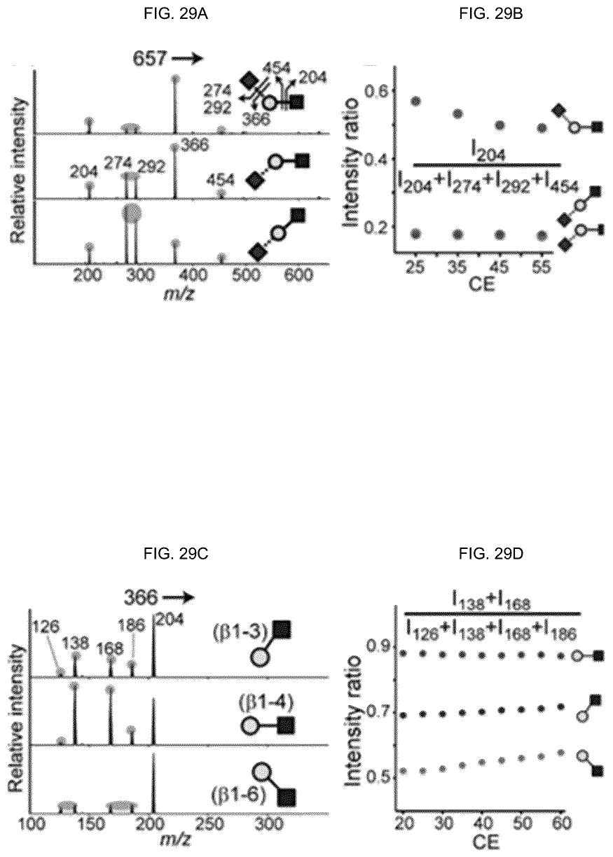

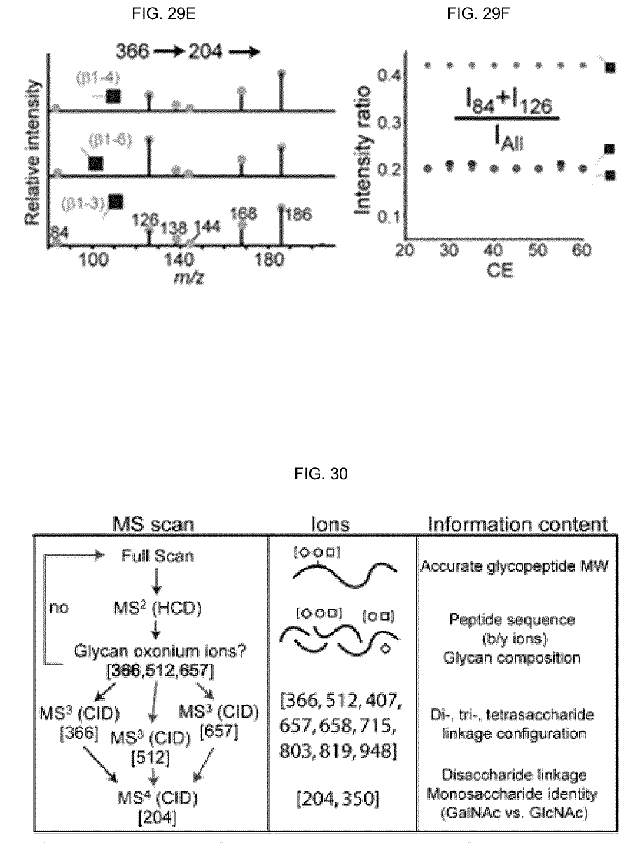

FIGS. 29A to 29F illustrate ion trap CID fragment propensity examples from various di- and trisaccharides. Structures are denoted with the symbol nomenclature shown in FIG. 25.

FIG. 29A illustrates MS³ fragment spectra of the m/z 657 ion from three isomers of Neu5Ac-Gal-GlcNAc. Ions of interest are highlighted.

FIG. 29B illustrates the ratio of the intensity of m/z 204: (204+274+292+454) that is plotted at different CE. The (α2-6) linked Neu5Ac species (purple) shows a very distinct higher ratio than the isomers containers (α2-3) linkages.

FIG. 29C illustrates MS³ fragment spectra of the m/z 366 ion from the isomers of Gal-GlcNAc.

FIG. 29D illustrates that the ratio of the intensity of m/z (138+168): (126+138+168+186) reveals a characteristic ratio for each linkage isomer that is not significantly influenced by CE.

FIG. 29E illustrates MS⁴ fragment spectra of m/z 204 that is isolated from the three linkage isomers in FIG. 29C.

FIG. 29F illustrates that the intensity ratios of the m/z (84+126): (all 204 fragment ions) is characteristically different from the (α1-6) linkage isomer. Additionally, the m/z 138 and 144 ratios are also present to differentiate GlcNAc and GalNAc as outlined in FIG. 28.

FIG. 30 provides an overview of the ions of interest and information content obtained from each stage of MS. Example carbohydrate ions are shown in brackets. The MS acquisition protocol is shown on the left. MS² scans are collected in an untargeted data-dependent fashion. Any MS² scans showing glycan oxonium fragment ions (e.g., m/z 366, 512, 657) are then programmed to perform subsequent CID MS³ and MS⁴ scans from the same precursor glycopeptide.

The present disclosure provides a method of analyzing the structure of a glycan sample, the method including:
receiving data indicative of one or more spectra of mass-to-charge ratio (m/z) versus relative abundance of the glycan sample from a mass spectrometer (MS) instrument;
generating a ratio according to Equation 1, wherein a is a magnitude of one or more first peaks in the one or more spectra and b is the magnitude of one or more second peaks in the one or more spectra;
determining that the ratio is within a range of a predetermined ratio;
based on determining that the ratio is within the range of the predetermined ratio, determining that a predetermined structural characteristic is present in the glycan sample; and
outputting an indication of the predetermined structural characteristic in the glycan sample.

In implementations, the glycan sample includes a protonated glycan including an oligosaccharide. A protonated glycan includes an O-glycan or an N-glycan.

N-glycans are covalently attached to protein at asparagine (Asn) residues by an N-glycosidic bond. A common N-glycan linkage is the attachment of N-acetylglucosamine to asparagine (GlcNAcβ1-Asn). An example of such an N-glycan is: GlcNAcβ(1-2)Manα(1-3)[GlcNAcβ(1-2)Manα(1-6)]Manβ(1-4)GlcNAcβ(1-4)GlcNAc-Asn, which has the structure:

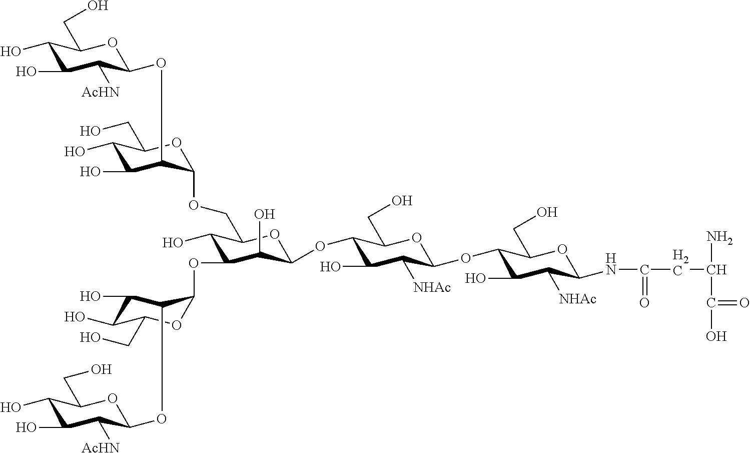

Various examples of O-glycans have N-acetylgalactosamine (GalNAc) as a common core, although other cores are also possible. O-glycans result from O-glycosylation that is a common covalent modification of serine and threonine residues of mammalian glycoproteins. In mucins, O-glycans can be covalently α-linked via an N-acetylgalactosamine (GalNAc) moiety to the —OH of serine or threonine by an O-glycosidic bond, and the structures are named mucin O-glycans or O-GalNAc glycans. An example of an O-glycan is Galβ(1-3)GalNAc-α-1-O-Serine (also represented as H-Ser(Gal(β(1-3)GalNAc)-OH) having the structure:

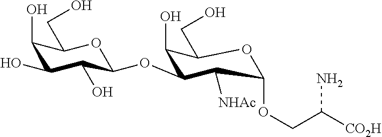

Another example of an O-glycan is O-[2-Acetamido-2-deoxy-3-O-(β-D-galactopyranosyl)-α-D-galactopyranosyl]-L-threonine, having the structure:

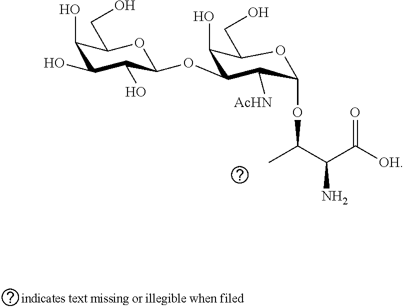

In implementations, the protonated glycan of this disclosure includes a sialylated glycan.

In implementations, the glycan of this disclosure includes an oligosaccharide, and wherein the monosaccharides of the oligosaccharide are linked through O-glycosidic linkages.

In implementations, the predetermined structural characteristic includes:
an identity of the one or more monosaccharides or oligosaccharides of the glycan;
a connectivity of the O-glycosidic linkages of the one or more oligosaccharides of the glycan; or
a configuration of anomeric carbons in the one or more monosaccharides or oligosaccharides of the glycan.

In implementations, the connectivity of the O-glycosidic linkages can, for example, be (1-2), (1-3), (1-6) positional linkages connecting two monosaccharides of an oligosaccharide of the glycan. As an example, using Fucose and GlcNAc as the two monomers of a disaccharide, these linkages can be represented structurally as:

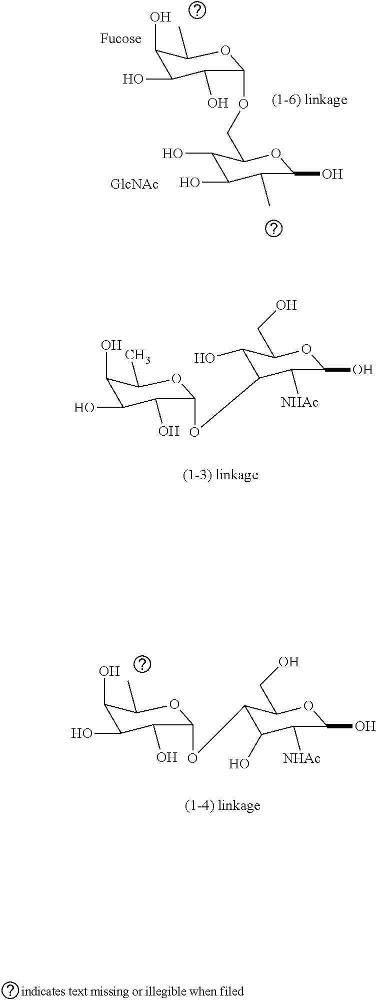

The configuration of the anomeric carbons of each monomer can be a (alpha) or β (beta), and the O-glycosidic linkage can be α or β.

As an example, FIG. 3 shows examples of the inherent structural heterogeneity of glycans, showing structural diversity in terms of composition, the connectivity of O-glycosidic linkages and the configuration of anomeric carbons (α vs β) in an oligosaccharide of the glycan.

In implementations, the predetermined ratio of the disclosure has a variability (uncertainty; ratio error) associated with it, which is based on the number of ion counts associated with the one or more spectra. Accordingly, the presently disclosed method of analyzing the structure of a glycan sample further includes determining the variability of the predetermined ration based on a number of ion counts associated with the one or more spectra.

As set forth in FIG. 5, the more counts of the ions, the lower the error associated with each ratio. Using the kinds of data sets shown in this Figure, it is possible to approximate the variability from a single measurement using the total ion counts within the spectra.

In implementations, the presently disclosed method of analyzing the structure of a glycan sample further includes:
obtaining, by the MS instrument, the one or more spectra by passing the glycan sample through the MS instrument, and
generating, by one or more analog-to-digital converters (ADCs), the data indicative of one or more spectra.

In implementations, obtaining, by the MS instrument, the one or more spectra includes: obtaining, by the MS instrument, a first MS spectrum (MS¹ spectrum) by passing the glycan sample through the MS instrument a first time;
obtaining a portion of the carbohydrate sample corresponding to one or more peaks of the MS¹ spectrum; and
obtaining, by the MS instrument, a second MS spectrum (MS² spectrum) by passing the portion of the carbohydrate sample through the MS instrument a second time.

In implementations, the portion of the glycan sample referred to immediately above is a first portion of the glycan sample, and
wherein, obtaining, by the MS instrument, the one or more spectra further includes:
   obtaining a second portion of the glycan sample corresponding to one or more peaks of the MS² spectrum; and
      obtaining, by the MS instrument, a third MS spectrum (MS³ spectrum) by passing the second portion of the glycan sample through the MS instrument a third time.

In implementations, obtaining, by the MS instrument, the one or more spectra, referred to immediately above, further includes:
obtaining a third portion of the carbohydrate sample corresponding to one or more peaks of the MS3 spectrum; and
obtaining, by the MS instrument, a fourth MS spectrum (MS4 spectrum) by passing the third portion of the glycan sample through the MS instrument a fourth time.

Accordingly, in implementations, the presently disclosed method of analyzing the structure of a glycan sample involves tandem mass spectrometry, also known as MS/MS (or MS-MS), where two or more mass analyzers are coupled together using an additional reaction step to increase their abilities to analyze chemical samples, such as glycans, proteins, peptides, and other biomolecules.

The molecules of a given sample are ionized and the first spectrometer (designated MS1) separates these ions by their mass-to-charge ratio (often given as m/z or m/Q). Ions of a particular m/z-ratio coming from MS¹ are selected and then made to split into smaller fragment ions, e.g., by collision-induced dissociation, ion-molecule reaction, or photodissociation. These fragments are then introduced into the second mass spectrometer (MS²), which in turn separates the fragments by their m/z-ratio and detects them. Fragments from MS² can also be introduced, into a third mass spectrometer (MS³), and fragments from MS³ can also be introduced, for structure elucidation of the sample into a fourth mass spectrometer (MS⁴) for additional information. In some examples, the same spectrometer device performs each MSⁿ round, where n is a positive integer. The fragmentation steps make it possible to identify and separate ions that have very similar m/z-ratios in regular mass spectrometers.

In implementations, prior to obtaining by the MS instrument, the one or more spectra by passing the glycan sample through the MS instrument, the glycan sample is passed through a liquid chromatography (LC) instrument, and a portion of the glycan sample corresponding to a particular peak in the liquid chromatogram is obtained and passed through the MS instrument. Accordingly, this method employs what is commonly known as liquid chromatography with tandem mass spectrometry (LC-MS-MS), which is a powerful analytical technique that combines the separating power of liquid chromatography with the highly sensitive and selective mass analysis capability of triple quadruple mass spectrometry.

In implementations, in the presently disclosed method of analyzing the structure of a glycan sample, the one or more first peaks are defined at an m/z of 138 and the one or more second peaks are defined at an m/z of 144 in an MSⁿ spectrum derived from fragmentation of a portion of the sample corresponding to m/z 204 in an MSⁿ⁻¹ spectrum,
wherein the predetermined ratio is 0.37, 0.81, or 0.73 and
wherein the predetermined structural characteristic includes:
   the presence of an isomer in the glycan sample, the isomer including N-acetylgalactosamine (GalNAc), N-acetylglucosamine (GlcNAc), or N-acetylmannosamine (ManNAc); or
   the presence at the reducing end of an oligosaccharide in the glycan sample, of GalNAc or GlcNAc.

N-acetylgalactosamine (GalNAc), for instance, has the structure:

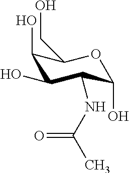

and in Haworth projection

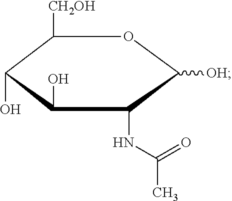

N-acetylglucosamine (GlcNAc) has the structure:

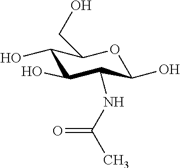

and in Haworth projection

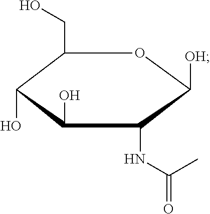

N-acetylmannosamine (ManNAc) has the structure:

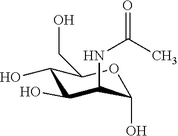

and in Haworth projection

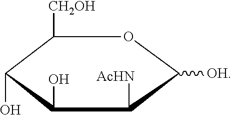

As used herein, “the reducing end” of a sugar refers to the sugar structure with a free aldehyde or ketone group. The end of the molecule with the free anomeric carbon is referred to as the reducing end.

Thus, in implementations, the ratio (m/z 138)/(m/z 138+m/z 144) from the fragmentation of m/z 204 may distinguish between GalNAc, GlcNAc & ManNAc—composition isomers having same m/z but differ at one stereochemistry of hydroxyl at one carbon. Thus, GalNAc and GlcNAC differ in the configuration of the anomeric carbon, whereas GlCNAc and ManNAC differ in the configuration of the carbon atom bearing the N-acetyl group. This ratio can also be used to assess whether larger carbohydrates (e.g., tri/tetra-saccharides) have GalNAc or GlcNAc on their reducing end.

FIG. 6 shows how the ratios of 0.37, 0.81, or 0.73 for (m/z 138)/(m/z 138+m/z 144) point to different structural characteristics of the oligosaccharide portion of a glycan. Thus, for example, the ratio of 0.37 indicates the presence of Gal(β1-3)GalNAc and Gal(β1-6)GalNAc (in both these disaccharides, GalNAc is the reducing end of the disaccharide), whereas the ratios of 0.73 and 0.81 indicate the presence of Gal(β1-4)GlcNAc or Fuc(α1-4)GlcNAc; Gal(β1-6)GlcNAc or Fuc(α1-6)GlcNAc; or Gal(β1-3)GlcNAc or Fuc(α1-3)GlcNAc (in these disaccharides, GlcNAc is the reducing end of the disaccharide). Fuc is fucose, having the structure:

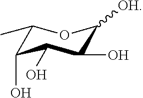

As an illustration, the structure of Gal(β1-3)GalNAc is:

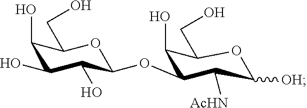

the structure of Gal(β1-4)GlcNAc is:

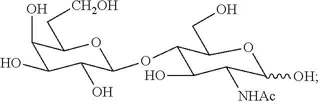 [SMILES: CC(=O)NC1C(O)OC(CO)C(OC2OC(CCO)C(O)C(O)C2O)C1O]

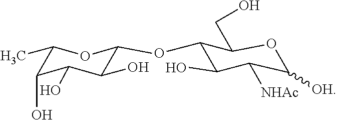 [SMILES: CC(=O)NC1C(O)OC(CO)C(OC2OC(C)C(O)C(O)C2O)C1O]

and the structure of Fuc(α1-4)GlcNAc is:

In implementations, the ratios of 0.37, 0.81, or 0.73 for (m/z 138)/(m/z 138+m/z 144) all have variabilities associated with them. In implementations, the variability associated with the ratio of 0.37 is ±0.05. In implementations, the variability associated with the ratio of 0.81 is ±0.04. In implementations, the variability associated with the ratio of 0.73 is ±0.11.

In implementations, in the presently disclosed method of analyzing the structure of a glycan sample, the one or more first peaks are defined at an m/z of 84 and 126 and the one or more second peaks are defined at an m/z of 138, 144, 168, and 186 in an MSⁿ spectrum derived from fragmentation of a portion of the sample corresponding to m/z 204 in an MSⁿ⁻¹ spectrum,
wherein the predetermined ratio is 0.21, 0.20, or 0.42 and
wherein the predetermined structural characteristic includes:
a type of a O-glycosidic linkage between two monosaccharides in a disaccharide portion of the glycan sample, the type of the O-glycosidic linkage being 1-3, 1-4, or 1-6 linkage.

In implementations, the two monosaccharides include: Fucα and GlcNAc; or Galβ and GlcNAc. This is illustrated in FIG. 7, wherein Gal(β1-3)GalNAc appears at a ratio of 0.20, Fuc(α1-4)GlcNAc appears at a ratio of 0.21, and Gal(β1-6)GlcNAc and Fuc(α1-6)GlcNAc appear at a ratio of 0.42

In implementations, in the presently disclosed method of analyzing the structure of a glycan sample, wherein the one or more first peaks are defined at an m/z of 138 and 168, and the one more second peaks are defined at an m/z of 126, 144, and 186 in an MSⁿ spectrum derived from fragmentation of a portion of the sample corresponding to an m/z of 366 in an MSⁿ⁻¹ spectrum,
wherein the predetermined ratio is 0.621, 0.843, or 0.536 and
wherein the predetermined structural characteristic includes:
   a type of a O-glycosidic linkage between two monosaccharides in an oligosaccharide portion of the glycan sample, the type of the O-glycosidic linkage being 1-3, 1-4, or 1-6 linkage.

In implementations, the two monosaccharides include Galβ and GlcNAc; or Fucβ and GlcNAc. Thus, for example, Gal(β1-6)GlcNAc appears at a ratio of 0.536; Neu5Ac(α2-3)Gal(β1-3)GlcNAc(β1-3)Glc appears at a ratio of 0.621; and Gal(β1-6)GlcNAc appears at a ratio of 0.536. Neu5Ac is N-Acetylneuraminic acid (also known as NANA), and has the structure:

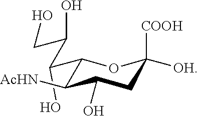

In implementations, the ratios of 0.621, 0.843, or 0.536 for (m/z 138+m/z 168)/(m/z 138+m/z 168+m/z 126+m/z 144) all have variabilities associated with them. In implementations, the variability associated with the ratio of 0.621 is ±0.012. In implementations, the variability associated with the ratio of 0.843 is ±0.007. In implementations, the variability associated with the ratio of 0.536 is ±0.02.

In implementations, in the presently disclosed method of analyzing the structure of a glycan sample, the one or more first peaks are defined at an m/z of 186 and 204, and the one or more second peaks are defined at an m/z of 256, 274, 292, 454, and 495 in an MSⁿ spectrum derived from fragmentation of a portion of the carbohydrate sample corresponding to an m/z of 657 in an MSⁿ⁻¹ spectrum;
wherein the predetermined ratio is 0.426 or 0.219, and
wherein the predetermined structural characteristic includes:
   a type of O-glycosidic linkage between two monosaccharides of a disaccharide portion of the glycan sample, the type of the glycosidic bond linkage being an (α2-6) or an (α2-3) linkage.

In implementations, one of the two monosaccharides is Neu5Ac. In implementations, one of the two monosaccharides in Neu5Ac, and the other is Gal. Thus for example, Neu5Ac(α2-6)Gal(β1-4)GlcNAc appears at a ratio of 0.426, and Neu5Ac(α2-3)Gal(β1-4)GlcNAc appears at a ratio of 0.219.

In implementations, the ratios of 0.426 and 0.219 for (m/z 186+m/z 204)/(m/z 186+m/z 204+m/z 256+m/z 274+m/z 292+m/z 454+m/z 495) all have variabilities associated with them. In implementations, the variability associated with the ratio of 0.426 is ±0.03. In implementations, the variability associated with the ratio of 0.219 is ±0.007.

In implementations, in the presently disclosed method of analyzing the structure of a glycan sample, the one or more first peaks are defined at an m/z of 204 and 366, and the one or more second peaks are defined at an m/z of 274, 292, 454, 472 and 495 in an MSⁿ spectrum derived from fragmentation of a portion of the carbohydrate sample corresponding to an m/z of 657 in an MSⁿ⁻¹ spectrum,
wherein the predetermined ratio is 0.867, 0.758, 0.685, 0.536, 0.531, 0.403, or 0.234, and
wherein the predetermined structural characteristic includes:
   a type of O-glycosidic linkage between Neu5Ac and Gal in a disaccharide portion of the glycan sample, the type of the glycosidic linkage being an (α2-6) or an (α2-3) linkage;
   a type of O-glycosidic linkage between Gal and GlcNAc in a tetrasaccharide portion of the glycan sample, the type of the glycosidic linkage being a (β1-4) or an (β1-3) linkage, the tetrasaccharide being Neu5Ac(α2-3)GalGlcNAc(β1-3)Glc; or
   a type of O-glycosidic linkage between Gal and GlcNAc-OH in a disaccharide portion of the glycan sample, the type of the glycosidic linkage being a (β1-3), (β1-4) or a (β1-6) linkage.

In implementations, wherein the O-glycosidic linkage between Neu5Ac and Gal is an (α2-6) or an (α2-3) linkage, Neu5Ac(α2-6)Gal(β1-4)GlcNAc appears at a ratio of 0.758, and Neu5Ac(α2-3)Gal(β1-4)GlcNAc appears at a ratio of 0.531.

In implementations, wherein the O-glycosidic linkage between Gal and GlcNAc is a 031-4) or an (β1-3) linkage in the tetrasaccharide Neu5Ac(α2-3)GalGlcNAc(β1-3)Glc, Neu5Ac(α2-3)Gal(β1-4)GlcNAc(β1-3)Glc appears at a ratio of 0.403, and Neu5Ac(α2-3)Gal(β1-3)GlcNAc(β1-3)Glc appears at a ratio of 0.234.

In implementations, wherein the O-glycosidic linkage between Gal and GlcNAc-OH is a (β1-3), (β1-4) or a (β1-6) linkage, in the disaccharide Gal-GlcNAc-OH (in this disaccharide, GlaNAc is understood to be the reducing end), Gal(β1-3)GlcNAc-OH appears at a ratio of 0.685, Gal(β1-4)GlcNAc-OH appears at a ratio of 0.867, and Gal(β1-6)GlcNAc-OH appears at a ratio of 0.536.

In implementations, the ratios of 0.867, 0.758, 0.685, 0.536, 0.531, 0.403, or 0.234 for (m/z 204+m/z 366)/(m/z 204+m/z 366+m/z 274+m/z 292+m/z 454+m/z 472+m/z 495) all have variabilities associated with them. In implementations, the variability associated with the ratio of 0.758 is ±0.044. In implementations, the variability associated with the ratio of 0.531 is ±0.052. In implementations, the variability associated with the ratio of 0.403 is ±0.046. In implementations, the variability associated with the ratio of 0.234 is ±0.009. In implementations, the variability associated with the ratio of 0.685 is ±0.007. In implementations, the variability associated with the ratio of 0.867 is ±0.004. In implementations, the variability associated with the ratio of 0.536 is ±0.02.

In implementations, in the presently disclosed method of analyzing the structure of a glycan sample, the one or more first peaks are defined at an m/z of 204 and 495, and the one or more second peaks are defined at an m/z of 366 in an MSⁿ spectrum derived from fragmentation of a portion of the carbohydrate sample corresponding to an m/z of 657 in an MSⁿ⁻¹ spectrum,
wherein the predetermined ratio is 0.315, or 0.704, and
wherein the predetermined structural characteristic includes
   the presence of Neu5Ac(α2-3)Gal(β1-3)GalNAc-Ser or Gal(β1-3)[Neu5Ac(α2-6)]GalNAc-Ser oligosaccharide in the glycan sample.

Thus Neu5Ac(α2-3)Gal(β1-3)GalNAc-Ser appears at a ratio of 0.315, and Gal(β1-3)[Neu5Ac(α2-6)]GalNAc-Ser appears at a ratio of 0.704.

In implementations, the ratios of 0.315, and 0.704 for (m/z 204+m/z 495)/(m/z 204+m/z 495+m/z 366) all have variabilities associated with them. In implementations, the variability associated with the ratio of 0.685 is ±0.007. In implementations, the variability associated with the ratio of 0.315 is ±0.022. In implementations, the variability associated with the ratio of 0.704 is ±0.031.

In implementations, in the presently disclosed method of analyzing the structure of a glycan sample, the MS instrument includes an ion trap MS instrument. In implementations, the ion trap MS instrument employs collision-induced dissociation.

### EXPERIMENTAL EXAMPLES

### A) Linkage Memory in Underivatized Protonated Carbohydrates

### Methods

Reagents. The standards Gal(β1-4)GlcNAc, Gal(β1-6)-GlcNAc, Gal(β1-3)GlcNAc, Fuc(α1-4)GlcNAc, Fuc(α1-3)-GlcNAc, and Fuc(α1-6)GlcNAc were purchased from Carbosynth (Compton, U.K.). Deuterated ammonia (ND3, 99%) and 18O (98%) water were purchased from Cambridge Isotope Laboratories (Tewksbury, MA). All standards from stock solutions were verified by 1H NMR to ensure purity and conformational identity (FIG. 21) as described previously (Alonge, K. M.; Logsdon, A. F.; Murphree, T. A.; Banks, W. A.; Keene, C. D.; Edgar, J. S.; Whittington, D.; Schwartz, M. W.; Guttman, M. Quantitative analysis of chondroitin sulfate disaccharides from human and rodent fixed brain tissue by electrospray ionization-tandem mass spectrometry. Glycobiology 2019, 29, 847-860).

Ion Trap Mass Spectrometry. Carbohydrates were resuspended in LC-MS grade optima water and diluted to a working concentration of 10 μM in 0.1% formic acid. 180 labeling of the reducing end hydroxyl of GlcNAc was performed as described previously (Viseux, N.; de Hoffmann, E.; Domon, B. Structural Assignment of Permethylated Oligosaccharide Subunits Using Sequential Tandem Mass Spectrometry. Anal. Chem. 1998, 70, 4951-4959). Samples were analyzed on a LTQ Orbitrap mass spectrometer (Thermo Fisher Scientific) by direct infusion at a rate of ˜5 μL/min. The protonated precursor ion was subjected to CID using a range of normalized collision energies (CE) of 15-20. Full (MS1) scans were acquired followed by selection of the most intense fragment ions for further fragmentation with up to four rounds of MS/MS (MS4). All the multistage tandem MS experiments were performed in triplicate. Spectra were extracted and analyzed using Xcalibur software. MS analysis was coupled to liquid chromatography (Waters Acquity I-class) to resolve α/β anomers. A volume of 1 μL of a 10-50 μM solution of each was injected in pure water and resolved over a 2.1×150 mm 300 Å BEH amide column (Waters) using a gradient of 90% B to 55% B over 20 min (A, water with 0.1% formic acid; B, acetonitrile with 0.1% formic acid).

Ion Mobility-Mass Spectrometry (IM-MS). Ion mobility measurements were performed on a Waters Synapt G2-Si mass spectrometer. 50 μM glycans in 0.1% formic acid were directly infused at 5 μL/min. The sample cone voltage was optimized to generate each carbohydrate fragment ion of interest (m/z 366 for Gal-GlcNAcs or 350 for Fuc-GlcNAcs) and mass selected using the quadrupole to avoid any artifacts of ion decay during or after the IM stage. IM measurements in different gases were collected using the following settings: N₂ flow rate of 90 mL/min, traveling wave (TVV) velocity of 500 m/s, and height of 25 V; He flow rate of 120 mL/min, TW velocity, and height were 800 m/s and 8 V; and CO₂ flow rate of 42 mL/min, TW velocity, and height were 400 m/s and 16 V. The ion current was kept below 10×105 counts/s to avoid detector saturation, which may lead to broader than expected arrival time distributions (ATDs). For isomer ATDs that indicated two well-resolved species, the relative intensity of each species was approximated by fitting to two Gaussian distributions using custom Excel (Microsoft, Redmond, WA) scripts.

Gas-Phase Hydrogen/Deuterium Exchange (gHDX). Instrumental modifications to the Waters Synapt HDMS mass spectrometer to enable infusion of ND3 and He have been described previously.³² Analytes were resuspended in Optima LC-MS grade water from Fischer Scientific (Hampton, NH) and diluted to a working concentration of 400 μM in aqueous 0.1% formic acid.

An elevated cone voltage was used to generate water loss fragment ions in the source (m/z 350 and 366) which were then mass selected with the quadrupole and fragmented in the trap ion guide. A series of acquisitions were collected using a trap collision energy of 5, 10, and 17 for Gal(β1-4)GlcNAc and Gal(β1-3)GlcNAc, 5, 10, and 22 for Gal(β1-6)GlcNAc, and 5, 10, and 15 for Fuc(α1-4)GlcNAc, Fuc(α1-3)GlcNAc, and Fuc(α1-6)GlcNAc. gHDX was performed in the transfer TW ion guide (after the ion mobility stage and immediately before the TOF region).

Transfer wave heights of 8 V and multiple traveling wave velocities (8-512 m/s−1) were used to sample a temporal-range of ˜0.1-10 ms. Unless specified, all other MS settings were source temperature 100 ° C., capillary voltage 2.5 kV, sampling cone voltage 30 V, extraction cone voltage 3.3 V, trap CE 15 V, trap bias 8 V, IM TW velocity 100 m/s, IM TW height 5 V, RF confining voltage 350 V, transfer TW height 8 V. All samples were collected in triplicate, and all carbohydrates in this study were run on the same day to minimize any intersample variation.

As an additional control to ensure identical sampling of all analytes, the “lock” channel was used to infuse TrisMix, an exchange standard, cosampled along with the analyte of interest and used to verify that the exchange conditions were identical.³⁸ Data was batch converted using scripts in UniDec.³⁹ Quantification of deuterium uptake, binomial fitting, and bimodal deconvolution were performed using HX-Express v2.⁴⁰ Unlike solution HDX which generate a binomial isotopic distribution, the labile protons probed by gHDX can exchange at drastically different time-scales and cannot be modeled by a single binomial distribution.⁴¹ Instead a combination of two binomial distributions to account for relatively fast and slow exchanging sites in the exchange profile was applied and convoluted.

### Results

Characterization of Linkage and Anomeric Memory by Tandem MS. Two sets of isomeric disaccharides by MSⁿ to examine their fragmentation behavior were first investigated. Both sets of disaccharides, Gal(β1-x)GlcNAc and Fuc(α1-x)GlcNAc, where x=3, 4, and 6, are compositionally identical but different in their linkage configurations (FIG. 12A). Upon CID, the protonated precursors (m/z 384 for Gal-GlcNAcs and m/z 368 for Fuc-GlcNAcs) dissociate easily to lose water from their reducing end to form m/z 366 in Gal-GlcNAcs and m/z 350 in the case of Fuc-GlcNAcs (FIGS. 8A, B). Loss at the reducing end is confirmed by identical dissociation of an ¹⁸O-labeled fucosylated disaccharide (m/z 370), which dissociates primarily to m/z 350, thus confirming that the initial loss of water (−18 Da) is predominantly occurring from the reducing end of the disaccharides (FIGS. 12B, C).

In the case of the protonated Gal(β1-x)GlcNAcs, all three isomeric precursors fragment to the same product ions but with differing propensities. While m/z 222 is the classic Y1 ion formed by cleavage of the glycosidic bond, resulting in protonated GlcNAc, m/z 204 is formed by further loss of the reducing end of the protonated GlcNAc. The relative abundances of m/z 222 and 204 are visibly distinct for Gal(β1-6)GlcNAc compared to the other two disaccharides (FIG. 8A). Similar to the Gal(β1-x)GlcNAc disaccharides, MS² of the protonated Fuc(α1-x)-GlcNAc disaccharides (m/z 368) yield m/z 350, 222, and 204 (FIG. 8B). The MS² spectra of the fucosylated disaccharides is unique only for the (1-4) linkage. Further fragmentation of the water-loss fragment (MS³) of all six disaccharides revealed the same product ions, namely, m/z 204, 186, 168, 144, and 138 with varying relative abundances (FIG. 8C). Though MS³ (368→350→) of the fucosylated disaccharides yield the same fragment ions as Gal(β1-x)GlcNAcs, there are no unique patterns in the fragmentation spectra among the three linkage isomers (FIG. 8D).

Taking it a step further, MS4 (FIGS. 8E, F) was performed on all six disaccharides to fragment the GlcNAc (m/z 204) remnant, probing for signature CID fragmentation patterns. In addition to m/z 186, 168, 144 and 138, the MS4 (384→366→204→) spectra of all disaccharides yield m/z 126 and 84 which are primary and secondary products of m/z 204 (FIGS. 8E, F).

Interestingly, fragmentation of the 204 ion appears similar from Gal(β1-3)GlcNAc and Gal(β1-4)GlcNAc in their relative abundances and markedly distinct from Gal(β1-6)GlcNAc. Even more interesting is the MS⁴ of the fucosylated disaccharides (368→350→204→) that showed distinct fragmentation spectra for all three isomers (FIG. 8F). The observed differences were quantified by calculating the ratio of the relative abundances of the m/z 126 and 186 fragments from triplicate measurements (FIGS. 8E, F) and Table 1.

TABLE 1
m/z 126/186 ratios from direct infusion analysis of FucGlcNAc and GalGlcNAc isomers
and anomer resolved (LC-MSⁿ) measurements of FucGlcNAc. Anomers are indicated as either
early eluting [1] or late eluting [2].

| Direct-infusion | | | | | | |
|---|---|---|---|---|---|---|
| MSⁿ | 1-4 | | 1-6 | | 1-3 | |
| Gal-GlcNAc | 0.46 +/− 0.06 | | 1.48 +/− 0.25 | | 0.51 +/− 0.11 | |
| Fuc-GlcNAc | 0.47 +/− 0.06 | | 1.62 +/− 0.26 | | 1.04 +/− 0.33 | |
| LC-MSⁿ | 1^{st} eluting | 2^{nd} eluting | 1^{st} eluting | 2^{nd} eluting | 1^{st} eluting | 2^{nd} eluting |
| Fuc-GlcNAc | 0.91 +/− 0.03 | 0.34 +/− 0.01 | 1.69 +/− 0.03 | 1.67 +/− 0.04 | 1.26 +/− 0.06 | 0.75 +/− 0.03 |

TABLE 2
Matrix of p-values for differentiation of all combinations of isomers and anomers of
FucGlcNAc based on the m/z 126/186 ion ratio from T-tests. All species produce statistically
different 126/186 ion ratios, except for the two anomers of the (1-6) linked isomers (indicated in
italics).

| | (α1-3) [1] | (α1-3) [2] | (α1-4) [1] | (α1-4) [2] | (α1-6) [1] | (α1-6) [2] |
|---|---|---|---|---|---|---|
| (α1-3) [1] | X | 1.80E−04 | 7.40E−04 | 1.20E−05 | 4.10E−04 | 6.80E−04 |
| (α1-3) [2] | 1.80E−04 | X | 2.40E−03 | 3.10E−05 | 3.80E−06 | 8.30E−06 |
| (α1-4) [1] | 7.40E−04 | 2.40E−03 | X | 5.19E−06 | 6.50E−06 | 1.60E−05 |
| (α1-4) [2] | 1.20E−05 | 3.10E−05 | 5.19E−06 | X | 3.20E−07 | 1.00E−06 |
| (α1-6) [1] | 4.10E−04 | 3.80E−06 | 6.50E−06 | 3.20E−07 | X | 0.65 |
| (α1-6) [2] | 6.80E−04 | 8.30E−06 | 1.60E−05 | 1.00E−06 | 0.65 | X |

For both sets of disaccharides, the 126/186 ratio is significantly different for the (1-6) linkage as compared to the (1-4) linkage. For the fucosylated disaccharides, there was also a significant difference even between the (1-3) and (1-4) linkages. These varying ratios indicate that the fragmentation propensity and thereby the structures of the 204 ions differ when generated from precursors of different linkages.

All experiments thus far were performed using direct infusion and thus would probe both the α/β anomers that are present in solution. To examine how the reducing end anomeric configuration influences the structures generated from the precursor, the analyses using online hydrophilic interaction chromatography (HILIC) were repeated to resolve the α/β anomers of each disaccharide (FIG. 13). The MS4 spectra (384→366→204→) of all six disaccharides showed similar trends to the infusion data (FIGS. 8G, H), but in several cases there were large differences between the two anomers. Definitive assignment of the α and β anomers for the disaccharides cannot be made from this LC-MS data, and therefore each species was designated as early vs late eluting. The (1-6) linked Fuc-GlcNAc and Gal-GlcNAc show little dependence on the anomeric state of the precursor, but the (1-4) and (1-3) linked disaccharides generate more m/z 126 from the early eluting peak. This is most notable for Fuc(α1-4)GlcNAc and Gal(β1-3)GlcNAc where the early eluting peaks have more than double the relative intensity for the 126 peak.

Differences in the 126/186 ratios were found to be statistically significant between the 1-3 and 1-4 anomer pairs as assessed from triplicate measurements (p-values shown in FIG. 8H insets). For this comparison, also included was an analysis of the GlcNAc monosaccharide, for which the MSn relative product ion abundances (222→204→) were completely distinct from those generated from the disaccharides and did not produce statistically different ion ratios between the two anomers. It was noted that the signal strength of the eluting peaks in the LC-MSⁿ data showed less error than the direct infusion, reflected by the lower variation when comparing the triplicate measurements (Table 1). In addition to effective differentiation of anomers, the ions ratios for the LC-resolved disaccharide anomers also show a more pronounced distinction between of the three linkage isomers. The 1-3 and 1-4 linked Fuc-GlcNAc isomers have statistically unique m/z 126/186 ratios for all anomers (Table 2), which are also all distinct from the two 1-6 linkage anomers.

Characterization of Linkage and Anomeric Memory by IM-MS. Ion mobility spectrometry was applied to further characterize and compare the fragment ions from the isomeric disaccharides. The arrival time distributions (ATDs) of the m/z 366 and 350 ions from Gal-GlcNAc and Fuc-GlcNAc are shown in FIGS. 9A, B. Not surprisingly, each linkage isomer has a distinct ATD, consistent with a different structure. However, the (1-6) linked disaccharide isomers show two peaks, indicating the presence of two distinct structures. The relative intensity of each peak was dependent on the collision energy used to generate the fragments (FIG. 15). There is also a pronounced shoulder present for the Fuc(α1-3)GlcNAc, indicating a potential contribution from a second conformer (FIG. 9B, highlighted in yellow). Isolation of the 366/350 peaks with subsequent activation was used to produce and compare the ATDs of the m/z 204 ion produced from each disaccharide. While the ATDs for the m/z 204 fragments from both the (1-4) and (1-3)-linked precursors appear indistinguishable, the ATD from the (1-6) linked precursor is markedly distinct and for the Fuc(α1-6)GlcNAc it is visibly broader (FIGS. 9C, D).

To investigate the gas-phase structures of the fragment ions in greater detail, LC-IM-MS experiments were applied to resolve the α/β anomers and performed the analysis using N₂, He, and CO₂ as drift gases to maximize our ability to resolve subtle structural differences.⁴² Both anomers of the Gal-GlcNAc (m/z 366) and Fuc-GlcNAcs (m/z 350) yielded the same trends between the linkage isomers in all three drift gases, as the direct infusion data (FIGS. 9E, G). The ATDs from the (1-6) linked disaccharides, particularly in N₂ and CO₂, show clear evidence of multiple conformations that is consistent with multiple Gaussian distributions (FIG. 16). In several instances, the α/β anomers gave rise to distinct ATDs. This was most evident for Fuc(α1-3)GlcNAc where the two anomers were distinct in all three drift gases (FIG. 9G). The Gal(β1-3)GlcNAc also showed a slight difference between the anomers, most visible in N₂ and CO₂ (FIG. 9E). The ATDs for the (1-4) linked disaccharides were largely invariant among the anomers, with the possible exception of Gal(β1-4)GlcNAc in He, where the first eluting LC peak has a slightly later peak (FIG. 9E). The (1-6) linked ATDs did not show any obvious difference between the positions of the two peaks (FIG. 9E), but there was a subtle effect on the relative magnitude of the two peaks (FIG. 9G).

Taking it a step further, the mobilities of the m/z 204 ions formed from the m/z 366 and 350 ions from the LC-resolved peaks were examined. While the ATD from the (1-3) and (1-4) look indistinguishable, there is a clear shift in the ATDs from the (1-6) linked disaccharides (FIGS. 9F, H), just as seen from the infusion IM-MS data. While most of the ATDs were indistinguishable between the α/β anomers, the m/z 204 ions produced from the (1-3) linked disaccharides show some differences. This is most evident in Gal(β1-3)GlcNAc in N₂ and for Fuc(α1-3)GlcNAc in CO₂ and indicates that the anomeric configuration of a precursor can influence the structures of its fragment ions even after two stages of fragmentation. The m/z 138 ion, which forms one specific structure regardless of the precursor, 22 was invariant in all the samples, confirming that even the subtle differences noted above are not attributed to variations in IM-MS conditions (FIG. 17). Lastly, the m/z 204 ATDs were compared to that produced from monomeric GlcNAc (FIGS. 9F, H, bottom panel). While the ATDs for m/z 204 from the (1-4) and (1-3) linked disaccharides appear similar to the free GlcNAc in N₂ and He, they are visibly different from free GlcNAc in CO2.

Characterization of Linkage Memory by gHDX. gHDX was used as an orthogonal tool for resolving structural variations in the m/z 366, 350, and 204 fragments. The deuterium exchange kinetics for the 350 and 366 ions from each of the disaccharides is different, consistent with having different structures (FIGS. 10A, B). Close inspection of the exchange spectra reveals that the Gal(β1-6)GlcNAc linked 366 ion exists as an ensemble of two dominant structures while the (1-3) and (1-4) linked structures show a predominant isotopic distribution along with a small population of a highly deuterated species (FIG. 18A). For the Fuc-GlcNAc m/z 350 peaks, all of the linkages showed clear evidence of at least two populations, particularly at the longest exchange time points (FIG. 19A).

The m/z 204 fragments generated from all the disaccharides show a maximum exchange of 4 deuterium, consistent with the number of exchangeable protons but exhibit some notable differences in their gHDX rates (FIGS. 10C, D). For the Gal-GlcNAc series, the kinetics for the m/z 204 ion from the (1-3) and (1-4) linked precursors are within experimental error, while it is unique from Gal(β1-6)GlcNAc (FIG. 10C). Interestingly, the gHDX spectra of each m/z 204 shows evidence of a second, highly deuterated population (FIG. 18B, 2.5 ms time point), also suggesting the presence of multiple structures. In the case of the Fuc-GlcNAcs, the gHDX profile for all the m/z 204 were distinct (FIG. 10D).

The influence of the collision energy (CE) used to generate the fragment ions were also examined as this has been observed to influence gHDX kinetics (Uppal, S. S.; Beasley, S. E.; Scian, M.; Guttman, M. Gas-Phase Hydrogen/Deuterium Exchange for Distinguishing Isomeric Carbo-hydrate Ions. Anal. Chem. 2017, 89, 4737-4742). The profiles of the 366 and 350 ions are indeed offset by altered CE, especially with the 1-6 linkage structures (FIGS. 20A, B). In the case of the m/z 204 ions, higher CE results in a drop in the extent of exchange that predominantly affects the shorter time points. The exchange profile at the longer time points still distinguishes the (1-4) linked m/z 204 among Fuc-GlcNAcs and (1-6) linked m/z 204 among Gal-GlcNAcs. To test whether the altered CE was directly affecting the kinetics of deuterium exchange or simply offsetting the distribution of different structures generated, the isotopic distributions in the spectra that contained well-resolved populations (FIGS. 10E, F) were deconvoluted. Raising the CE used to generate the m/z 366, from 5 to 10, did not change the exchange kinetics of each individual population; rather it decreased the relative abundance of the highly deuterated population from 34 to 20%. While the gHDX data for m/z 204 did show evidence of multiple populations, these spectra could not be deconvoluted because of their insufficient separation (FIGS. 18B and 19B).

### Discussion

Multiple Conformations of Protonated Disaccharides. Computational and structural studies have provided a wealth of information on carbohydrate fragmentation (Bythell, B. J. et al., 2017; Struwe, W. B. et al., 2016; Rabus, J. M.; Abutokaikah, M. T.; Ross, R. T.; Bythell, B. J. Sodium-cationized carbohydrate gas-phase fragmentation chemistry: influence of glycosidic linkage position. Phys. Chem. Chem. Phys. 2017, 19, 25643-25652; Mucha, E.; Stuckmann, A.; Marianski, M.; Struwe, W. B.; Meijer, G.; Pagel, K. In-depth structural analysis of glycans in the gas phase. Chemical Science 2019, 10, 1272-1284; Rossich Molina, E.; Eizaguirre, A.; Haldys, V.; Urban, D.; Doisneau, G.; Bourdreux, Y.; Beau, J.-M.; Salpin, J.-Y.; Spezia, R. Characterization of Protonated Model Disaccharides from Tandem Mass Spectrometry and Chemical Dynamics Simulations. ChemPhysChem 2017, 18, 2812-2823; Yamagaki, T.; Fukui, K.; Tachibana, K. Analysis of Glycosyl Bond Cleavage and Related Isotope Effects in Collision-Induced Dissociation Quadrupole-Time-of-Flight Mass Spectrometry of Isomeric Trehaloses. Anal. Chem. 2006, 78, 1015-1022; Fentabil, M. A.; Daneshfar, R.; Kitova, E. N.; Klassen, J. S. Blackbody Infrared Radiative Dissociation of Protonated Oligosaccharides. J. Am. Soc. Mass Spectrom. 2011, 22, 2171-2178). A set of disaccharides structures that are common motifs within many glycoconjugates have been examined here. An emphasis was placed on the behavior of the water loss (B-type) fragments as these are generated in high abundance during CID of protonated glycoconjugates and are often used as diagnostic ions for identifying glycopeptides (Nwosu, C. C.; Seipert, R. R.; Strum, J. S.; Hua, S. S.; An, H. J.; Zivkovic, A. M.; German, B. J.; Lebrilla, C. B. Simultaneous and Extensive Site-specific N- and O-Glycosylation Analysis in Protein Mixtures. J. Proteome Res. 2011, 10, 2612-2624). The 180 labeling experiments (FIG. 12) confirm that the formation of B ions, m/z 366 (from Gal-GlcNAc) and 350 (from Fuc-GlcNAc) is by water loss occurring predominantly from the reducing end, consistent with several previous studies (Viseux, N. et al., 1998; Hofmeister, G. E.; Zhou, Z.; Leary, J. A. Linkage position determination in lithium-cationized disaccharides: tandem mass spectrometry and semiempirical calculations. J. Am. Chem. Soc. 1991, 113, 5964-5970). In both sets of disaccharides, these isomers were readily distinguishable by both IM-MS and gHDX, further showcasing each technique's ability to resolve carbohydrate isomers (FIGS. 9A, B, E, G and 10A, B).

The 350 and 366 ions can sample multiple conformations. This is most evident from the IM-MS and gHDX data of the (1-6) linked structures and to some extent in the (1-3) linked structures. The distribution of the two conformers of the (1-6) linked 366/350 ions is dependent on the collision energy used to generate the fragments (FIGS. 10E, F and FIG. 15). Furthermore, gHDX shows that even the (1-4) and (1-3) linked Fuc-GlcNAc isomers adopt multiple conformers as evidenced by multiple isotopic distributions (FIG. 19A). Therefore, it is likely that all of the 350 and 366 ions are inclusive of multiple structures, and IM-MS is simply unable to resolve some of them. These examples illustrate the highly orthogonal nature of gHDX and IM-MS. While gHDX differentiates ions based on their differences in gas-phase basicity and hydrogen bonding networks (Reyzer, M. L.; Brodbelt, J. S. Gas-phase H/D exchange reactions of polyamine complexes: (M+H)+, (M+alkali metal+), and (M+2H)2+. J. Am. Soc. Mass Spectrom. 2000, 11, 711-721; Wyttenbach, T.; Bowers, M. T. Gas phase conformations of biological molecules: The hydrogen/deuterium exchange mechanism. J. Am. Soc. Mass Spectrom. 1999, 10, 9-14), separation by IM-MS is based on the shape of the structure and its interactions with the buffer gas (Clowers, B. H. et al., 2005; Dwivedi, P. et al., 2007).

The large difference in the subpopulations seen by IM-MS for the (1-6) structures could be attributed to the extra rotational degree of freedom (O6-C6-C5), which bestows additional flexibility allowing a wider range of extended conformations, whereas the (1-3) and (1-4) are more constrained and can only access fewer conformations (details below). The discovery of “anomeric memory” has revealed a new paradigm where carbohydrate fragment structures are heavily influenced by the stereochemistry at leaving groups. Despite the loss of the reducing end, the original anomeric configuration influences the structure of B-type ions (Gray, C. J. et al., 2017; Ujma, J. et al., 2019).

This anomeric memory effect is directly observed in the water-loss B ions in the anomer-resolved LC-MS data. Interestingly, the stereochemistry at the anomeric end influenced nearly all of 350/366 ions as seen by IM-MS. In the case of the (1-4) and (1-3) linkages, the effect is a shift of the seemingly single ATD, whereas with the (1-6) linkage, it influences the relative intensity of each partially resolved peak (FIGS. 9E, G). It is likely that the distribution of the (1-3) and (1-4) structures is also similarly offset by the anomeric configuration, but the ATDs of the resulting structures are poorly resolved and manifest as an apparent change in the single peak position. Overall, the anomer-resolved data further exemplifies how anomeric memory occurs in a wide range of protonated carbohydrates.

Linkage Memory in Carbohydrate Fragments. The most intriguing finding from this study was obtained by examining the product ion resulting from a second round of fragmentation of the disaccharides. The m/z 350 and 366 ions fragment primarily to an abundant Y-type ion corresponding to the GlcNAc that has already lost its reducing end hydroxyl (m/z 204). By the methods employed, the structure(s) of the 204 ion generated from each linkage isomer was distinct, revealing that carbohydrate ions not only retain anomeric memory but also a linkage memory. Specifically, the 204 ions generated from the (1-6) disaccharides showed a longer ATD, indicating that they have an extended conformation compared to the other two 204 ions (FIGS. 9C, D, F, H).

The uniqueness of the 204 ion of the (1-6) linked disaccharides is also highlighted in subtle, but significant, differences in the gHDX profiles of the 204 ion (FIGS. 10C, D), along with reproducible differences in their fragmentation, particularly with respect to the relative abundance of its fragments, m/z 126 and 186 (FIGS. 8E-H). Lastly, it is noted that both MSn and IM-MS show that the behavior of the 204 generated from free GlcNAc monosaccharide is distinct from any of the equivalent fragments produced from the disaccharides (FIGS. 8G, H and 2F, H), further corroborating that prior linkage strongly influences the resulting structure of the 204 ion.

In some cases, the properties of the 204 ion revealed both linkage and anomeric memory. Particularly with the (1-4) linked 350 and (1-3) linked 366 disaccharides, the fragmentation propensity of the 204 is distinct for the α/β anomers, despite two rounds of fragmentation since the initial loss of the reducing end. At this stage, the GlcNAc has lost both its reducing end, which confers the α/β anomericity, and the sugar at the nonreducing end, which defined its linkage connectivity. Nevertheless, the properties of the 204 ion are characteristic of both its former anomericity and linkage connectivity, and thus it is encoded with memory from both ends.

In fact, not only does the attachment at a particular linkage site impact the resulting Y-type fragment ions, the linkage stereochemistry and identity of the attached sugar moiety also appear to matter. The CID products of the 204 ion from Gal(β1-3)GlcNAc and Fuc(α1-3)GlcNAc (FIGS. 8E, F) are distinct, implying that the identity of the nonreducing end monosaccharide (β-galactose versus α-fucose) is key along with the linkage type (where the GlcNAc is linked to another monosaccharide).

Further support is provided by the gHDX comparisons of the differently linked 204 ions of Gal-GlcNAc and Fuc-GlcNAcs, which show subtle differences for all six m/z 204 ions (FIG. 20E). However, it is likely that some of the differences observed in gHDX profiles are a result of the varying dissociation thresholds of each disaccharide, which may generate a slightly different distribution of fragment ion structures, as seen for the 1-6 linked disaccharides (FIGS. 10E, F).

Structural Rationalization of Linkage Memory. The mechanisms underlying linkage memory tie back to the structure(s) of the m/z 204 ion. Gray et al. proposed the presence of various bridged, open ring, and enol structures in the ensemble where the relative abundances of these structures is influenced by the original anomeric configuration (Gray, C. J. et al., 2017). Indeed, the present studies together with previous structural studies of protonated GlcNAc (Bythell, B. J. et al., 2017; Mookherjee, A. et al., 2018) can be used to rationalize the linkage memory. With disaccharides, the initial loss of water to generate m/z 350 or 366 may also lead to the formation of bridged structures (FIG. 11).

In the case of the (1-4) linkage, there is no hydroxyl at the O4 position and thus no O4-C1 bridged structure at the GlcNAc can form. Similarly, the (1-6) linked structure cannot form the O6-C1 bridged structure. Previous mapping of the fragmentation pathways of GlcNAc revealed that the 204 ion dissociates through two competing pathways to generate either (1) primarily m/z 186 through water loss or (2) m/z 126 through a ring rearrangement reaction (Mookherjee, A. et al., 2018). The O4-C1 bridge structure was proposed to initiate the ring rearrangement to form m/z 126. From current data, the (1-4) linked disaccharides generated the lowest amount of 126 (FIGS. 8E, F), which would be consistent with their inability to sample the O4-C1 bridged structure within their structural ensemble. In contrast, the (1-6) linked disaccharide generated the most 126 ion, which may generate relatively more O4-C1 since the formation of the O6-C1 pathway is not accessible. The (1-6) linked disaccharides not being able to generate a O6-C1 structure is also consistent with the IM-MS data. The 204 ion from the (1-6) linkage, which will have free rotation at the C5-C6-O6, appears less compact, while the (1-3) and (1-4) species, which can form the O6-C1 structure to keep the O6 more constrained, appear more compact with shorter ATDs.

It should be emphasized that the reaction mechanisms of the formation of the 350/366 ions and its tentative structures proposed here are likely oversimplified, yet they provide a plausible explanation for the observed linkage memory in the subsequent 204 ions.

The direct observation of multiple gas-phase structures from several disaccharides further strengthens the existing argument that carbohydrate fragment ions exist as ensembles of structures (Bythell, B. J. et al., 2017; Gray, C. J. et al., 2017; Rossich Molina, E. et al., 2017; Fentabil, M. A. et al., 2011). The structural ensemble of both B- and Y-type fragments are influenced by the identity of their precursors and both the reducing and nonreducing ends. Even an apparently simple chemically homogeneous structure can form several conformers that are poorly, partially, or well-resolved in the gas-phase. Furthermore, the presence of multiple conformers of both parent and fragment ions will have to be considered when benchmarking properties of carbohydrate ions, such as IM databases seeking to standardize collision cross section values.

Though protonated carbohydrates have proven more complex than metal adducted or derivatized carbohydrates, a fundamental understanding of carbohydrate fragments and the principles that govern their formation will be useful not only for structural studies but for the development of much needed technologies to characterize the complex biological functions of the diverse glycan and glycoconjugate repertoire. Overall, the diagnostic CID patterns, ion mobilities, and gHDX behavior of the differently linked Y fragments illustrate the potential of fragment ions in determining intact precursor carbohydrate structures.

### B) Multistage LC-MSⁿ for Automated Glycan Isomer Assignment of Glycopeptides

A database of fragmentation ratios for structural elucidation. Data sets on a broad range of glycan standards representing biologically relevant sialylated glycans found on both O-linked and N-linked glycans have been exhaustively collected. All data were collected using collision induced dissociation (CID) within an ion trap at a wide range of collision energies (CE) to ensure the ratios we report are CE-independent. From this extensive dataset the most reliable ion ratios that are diagnostic of glycan structures have now been computed (Table 3).

TABLE 3
Fragment ion ratios for a variety of oligosaccharides at either the MS² or MS³ level. The
specific ion ratios used are indicated in the columns on the right. The rows highlighted in bold
represent a case where both MS² and MS³ level ion ratios of the 366 ion were acquired, and show
consistent results.

| | | | | (186 + 204)/(186 + | | (204 + 366)/(204 + | | | |
|---|---|---|---|---|---|---|---|---|---|
| | | | | 204 + 256 + 274 + | | 274 + 292 + 366 + | | (204 + 495)/(204 + | |
| 657 Product Ratios | | | | 292 + 454 + 495) | | 454 + 472 + 495) | | 366 + 495) | |
| MS1 | MS2 | MS3 | structure | avg | stdev | avg | stdev | avg | stdev |
| 675 | 657 | | NeuAc5(a2-6)Gal(b1-4)GlcNAc-OH | 0.426 | 0.030 | 0.758 | 0.044 | 0.252 | 0.024 |
| 675 | 657 | | NeuAc5(a2-3)Gal(b1-4)GlcNAc-OH | 0.219 | 0.007 | 0.531 | 0.052 | 0.258 | 0.039 |
| 873 | 657 | | NeuAc5(a2-3)Gal(b1-4)GlcNAc(b13)Glc | 0.127 | 0.008 | 0.403 | 0.046 | 0.212 | 0.025 |
| 873 | 657 | | NeuAc5(a2-3)Gal(b1-3)GlcNAc(b13)Glc | 0.129 | 0.006 | 0.234 | 0.009 | 0.476 | 0.015 |
| 762 | 657 | | NeuAc5(a2-3)Gal(b1-3)GalNAc-Ser | 0.122 | 0.008 | 0.325 | 0.005 | 0.315 | 0.022 |
| 762 | 657 | | Gal(b1-3)[NeuAc5(a2-6)]GalNAc-Ser | 0.081 | 0.010 | 0.255 | 0.004 | 0.704 | 0.031 |
| | | | | | | (13 + 168)/ | | | |
| | | | | | | (126 + 138 + 144 + | | (145 + 163)/ | |
| 366 Product Ratios | | | | 138/(138 + 144) | | 168 + 186) | | (145 + 163 + 204) | |
| MS1 | MS2 | MS3 | structure | avg | stdev | avg | stdev | avg | stdev |
| 675 | 657 | 366 | NeuAc5(a2-6)Gal(b1-4)GlcNAc-OH | 0.971 | 0.006 | 0.843 | 0.007 | 0 | |
| 675 | 657 | 366 | NeuAc5(a2-3)Gal(b1-4)GlcNAc-OH | 0.970 | 0.005 | 0.811 | 0.011 | 0 | |
| 873 | 657 | 366 | NeuAc5(a2-3)Gal(b1-4)GlcNAc(b1-3)Glc | 0.979 | 0.005 | 0.885 | 0.007 | 0 | |
| 873 | 657 | 366 | NeuAc5(a2-3)Gal(b1-3)GlcNAc(b1-3)Glc | 0.924 | 0.003 | 0.621 | 0.012 | 0 | |
| 762 | 366 | | NeuAc5(a2-3)Gal(b1-3)GalNAc-Ser | 0.570 | 0.012 | 0.135 | 0.013 | 0.0069 | 0.0011 |
| 762 | 657 | 366 | NeuAc5(a2-3)Gal(b1-3)GalNAc-Ser | 0.544 | 0.017 | 0.108 | 0.013 | 0.0044 | 0.0057 |
| 762 | 657 | 366 | Gal(b1-3)[NeuAc5(a2-6)]GalNAc-Ser | 0.560 | 0.014 | 0.082 | 0.008 | 0.0072 | 0.0003 |
| 762 | 366 | | Gal(b1-3)[NeuAc5(a2-6)]GalNAc-Ser | 0.556 | 0.013 | 0.098 | 0.010 | 0.0014 | 0.0005 |
| 384 | 366 | | Gal(b1-3)GalNAc-OH | 0.598 | 0.009 | 0.168 | 0.014 | 0.0330 | 0.0080 |
| 384 | 366 | | Gal(b1-6)GalNAc-OH | 0.622 | 0.010 | 0.164 | 0.012 | 0.0620 | 0.0020 |
| 384 | 366 | | Gal(b1-3)GlcNAc-OH | 0.934 | 0.004 | 0.685 | 0.007 | 0 | |
| 384 | 366 | | Gal(b1-4)GlcNAc-OH | 0.977 | 0.003 | 0.867 | 0.004 | 0 | |
| 384 | 366 | | Gal(b1-6)GlcNAc-OH | 0.918 | 0.002 | 0.536 | 0.020 | 0 | |

Data from both MS² and MS³ spectra were collected to ensure ratios are reliable at different stages of MS, which can be observed using some larger sialylated oligosaccharides where MS² selection and fragmentation of the 366 ion produces similar results to MS³ spectrum where the 657 ion is first selected, fragmented and the 366 ion is further selected and fragmented (bold entries in Table 3). By using a combination of the fragment ion ratios it is possible to distinguish (2-6) vs. (2-3) linked sialylation, as well as 1-3, 1-4, and 1-6 linkage between galactose and N-acetylglucosamine (GlcNAc). Lastly, a metric has now been identified using the 145 and 163 ions generated during fragmentation of N-acetylgalactosamine (GalNAc) containing disaccharides that can differentiate between 1-6 and 1-3 linkage, which has thus far not been possible. The errors in the table reflect replicates among different collision energies, thus ensuring these ratios to be useful for structural determination with a wide range of acquisition settings.

Acquisition protocol for glycopeptide LC-MSⁿ. With the aim of making MS³ and MS⁴ feasible on an LC timescale with complex samples, data-dependent acquisition protocols have now been established within Xcalibur (Thermo) to identify, select, and subsequently target glycopeptides for MS³ and MS⁴ analysis. Glycopeptide obtained from a combined Operator and trypsin digest of bovine fetuin were resolved over C18 nanoLC chromatography.

These proteases were found optimal for generating several glycopeptides containing a single O-linked glycan. Both MS³ and MS⁴ spectra could be obtained from data-dependent analysis (FIG. 22). However, due to the limited time window during peak elution, there is often insufficient time for collecting multiple spectra to estimate uncertainty for each calculated ion ratio. To this end, the relationship between ratio error and the total ion counts within MS³ and MS⁴ spectra have been mapped using infusions of standards at a range of dilutions.

The plots show the expected relationship: the more counts of the ions the lower the error associated with each calculated ratio (FIG. 5). Using this data set it is now possible to approximate a ratio error from a single measurement using the total ion counts within the spectra. This step is useful for correct interpretation of glycan structural interpretation as in many cases the total intensity falls short of what is used for confident structural assignment. Knowing this, it has been possible to go back and re-optimize the LC-MSⁿ acquisition program to selectively target ions where that are likely to generate the ion counts for precise ratio measurements (e.g. >2000 counts for the 138/(138+144), to reliably distinguish GlcNAc vs. GalNAc).

### C) Establishment of Various Aims

In various implementations, a multi-stage tandem MS approach is established that integrates characteristic fragment ion ratios and analytical software tools to provide detailed glycan structural analysis. This approach utilizes existing MS instrumentation that is already widely available within glycobiology and proteomics communities, and therefore can be seamlessly adopted by researchers.

In one implementation, a database of fragment ion abundance ratios at the MS³ and MS⁴ level for discrimination of isomeric protonated glycans using collision induced dissociation (CID) is established. A library of commercial synthetic oligosaccharides, purified milk oligosaccharides, and glycopeptides are used to catalogue the fragmentation patterns of various structures that encompass biologically relevant carbohydrate structures. This study is conducted on two ion trap MS platforms under different conditions to test the reproducibility and robustness of fragment abundance ratio metrices across different platforms.

In one implementation, a LC-MS acquisition protocol for obtaining maximum glycan structural information in a short time window is developed. Glycopeptides from well-studied glycoproteins are analyzed by LC-MS protocols that trigger subsequent multistage tandem MS (MSⁿ) upon detection of specific glycan fragment ions. A combination of MSⁿ scans are optimized to assess the feasibility of performing the MSⁿ steps on a chromatographic timescale and the cleanest (highest signal to noise) and most informative ions to use for structural interpretation are identified.

In one implementation, Byonic and Byomap software, which Protein Metrics' tools for detached glycan and glycopeptide analysis, respectively, are modified to output glycan/glycopeptide fragment peaks, intensities, and matched glycan compositions. Prototype software are developed to score glycan structures that best explain the observed MSⁿ data.

FIG. 24 shows that the resulting approach utilizes glycopeptide level tandem MS data to determine the peptide sequence, the site of glycan attachment, the glycan composition, and specific linkage and anomeric configurations across the glycan structure. Successful implementation of this tool overcomes existing limitations with MS-based characterization of glycosylation and propels future advances in glycoproteomics and characterization of biotherapeutics.

As shown in FIG. 25, for glycopeptides, tandem MS can identify the peptide sequence, the attachment site of glycans, the overall sugar composition, and minimal glycan linkage information (Schumacher, K. N. & Dodds, E. D. A case for protein-level and site-level specificity in glycoproteomic studies of disease. Glycoconj J 33, 377-385, doi:10.1007/s10719-016-9663-5 (2016); Riley, N. M., Malaker, S. A., Driessen, M. D. & Bertozzi, C. R. Optimal Dissociation Methods Differ for N- and O-Glycopeptides. Journal of Proteome Research 19, 3286-3301, doi:10.1021/acs.jproteome.0c00218 (2020)).

However, MS often cannot differentiate among potential structural isomers, so glycan structural details remain ambiguous and are only inferred from likely biological pathways. Alternative methods for complete detailed structural elucidation of glycans require their prior release from the glycoprotein: a step which sacrifices linkage information. Previously, there was no single approach that could provide both detailed structural analysis of glycans and their specific attachment site to the protein (‘site-specific’ glycan analysis). A MS-based approach compatible with existing proteomics platforms that could distinguish subtle glycan isomers has the potential to reduce the ambiguity in assigning glycan structures, ushering a new era in glycoproteomics. With this in mind, fragment ion abundances of underivatized protonated glycans can be explored to distinguish glycan isomers. While MSⁿ for structural elucidation of carbohydrates has been well-established for some time, it has largely been limited to examining permethylated or metal adducted oligosaccharides (Reinhold, V., Zhang, H., Hanneman, A. & Ashline, D. Toward a Platform for Comprehensive Glycan Sequencing. Molecular & Cellular Proteomics: MCP 12, 866-873, doi:10.1074/mcp.R112.026823 (2013)), which is not amenable to site-specific glycan analysis as glycoproteomics is performed on protonated underivatized glycopeptides.

Protein Metrics and Bios (FIG. 26) was used for MS/MS-based proteomics search and LC-MS peptide mapping. This platform offers about 25 predefined workflows, as well as easy definition of new workflows. Each workflow calls one or more of the underlying programs. Detached glycan analysis was handled by the same program as peptide mapping, Byomap. Glycan assignments are typically at the level of composition, but supports user-specified LC retention time information for isomer assignment. Extracted ion chromatogram (XIC) quantitation and MS² annotation were performed.

Glycopeptide analysis was performed in Byonic, which handles LC-MS/MS data with many fragmentation modes including CID, high energy collision dissociation (HCD), electron transfer dissociation (ETD), and ETD with HCD (EThcD). Carbohydrate fragments were annotated and provided some structural information. FIG. 27 shows an example EThcD spectrum of a glycopeptide from a patient-derived IgM antibody that identifies a bisecting GlcNAc in the glycan structure. Previously, there was no known commercial proteomics tool (SEQUEST, Mascot, etc.) that offered such significant glycoproteomics capabilities.

Previously, Byomap and Byonic had two at least two limitations: 1) glycan composition is determined automatically, but exact structural assignment is left to the user; 2) tandem mass spectra are analyzed individually and independently. These limitations can be addressed through implementations of the present disclosure.

Several recent reports have highlighted the use of ion abundance ratios for protonated carbohydrate ions to discern between classes of isomers (Halim, A. et al. Assignment of Saccharide Identities through Analysis of Oxonium Ion Fragmentation Profiles in LC-MS/MS of Glycopeptides. Journal of Proteome Research 13, 6024-6032, doi:10.1021/pr500898r (2014); Mookherjee, A., Uppal, S. S. & Guttman, M. Dissection of Fragmentation Pathways in Protonated N-Acetylhexosamines. Anal Chem 90, 11883-11891, doi:10.1021/acs.analchem.8b01963 (2018); Pett, C. et al. Effective Assignment of alpha2,3/alpha2,6-Sialic Acid Isomers by LC-MS/MS-Based Glycoproteomics. Angewandte Chemie (International ed. in English) 57, 9320-9324, doi:10.1002/anie.201803540 (2018)). However, the previous approaches are limited with respect to information content and reproducibility across different MS platforms.

In one implementation, CID performed in an ion trap mass analyzer can be used for reproducible measurement of fragmentation propensities. Due to resonance excitation, the primary fragment ions generated are not activated further so subsequent cleavage pathways are limited. This enables more reproducible fragmentation across different instruments. In contrast, in beam-type CID, as performed in a quadrupole (e.g., in Q-TOF), or in HCD, primary fragment ions are further activated and can undergo extensive secondary fragmentation. While this is beneficial because it generates more types of ions, it can hinder reproducible fragmentation that is essential for reliable structural assignments. FIG. 28 illustrates the benefits of ion trap CDID wherein MSⁿ is used for monosaccharide differentiation. N-acetyl glucosamine (GlcNAc) and N-acetylgalactosamine (GalNAc), two monosaccharides found in nearly all glycan structures, produce characteristic abundance ratios for the m/z 138 and 144 fragments. The ratios are consistent across a wide range of collision energy (CE) settings, as secondary fragmentation is minimized. It has also been found that the ratio of the m/z 84 and 126 ions relative to the sum of all ions from 204 contains useful information about prior linkage configuration (FIG. 28C) (Mookherjee, A. et al., Anal Chem 90, 11883-11891 (2018)). Furthermore, the ion ratios are similar when analyzed on two different ion trap instruments (Thermo Orbitrap Fusion, and a Thermo LTQ; compare circles and diamonds in FIG. 28B, C). In contrast, the same fragmentation using HCD or performed in in a Q-TOF (Waters Synapt G2-Si) shows a higher dependence on CE, due to secondary fragmentations (FIG. 28B, C triangles and squares). By using ion trap CID, one can make use of instrumentation that is widely used among proteomics and glycomics communities. Therefore such technology will be readily accessible to a large group of researchers.

In one implementation, a combination of MSⁿ spectra from a glycopeptide precursor is collected. Glycopeptides generate several carbohydrate oxonium-type fragment ions that are often used for recognizing glycopeptides (Pap, A., Klement, E., Hunyadi-Gulyas, E., Darula, Z. & Medzihradszky, K. F. Status Report on the High-Throughput Characterization of Complex Intact O-Glycopeptide Mixtures. J Am Sac Mass Spectrom 29, 1210-1220, doi:10.1007/s13361-018-1945-7 (2018)).

FIGS. 29A-F illustrate the level of information available from MS³ of the commonly observed m/z 657 oxonium ion that represents a trisaccharide composed of a neuraminic acid (Neu5Ac), a hexose, presumably galactose (Gal), and a N-acetylhexosamine (GlcNAc or GalNAc). From examination of three standard trisaccharides, the information from MS³ can effectively differentiate between two potential linkage configurations of Neu5Ac (α2-3 vs. α2-6). The difference in abundance of the 204 vs. the 274 and 292 can be readily observed from the spectra of the three disaccharides (FIG. 29A). The observed differences tie back to different propensity to fragment either at the Neu5Ac bond to generate 292 and 272 (B₁, and B₁—H₂O ions), or at the second glycosidic bond to generate 204 and 454. The resulting ratios remain distinct across a wide range of CEs (FIG. 29B, compare purple vs. red and teal).

To further interrogate the trisaccharide structure, the m/z 366 is isolated and fragmented to produce another key set of ions: m/z 126, 138, 168, and 186 (FIG. 29C). The fragmentation of three GalGlcNAc linkage isomers (β1-3, β1-4, β1-6), were examined, and a very unique and robust ion abundance ratio capable of differentiating among all three, and with little dependence on CE was observed (FIG. 29D). Similar ratios were obtained when m/z 366 was isolated directly from the precursor (MS3), or from m/z 657 (MS4), illustrating the robustness of this approach. The combination of information from the spectra of the 657 and 366 can now define the linkage configuration present in the trisaccharide. A similar approach has successfully resolved oligosaccharides containing various linkages of fucose (Fuc) by MSⁿ analysis using additional glycan ions (e.g. m/z 350, 512) (Abhigya, M., Sanjit S., U., Taylor A., M. & Miklos, G. Linkage Memory in Underivatized Protonated Carbohydrates (2020)).

Furthermore, the m/z 204 from either 366 or 657 was isolated for further fragmentation (MS⁴). This step provides additional ion ratios that can be used to assign the identity of the N-acetylhexosamine (GlcNAc vs. GalNAc as seen in FIG. 28A). It is noted that the 138:144 ratio can often be obtained directly from the 366 MS³ scan to differentiate among GlcNAc and GalNAc (FIG. 29C), however the measurements were less precise. Furthermore, the distribution of fragments from the 204 provide additional data to assign linkage. It has recently been reported that Y-ions from oligosaccharides retain memory of their prior linkage states. This ‘linkage memory effect’ is a consequence of the varied structure of the m/z 204 ion from different prior linkages, which in turn exhibit different dissociation pathways. The resultant fragment ion propensities can be used to differentiate among potential prior linkage configurations. From the comparison of the three Gal-GlcNAc disaccharides described above, the drastic difference in the (84+126):(all fragments of 204) ratio is observed, which again is independent of CE (FIG. 29F). Multiplexed data obtained from a series of MSⁿ scans therefore provide specific and sometimes redundant structural details of the glycan. A summary of the stages of fragmentation, the relevant ions, and the information obtained from each step is summarized in FIG. 30.

In implementations, three aims were achieved to evaluate the feasibility of developing a reliable MSⁿ tool incorporated with glycoproteomics software. Extensive MSⁿ data were collected on a variety of glycan samples. Optimal LC-MSⁿ acquisition protocols were developed for glycopeptides. In parallel, software was implemented to extract, curate, and query MS″ information for glycan structural prediction.

Aim 1: Building a database of fragmentation ratios for structural elucidation. MSⁿ was first collected on synthetic carbohydrate standards, purified milk oligosaccharides, and released glycans from glycoproteins. Subsequently, MSⁿ data was collected on glycopeptides from proteolytic digests of well-studied glycoproteins including: Bovine Fetuin, Erythropoietin, C1 inhibitor, hemopexin, GP1 b alpha, bovine submaxillary mucins, and porcine gastric mucins (Riley, N. M. et al., Journal of Proteome Research 19, 3286-3301, doi:10.1021/acs.jproteome.0c00218 (2020); Royle, L., Dwek, R. A. & Rudd, P. M. Determining the structure of oligosaccharides N- and O-linked to glycoproteins. Curr Protoc Protein Sci Chapter 12, Unit 12 16 (2006); Huang, L. J. et al, Identification of protein O-glycosylation site and corresponding glycans using liquid chromatography-tandem mass spectrometry via mapping accurate mass and retention time shift. Journal of chromatography. A 1371, 136-145, doi:10.1016/j.chroma.2014.10.046 (2014)). Standards were analyzed by LC-MSⁿ using hydrophilic interaction chromatography (HILIC). This step helped to minimize potential interference from sample impurities and also resolve the α/β anomers that exist on the reducing end, which can influence fragmentation propensities (Mookherjee, A. et al., Anal Chem 90, 11883-11891, doi:10.1021/acs.analchem.8b01963 (2018); Gray, C. J. et al. Bottom-Up Elucidation of Glycosidic Bond Stereochemistry. Anal Chem 89, 4540-4549, doi:10.1021/acs.analchem.6b04998 (2017)). This ensures that glycans were structurally well-defined and homogeneous so that fragmentation data are reliable.

Fragmentation data were obtained on two ion trap platforms: a Thermo L TQ ion trap and a Thermo Fusion Orbitrap. Access to the L TQ allowed the performance of much of the MSⁿ intensity ratio mapping and sample screening and optimization steps Access to a Thermo L TQ Orbitrap and nano LC systems are also available. While preliminary data suggested that the intensity ratios are similar across different ion trap platforms, having MSⁿ data sets from two different commonly used MS instruments added rigor to this study and ensured downstream reproducibility. Each sample was collected with multiple dilutions and across a range of CEs (20-60). Other parameters that may influence fragmentation propensities are also adjusted including He pressure, trap Q, and ion transfer optics to understand their impact and ensure all potential sources of variability are accounted for. Variability in fragment ratios due to source settings, ion optics, gas pressure, collision energy, and across ion trap platforms allow the establishment of ‘ratio thresholds or bounds’ and a “ratio range” for each specific carbohydrate structure in the MS³ database, that was used later to assess certainty when assigning structure.

One of the inherent caveats of working with protonated glycans is the well-established phenomenon of structural rearrangements during CID, particularly fucose migration. While new information regarding the mechanism of the migration process is emerging, the exact mechanism is yet be understood (Wuhrer, M., Koeleman, C. A., Hokke, C. H. & Deelder, A. M. Mass spectrometry of proton adducts of fucosylated N-glycans: fucose transfer between antennae gives rise to misleading fragments. Rapid Commun Mass Spectrom 20, 1747-1754 (2006); Mucha, E. et al. Fucose Migration in Intact Protonated Glycan Ions: A Universal Phenomenon in Mass Spectrometry. Angewandte Chemie (International ed. in English) 57, 7440-7443, doi:10.1002/anie.201801418 (2018)). Thus, fucose containing oligosaccharide ions (m/z 350, 512) were monitored specifically for potential migration events, and this also enabled better interpretation of the profiles of the original native structure.

Aim 1 was successfully performed by: 1) performing validation that fragment ion abundance ratios from oxonium ions (m/z 204, 350, 366, etc.) were unambiguously characteristic to each specific isomeric structure; and 2) determining that the fragment abundance ratios were reproducible across multiple ion trap instruments.

Aim 2. Optimize an acquisition protocol for glycopeptide LC-MSⁿ. As a first step for benchmarking LC-MSⁿ protocol, suitable LC conditions were set up for resolving glycopeptides. As conventional C18 chromatography is often unable to resolve isomeric glycans within glycopeptides (‘glycoforms’). HILIC and porous graphilic chromatography (PGC) were applied to resolve glycoforms (Melmer, M., Stangler, T., Premstaller, A. & Lindner, W. Comparison of hydrophilic-interaction, reversed-phase and porous graphitic carbon chromatography for glycan analysis. Journal of chromatography. A 1218, 118-123, doi:10.1016/j.chroma.2010.10.122 (2011); Huang, Y., Nie, Y., Boyes, B. & Orlando, R. Resolving Isomeric Glycopeptide Glycoforms with Hydrophilic Interaction Chromatography (HILIC). Journal of biomolecular techniques: JBT 27, 98-104, doi:10.7171/jbt.16-2703-003 (2016)). Initial experiments were to utilize data-dependent (DDA) acquisition methods to analyze eluting peptides for MS/MS, enabling the targeting of glycopeptides that contain characteristic diagnostic ions (Pap, A. et al., J Am Soc Mass Spectrom 29, 1210-1220, doi:10.1007/s13361-018-1945-7 (2018)). Subsequent DDA methods were to select glycopeptides and perform MS³ and MS⁴ on oxonium ions from the targeted glycopeptide precursors. When working on an LC timescale there was a limited acquisition time window during peak elution from the column (ranging from 15 seconds to over 1 minute). Though MS/MS acquisition is relatively rapid (lens to hundreds of milliseconds), the additional isolation and fragmentation steps for MS³ and MS⁴, along with the potential need for longer scans for improving signal to noise, limits data collection speed. It is noted that these disadvantages were platform dependent as the mass isolation and scanning speeds of the two instruments utilized here (L TQ and Fusion Orbitrap) are different, and limitations observed with the L TQ may not be present on the Fusion. Ultimately, the a set of acquisition protocols that produce the highest level of information on glycan structure in the shortest amount of time were generated and described herein.

Optimization of MSⁿ on the Fusion Orbitrap was to incorporate the use of additional MS² fragmentation including HCD, ETD, and EThCD for more detailed information on the peptide portion, including site-localization of glycans in cases where the attachment site is ambiguous (Riley, N. et al., Journal of Proteome Research 19, 3286-3301, doi:10.1021/acs.jproteome.0c00218 (2020)). It is noted that ETD and HCD methods and analysis software are well-established, so these are evaluated to test their synergy and limitations when combined with MSⁿ approach for glycan structural prediction. A consequence of working within the time constraints of collecting LC-MSⁿ data was that the standard approach for error assessment from replicates is not practical. An alternative approach for estimating an error from a single spectrum was to utilize the total signal intensity information; as signal quality correlates with precision. A dilution series of a handful of standards are performed that cover each oxonium ion of interest, each in triplicate, on both the LTQ and the Fusion to map the relationship between signal intensity (total ion counts) and precision (error from replicates). Knowing this relationship enabled each intensity ratio measured on a given MS platform to have a corresponding level of uncertainty, Therefore, if the spectra contained weak signal, this error assessment prevented erroneous interpretation or misassignment of glycan structural information.

One anticipated caveat with glycopeptide analysis is that even with various orthogonal modes of chromatography, several isomeric glycoforms may perfectly co-elute, and therefore be co-sampled. Co-isolation and fragmentation of isomeric structures can generate anomalous fragmentation propensities that could be at best uninterpretable, and at worst misleading. To account for this potential pitfall, a series of analyses were performed on two or more carbohydrate isomers mixed at different ratios to assess the overall effect on the MSⁿ ion ratios at each stage. This helped to identify the source of the varying MSⁿ ratios and potentially highlight any key spectral features or specific ion ratios that can reveal the presence of co-sampled isomers. This is also useful for proper analysis of complex N-linked glycans where the multiple branches may have different isomeric structures that fragment to produce the same m/z oxonium ions.

Success of Aim 2 was the validation that MS³ and MS⁴ can be performed on biologically relevant samples within a time frame of LC-MS and still provide the required level of information for structure prediction. Specifically, it was determined whether the majority of detectable glycopeptides from a mixture of glycoproteins will generate the signal at the MS³ and MS⁴ stages for reliable structural assessment.

Aim 3. Software for Glycan Structure Assignments. Software was developed using diagnostic peaks and MSⁿ fragmentation propensities to infer glycan structure for both detached glycans and glycopeptides.

Byomap and Byonic can output key peak information (m/z's, intensities, and annotations) and composition-level glycan or glycopeptide assignments for each spectrum. The new software reads in this information, along with a database of glycan structures, and output all the structures in the database tied for the highest score. For Phase I, peak information and glycan compositions were passed in a plain text format (.csv), and software was written in Python. For Phase II, intermediate data is kept in SQLite and software was written in C++.

Key Peaks. Most of the key peaks (126, 138, 144, 204, 366, etc.) were oxonium ions with fixed m/z values. For both detached glycans and glycopeptides, however, there are also peaks with “movable” masses, that is, the glycan fragments with a reducing end label (including +18.011 Da for aldehyde or +20.026 Da for alditol) or a peptide attached. The total number of key peaks is not large, <100 for mammalian glycans, so key peaks can be represented as either peak lists (m/z and intensity pairs) or as fixed-length vectors (e.g., 4th entry is always m/z 204 intensity) with zeros for missing entries. In either case, lists or vectors were linked into a tree structure to represent the “parental” relationships in the MS″ analysis. (Links between mass spectra have many other benefits beyond MSⁿ analysis: connecting ETD/HCD pairs, detecting in-source dissociation, combining scans to improve signal-to-noise ratio or search speed.) It may be assumed that that each spectrum included a “nearly correct” glycan composition, because glycan mass translates almost directly to composition. There are two notable exceptions: 2 deoxyhexoses (dHex2) has mass only 1.01 Da greater than Neu5Ac, and dHex1Neu5Gc1 has exactly the same mass as Hex1Neu5Ac1. Both Byomap and Byonic can confuse these alternatives, so the structure assignment code described in this implementation also corrects compositional ambiguities.

Glycan Database. Phase I used small glycan databases suitable for the target glycans and glycoproteins to be studied. Each glycan structure had complete linkage information, and uncertainty was represented simply by the list of alternate structures. This approach gives a straightforward way to utilize biological knowledge (unlikely glycans are left out) as well as a way to measure success (reduction in the number of alternatives).

The GlyTouCan database (https://lglytoucan.org) contained an accession for each level of uncertainty, and GNOme (see https:/lwww.glygen.org) provided a “subsumption ontology” for GlyTouCan accessions. Coverage, however, was not necessarily complete. For example, the glycan in FIG. 4 in GlyTouCan could not be found, but the same structure with α3 and α6 arms swapped is accession G24888ZY. At least for Phase I, glycan structures were pre-specified and uncertainty was managed with sets rather than codes, in order to pose the most realistic structure elucidation problems. Sets of alternative structures previously with MALDI-TOF and TOF-TOF (Burlak, C. et al. N-linked glycan profiling of GGTA1/CMAH knockout pigs identifies new potential carbohydrate xenoantigens. Xenotransplantation 20, 277-291, doi:10.1111/xen.12047 (2013)) and ESI-MS1 and MS2 spectra (Goldberg, D. et al. Automated N-glycopeptide identification using a combination of single- and tandem-MS. J Proteome Res 6, 3995-4005, doi:10.1021/pr070239f (2007)) were utilized.

Scoring Glycan Structures. To mitigate risk, two different approaches were taken to glycan scoring: a “handcrafted” classifier, and a more automatic approach that learned classification features from less processed data. The handcrafted approach included two steps: (1) scoring topologies using diagnostic peaks, and (2) assigning monosaccharides and linkages using fragment abundance ratios. Here “topology” is used to mean a tree graph of monosaccharide nodes (e.g., HexNAc) connected by edges without linkage information. Previously (Goldberg, D., Bern, M., Li, B. & Lebrilla, C. B. Automatic determination of O-glycan structure from fragmentation spectra. J Proteome Res 5, 1429-1434, doi:10.1021/pr060035j (2006)), summed tandem MS spectra (i.e., no parental relationships) were used. Topologies were scored for detached O-glycans by (# ions observed)−ε·(# ions missing), where theoretical ions were predicted by successive loss of monosaccharides from the non-reducing termini, and ε was a small constant (e.g., 0.01) so that missing ions are used to break ties. Starting with this simple scorer, it was iteratively improved using parental relationships and peak intensities.

After step (1), the set of glycan structures were reduced to a small number of topologies; for example, the large peak at m/z 690 in FIG. 27 might eliminate all topologies without a connected subgraph containing 3 Hex and 1 HexNAc, which (depending upon the glycan database) might require a bisecting GlcNAc. It is to be noted that step (1) alone already gave some structural information from MS² spectra without further fragmentation.

Step (2) then fills in missing information in the topologies from biosynthetic rules and available MSⁿ spectra on both specific monosaccharides (e.g., GlcNAc vs. GalNAc, as shown in FIG. 28) and linkage information (e.g., α2-3 vs α2-6 linked Neu5Ac, as shown in FIG. 29).

Furthermore, a more modern, automated machine learning (ML) approach for glycan structure identification is described. This approach utilizes training data (MSⁿ data) paired with labeled outputs (glycan structures or substructures) for supervised learning. An MSⁿ data set was specified by fixed-length vectors of key peaks, e.g. an MS² spectrum of 657 followed by an MS³ spectrum of 366. ML was used to produce a method for classifying unknown inputs. Several ML methods were explored, including classification trees and CNNs (convolutional neural networks). Data-intensive ML methods such as CNNs can benefit from synthetic or semisynthetic data in which a generative model (learned “adversarially”) produces training data for the classification algorithm. This “Software 2.0” approach was compared to the handcrafted approach.

The software was tested in three stages: 1) on purified disaccharides and trisaccharides; 2) tests on mixtures of released glycans from standard reference proteins; and 3) a mixture of glycopeptides from glycoperoteins described in Aim 1. The goal was automatic structural classification at human expert level performance. Numerical goals depended upon the difficulty of the problem; for example, GlcNAc vs GalNAc was relatively easy and close to perfect classification is expected. A high accuracy rate (>90%) was also observed for assigning linkage of antennary galactose (β1-3 vs. β1-4) and terminal sialic acid (α.2-3 vs. α.2-6). Correct assignment of fucosylation linkage is expected to be a more complicated iterative process, dependent upon the emerging information obtained from Aims 1 and 2, in part due to the likelihood of migration during CID.

### Example Clauses

The following clauses represent various implementations of the present disclosure.
1. A method of analyzing the structure of a glycan sample, the method including: receiving data indicative of one or more spectra of mass-to-charge ratio (m/z) versus relative abundance of the glycan sample from a mass spectrometer (MS) instrument; generating a ratio according to the following Equation:

$$\frac{a}{a + b}$$

wherein a is a magnitude of one or more first peaks in the one or more spectra and b is the magnitude of one or more second peaks in the one or more spectra; determining that the ratio is within a range of a predetermined ratio; based on determining that the ratio is within the range of the predetermined ratio, determining that a predetermined structural characteristic is present in the glycan sample; and outputting an indication of the predetermined structural characteristic in the glycan sample.
2. The method of clause 1, wherein the glycan sample includes a protonated glycan including an oligosaccharide.
3. The method of clause 1 or 2, wherein the protonated glycan includes an O-glycan, an N-glycan, or a sialylated glycan.
4. The method of any one of clauses 1 to 3, wherein the glycan includes an oligosaccharide, and monosaccharides of the oligosaccharide are linked through O-glycosidic linkages.
5. The method of any one of clauses 1 to 4, wherein the predetermined structural characteristic includes: an identity of the one or more monosaccharides or oligosaccharides of the glycan; a connectivity of the O-glycosidic linkages of the one or more oligosaccharides of the glycan; or a configuration of anomeric carbons in the one or more monosaccharides or oligosaccharides of the glycan.
6. The method of any one of clauses 1 to 5, further including: determining the variability of the predetermined ratio based on a number of ion counts associated with the one or more spectra.
7. The method of any one of clauses 1 to 6, further including: obtaining, by the MS instrument, the one or more spectra by passing the glycan sample through the MS instrument, and generating, by one or more analog-to-digital converters (ADCs), the data indicative of one or more spectra.
8. The method of clause 7, wherein obtaining, by MS instrument, the one or more spectra includes: obtaining, by the MS instrument, a first MS spectrum (MS¹ spectrum) by passing the glycan sample through the MS instrument a first time; obtaining a portion of the carbohydrate sample corresponding to one or more peaks of the MS¹ spectrum; and obtaining, by the MS instrument, a second MS spectrum (MS² spectrum) by passing the portion of the carbohydrate sample through the MS instrument a second time.
9. The method of Implementation 8, wherein the portion of the glycan sample is a first portion of the glycan sample, and wherein, obtaining, by the MS instrument, the one or more spectra further includes: obtaining a second portion of the glycan sample corresponding to one or more peaks of the MS² spectrum; and obtaining, by the MS instrument, a third MS spectrum (MS³ spectrum) by passing the second portion of the glycan sample through the MS instrument a third time.
10. The method of Implementation 9, wherein obtaining, by the MS instrument, the one or more spectra further includes: obtaining a third portion of the carbohydrate sample corresponding to one or more peaks of the MS³ spectrum; and obtaining, by the MS instrument, a fourth MS spectrum (MS⁴ spectrum) by passing the third portion of the glycan sample through the MS instrument a fourth time.
11. The method of any one of clauses 7 to 10, wherein prior to obtaining, by the MS instrument, the one or more spectra by passing the glycan sample through the MS instrument, the glycan sample is passed through a liquid chromatography (LC) instrument to generate a liquid chromatogram, and wherein obtaining, by the MS instrument, the one or more spectra by passing the glycan sample through the MS instrument the first time comprises passing, through the MS instrument, a portion of the glycan sample corresponding to a particular peak in the liquid chromatogram.
12. The method of any one of clauses 1 to 11, wherein the one or more first peaks are defined at an m/z of 138 and the one or more second peaks are defined at an m/z of 144 in an MSⁿ spectrum derived from fragmentation of a portion of the sample corresponding to m/z 204 in an MSⁿ⁻¹ spectrum, wherein the predetermined ratio is 0.37, 0.81, or 0.73 and wherein the predetermined structural characteristic includes: the presence of an isomer in the glycan sample, the isomer including N-acetylgalactosamine (GalNAc), N-acetylglucosamine (GlcNAc), or N-acetylmannosamine (ManNAc); or the presence at the reducing end of an oligosaccharide in the glycan sample, of GalNAc or GlcNAc.
13. The method of any one of clauses 1 to 12, wherein the one or more first peaks are defined at an m/z of 84 and 126 and the one or more second peaks are defined at an m/z of 138, 144, 168, and 186 in an MSⁿ spectrum derived from fragmentation of a portion of the sample corresponding to m/z 204 in an MSⁿ⁻¹ spectrum, wherein the predetermined ratio is 0.21, 0.20, or 0.42 and wherein the predetermined structural characteristic includes: a type of a O-glycosidic linkage between two monosaccharides in a disaccharide portion of the glycan sample, the type of the O-glycosidic linkage being 1-3, 1-4, or 1-6 linkage.
14. The method of clause 13, wherein the two monosaccharides include: Fucα and GlcNAc; or Galβ and GlcNAc.
15. The method of any one of clauses 1 to 14, wherein the one or more first peaks are defined at an m/z of 138 and 168, and the one more second peaks are defined at an m/z of 126, 144, and 186 in an MSⁿ spectrum derived from fragmentation of a portion of the sample corresponding to an m/z of 366 or an m/z of 350 in an MSₙ₋₁ spectrum, wherein the predetermined ratio is wherein the predetermined ratio is 0.621, 0.843, or 0.536 and wherein the predetermined structural characteristic includes: a type of a O-glycosidic linkage between two monosaccharides in an oligosaccharide portion of the glycan sample, the type of the O-glycosidic linkage being 1-3, 1-4, or 1-6 linkage.
16. The method of clause 15, wherein the two monosaccharides include: Galβ and GlcNAc; or Fucβ and GlcNAc.
17. The method of any one of clauses 1 to 16, wherein: the one or more first peaks are defined at an m/z of 186 and 204, and the one or more second peaks are defined at an m/z of 256, 274, 292, 454, and 495 in an MSⁿ spectrum derived from fragmentation of a portion of the carbohydrate sample corresponding to an m/z of 657 in an MSⁿ⁻¹ spectrum; wherein the predetermined ratio is 0.426 or 0.219, and wherein the predetermined structural characteristic includes: a type of O-glycosidic linkage between two monosaccharides of a disaccharide portion of the glycan sample, the type of the glycosidic bond linkage being an (α2-6) or an (α2-3) linkage.
18. The method of clause 17, wherein: one of the two monosaccharides is Neu5Ac; or one of the two monosaccharides in Neu5Ac, and the other is Gal.
19. The method of any one of clauses 1 to 18, wherein the one or more first peaks are defined at an m/z of 204 and 366, and the one or more second peaks are defined at an m/z of 274, 292, 454, 472 and 495 in an MSⁿ spectrum derived from fragmentation of a portion of the carbohydrate sample corresponding to an m/z of 657 in an MSⁿ⁻¹ spectrum, wherein the predetermined ratio is 0.867, 0.758, 0.685, 0.536, 0.531, 0.403, or 0.234, and wherein the predetermined structural characteristic includes: a type of O-glycosidic linkage between Neu5Ac and Gal in a disaccharide portion of the glycan sample, the type of the glycosidic linkage being an (α2-6) or an (α2-3) linkage; a type of O-glycosidic linkage between Gal and GlcNAc in a tetrasaccharide portion of the glycan sample, the type of the glycosidic linkage being a (β1-4) or an (β1-3) linkage, the tetrasaccharide being Neu5Ac(α2-3)GalGlcNAc(β1-3)Glc; or a type of O-glycosidic linkage between Gal and GlcNAc-OH in a disaccharide portion of the glycan sample, the type of the glycosidic linkage being a (β1-3), (β1-4) or a (β1-6) linkage.
20. The method of clause 1, wherein the MS instrument includes an ion trap MS instrument.
21. The method of clause 20, wherein the MS instrument employs collision-induced dissociation (CID).
22. A system including: at least one processor; and receiving data indicative of one or more spectra of mass-to-charge ratio (m/z) versus relative abundance of a glycan sample from a mass spectrometer (MS) instrument; generating a ratio according to the following Equation:

$$\frac{a}{a + b}$$

wherein a is a magnitude of one or more first peaks in the one or more spectra and b is the magnitude of one or more second peaks in the one or more spectra; determining that the ratio is within a range of a predetermined ratio; based on determining that the ratio is within the range of the predetermined ratio, determining that a predetermined structural characteristic is present in the glycan sample; and outputting an indication of the predetermined structural characteristic in the glycan sample.
23. A system including: a mass spectrometer (MS) instrument configured to generate data indicative of one or more spectra of mass-to-charge ratio (m/z) versus relative abundance of a glycan sample; an output device configured to output a report indicating a predetermined structural characteristic present in the glycan sample; and a processor configured to: generate a ratio according to the following Equation:

$$\frac{a}{a + b}$$

wherein a is a magnitude of one or more first peaks in the one or more spectra and b is the magnitude of one or more second peaks in the one or more spectra; determine that the ratio is within a range of a predetermined ratio; based on determining that the ratio is within the range of the predetermined ratio, determine that the predetermined structural characteristic is present in the glycan sample.

As will be understood by one of ordinary skill in the art, each implementation disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or implementations of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations of this invention are described herein, including the best mode known to the inventors for carrying out the invention. Of course, variations on these described implementations will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the implementations of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred implementations of the present invention only and are presented in the cause of providing what is believed to be the most useful and readily understood description of the principles and conceptual aspects of various implementations of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of analyzing the structure of a glycan sample, the method comprising:
receiving data indicative of one or more spectra of mass-to-charge ratio (m/z) versus relative abundance of the glycan sample from a mass spectrometer (MS) instrument;
generating a ratio according to the following Equation:
$$\frac{a}{a + b}$$
wherein a is a magnitude of one or more first peaks in the one or more spectra and b is the magnitude of one or more second peaks in the one or more spectra;
determining that the ratio is within a range of a predetermined ratio;
based on determining that the ratio is within the range of the predetermined ratio, determining that a predetermined structural characteristic is present in the glycan sample; and
outputting an indication of the predetermined structural characteristic in the glycan sample.

2. The method of claim 1, wherein the glycan sample comprises a protonated glycan comprising an oligosaccharide.

3. The method of claim 2, wherein the protonated glycan comprises an O-glycan, an N-glycan, or a sialylated glycan.

4. The method of claim 1, wherein the glycan comprises an oligosaccharide, and wherein monosaccharides of the oligosaccharide are linked through O-glycosidic linkages.

5. The method of claim 1, wherein the predetermined structural characteristic comprises:
an identity of the one or more monosaccharides or oligosaccharides of the glycan;
a connectivity of the O-glycosidic linkages of the one or more oligosaccharides of the glycan; or
a configuration of anomeric carbons in the one or more monosaccharides or oligosaccharides of the glycan.

6. The method of claim 1, further comprising:
determining the range of the predetermined ratio based on a number of ion counts associated with the one or more spectra.

7. The method of claim 1, further comprising:
obtaining, by the MS instrument, the one or more spectra by passing the glycan sample through the MS instrument, and
generating, by one or more analog-to-digital converters (ADCs), the data indicative of one or more spectra.

8. The method of claim 7, wherein obtaining, by MS instrument, the one or more spectra comprises:
obtaining, by the MS instrument, a first MS spectrum (MS¹ spectrum) by passing the glycan sample through the MS instrument a first time;
obtaining a portion of the carbohydrate sample corresponding to one or more peaks of the MS¹ spectrum; and
obtaining, by the MS instrument, a second MS spectrum (MS² spectrum) by passing the portion of the carbohydrate sample through the MS instrument a second time.

9. The method of claim 8, wherein the portion of the glycan sample is a first portion of the glycan sample, and
wherein, obtaining, by the MS instrument, the one or more spectra further comprises:
obtaining a second portion of the glycan sample corresponding to one or more peaks of the MS² spectrum; and
obtaining, by the MS instrument, a third MS spectrum (MS³ spectrum) by passing the second portion of the glycan sample through the MS instrument a third time.

10. The method of claim 9, wherein obtaining, by the MS instrument, the one or more spectra further comprises:
obtaining a third portion of the carbohydrate sample corresponding to one or more peaks of the MS³ spectrum; and
obtaining, by the MS instrument, a fourth MS spectrum (MS⁴ spectrum) by passing the third portion of the glycan sample through the MS instrument a fourth time.

11. The method of claim 7, wherein prior to obtaining, by the MS instrument, the one or more spectra by passing the glycan sample through the MS instrument, the glycan sample is passed through a liquid chromatography (LC) instrument to generate a liquid chromatogram, and
wherein obtaining, by the MS instrument, the one or more spectra by passing the glycan sample through the MS instrument the first time comprises passing, through the MS instrument, a portion of the glycan sample corresponding to a particular peak in the liquid chromatogram.

12. The method of claim 1, wherein the one or more first peaks are defined at an m/z of 138 and the one or more second peaks are defined at an m/z of 144 in an MSⁿ spectrum derived from fragmentation of a portion of the sample corresponding to m/z 204 in an MSⁿ⁻¹ spectrum,
wherein the predetermined ratio is 0.37, 0.81, or 0.73 and
wherein the predetermined structural characteristic comprises:
the presence of an isomer in the glycan sample, the isomer comprising N-acetylgalactosamine (GalNAc), N-acetylglucosamine (GlcNAc), or N-acetylmannosamine (ManNAc); or
the presence at the reducing end of an oligosaccharide in the glycan sample, of GalNAc or GlcNAc.

13. The method of claim 1, wherein the one or more first peaks are defined at an m/z of 84 and 126 and the one or more second peaks are defined at an m/z of 138, 144, 168, and 186 in an MSⁿ spectrum derived from fragmentation of a portion of the sample corresponding to m/z 204 in an MSⁿ⁻¹ spectrum,
wherein the predetermined ratio is 0.21, 0.20, or 0.42 and
wherein the predetermined structural characteristic comprises:
a type of a O-glycosidic linkage between two monosaccharides in a disaccharide portion of the glycan sample, the type of the O-glycosidic linkage being 1-3, 1-4, or 1-6 linkage.

14. The method of claim 1, wherein the one or more first peaks are defined at an m/z of 138 and 168, and the one more second peaks are defined at an m/z of 126, 144, and 186 in an MSⁿ spectrum derived from fragmentation of a portion of the sample corresponding to an m/z of 366 or an m/z of 350 in an MSⁿ⁻¹ spectrum,
wherein the predetermined ratio is wherein the predetermined ratio is 0.621, 0.843, or 0.536 and
wherein the predetermined structural characteristic comprises:
a type of a O-glycosidic linkage between two monosaccharides in an oligosaccharide portion of the glycan sample, the type of the O-glycosidic linkage being 1-3, 1-4, or 1-6 linkage.

15. The method of claim 1, wherein:
the one or more first peaks are defined at an m/z of 186 and 204, and the one or more second peaks are defined at an m/z of 256, 274, 292, 454, and 495 in an MSⁿ spectrum derived from fragmentation of a portion of the carbohydrate sample corresponding to an m/z of 657 in an MSⁿ⁻¹ spectrum;
wherein the predetermined ratio is 0.426 or 0.219, and
wherein the predetermined structural characteristic comprises: a type of O-glycosidic linkage between two monosaccharides of a disaccharide portion of the glycan sample, the type of the glycosidic bond linkage being an (α2-6) or an (α2-3) linkage.

16. The method of claim 1, wherein the one or more first peaks are defined at an m/z of 204 and 366, and the one or more second peaks are defined at an m/z of 274, 292, 454, 472 and 495 in an MSⁿ spectrum derived from fragmentation of a portion of the carbohydrate sample corresponding to an m/z of 657 in an MSⁿ⁻¹ spectrum,
wherein the predetermined ratio is 0.867, 0.758, 0.685, 0.536, 0.531, 0.403, or 0.234, and
wherein the predetermined structural characteristic comprises:
a type of O-glycosidic linkage between Neu5Ac and Gal in a disaccharide portion of the glycan sample, the type of the glycosidic linkage being an (α2-6) or an (α2-3) linkage;
a type of O-glycosidic linkage between Gal and GlcNAc in a tetrasaccharide portion of the glycan sample, the type of the glycosidic linkage being a (β1-4) or an (β1-3) linkage, the tetrasaccharide being Neu5Ac(α2-3)GalGlcNAc(β1-3)Glc; or
a type of O-glycosidic linkage between Gal and GlcNAc-OH in a disaccharide portion of the glycan sample, the type of the glycosidic linkage being a (β1-3), (β1-4) or a (β1-6) linkage.

17. The method of claim 1, wherein the MS instrument comprises an ion trap MS instrument.

18. The method of claim 17, wherein the MS instrument employs collision-induced dissociation (CID).

19. A system comprising:
at least one processor; and
receiving data indicative of one or more spectra of mass-to-charge ratio (m/z) versus relative abundance of a glycan sample from a mass spectrometer (MS) instrument;
generating a ratio according to the following Equation:
$$\frac{a}{a + b}$$
wherein a is a magnitude of one or more first peaks in the one or more spectra and b is the magnitude of one or more second peaks in the one or more spectra;
determining that the ratio is within a range of a predetermined ratio;
based on determining that the ratio is within the range of the predetermined ratio, determining that a predetermined structural characteristic is present in the glycan sample; and
outputting an indication of the predetermined structural characteristic in the glycan sample.

20. A system comprising:
a mass spectrometer (MS) instrument configured to generate data indicative of one or more spectra of mass-to-charge ratio (m/z) versus relative abundance of a glycan sample;
an output device configured to output a report indicating a predetermined structural characteristic present in the glycan sample; and
a processor configured to:
generate a ratio according to the following Equation:
$$\frac{a}{a + b}$$
wherein a is a magnitude of one or more first peaks in the one or more spectra and b is the magnitude of one or more second peaks in the one or more spectra;
determine that the ratio is within a range of a predetermined ratio;
based on determining that the ratio is within the range of the predetermined ratio, determine that the predetermined structural characteristic is present in the glycan sample.
